(19) United States
(12) Patent Application Publication
(10) Pub. No.: US 2022/0243213 A1
(43) Pub. Date: Aug. 4, 2022

(54) ANTI-CRISPR INHIBITORS

(71) Applicants: THE REGENTS OF THE UNIVERSITY OF CALIFORNIA, OAKLAND, CA (US); THE GOVERNING COUNCIL OF THE UNIVERSITY OF TORONTO, TORONTO (CA)

(72) Inventors: Joseph Bondy-Denomy, Oakland, CA (US); Adair Borges, Oakland, CA (US); Jenny Yujie Zhang, Oakland, CA (US); Beatriz Osuna, SR., Oakland, CA (US); Sabrina Stanley, Toronto, Ontario (CA); Alan Davidson, Toronto, Ontario (CA)

(21) Appl. No.: 17/613,894

(22) PCT Filed: May 29, 2020
(86) PCT No.: PCT/US2020/035403

§ 371 Date: Nov. 23, 2021

Related U.S. Application Data
(60) Provisional application No. 62/854,085, filed on May 29, 2019.

(51) Int. Cl. C12N 15/78; C12N 1/20; C12N 7/00; C12N 9/22; C12N 15/11

(52) U.S. Cl. CPC C12N 15/78; C12N 1/20; C12N 7/00; C12N 9/22; C12N 2795/10042; C12N 2310/20; C12N 2800/80; C12N 2795/00022; C12N 15/11

(57) ABSTRACT

The present disclosure provides compositions and methods for introducing or enhancing Aca activity in prokaryotic cells. The provided compositions and methods can be used to inhibit Acr activity in prokaryotic cells, thereby enhancing endogenous or exogenous CRISPR-Cas activity. Cells, polynucleotides, plasmids, phage, and other elements for practicing the present methods are also provided.

### CROSS REFERENCE TO RELATED APPLICATIONS

The present application claims priority to U.S. Provisional Pat. Appl. No. 62/854,085, filed on May 29, 2019, which application is incorporated herein by reference in its entirety.

### STATEMENT REGARDING FEDERALLY SPONSORED RESEARCH OR DEVELOPMENT

This invention was made with government support under grants OD021344 and GM127489 awarded by the National Institutes of Health. The government has certain rights in the invention.

### BACKGROUND OF THE INVENTION

Bacteria possess a multitude of defense mechanisms to protect against the ubiquitous threat of bacteriophage (phage) infection. One such mechanism, the CRISPR-Cas system, “immunizes” bacteria and archaea against invading genetic elements like phages by incorporating short sequences of DNA from these invaders into their chromosome (Datsenko et al., 2012; Levy et al., 2015; Yosef et al., 2012). These sequences are subsequently transcribed and processed into small RNAs known as CRISPR RNAs (crRNAs) that bind to CRISPR-associated (Cas) proteins to form ribonucleoprotein interference complexes. These complexes survey the cell, recognize foreign nucleic acids through complementarity with their crRNAs, and ultimately destroy these foreign elements through the intrinsic nuclease activity of the Cas proteins (Barrangou et al., 2007; Brouns et al., 2008; Garneau et al., 2010; Marraffini and Sontheimer, 2008). CRISPR-Cas systems are diverse, comprising six distinct types, each with multiple subtypes (Makarova et al., 2015). In many bacteria studied to date, CRISPR-Cas systems are expressed in the absence of phage infection (Agari et al., 2010; Cady et al., 2011; Deltcheva et al., 2011; Juranek et al., 2012; Young et al., 2012), ensuring that they are primed to defend against a previously encountered phage at any given time. Upon phage infection, CRISPR-Cas may be upregulated to ensure that a sufficient number of interference complexes accumulate to successfully neutralize an invading phage (Young et al., 2012).

In response to CRISPR-Cas, phages and other mobile genetic elements endure by encoding protein inhibitors of CRISPR-Cas systems, known as anti-CRISPRs (Bondy-Denomy et al., 2013; Pawluk et al., 2016b). Anti-CRISPR proteins function, e.g., by preventing CRISPR-Cas systems from recognizing foreign nucleic acids or by inhibiting their nuclease activity (Bondy-Denomy et al., 2015; Chowdhury et al., 2017; Dong et al., 2017; Guo et al., 2017; Harrington et al., 2017; Pawluk et al., 2017; Wang et al., 2016). Anti-CRISPRs are encoded in diverse viruses and other mobile elements found in, for example, the Firmicutes, Proteobacteria, and Crenarchaeota phyla. They show a tremendous amount of sequence diversity, with 40 entirely distinct anti-CRISPR protein families now identified. Among these families are inhibitors of type I-C, I-D, I-E, I-F, II-A, II-C, and V-A systems, which function through a range of mechanisms.

Anti-CRISPR proteins display no common features with respect to sequence, predicted structure, or genomic location of the genes encoding them. However, a remarkable characteristic of anti-CRISPR genes is that they are almost invariably found upstream of a gene encoding a protein containing a helix-turn-helix (HTH) DNA-binding domain (FIG. 1). Seven different families of genes encoding these HTH-containing proteins have been designated as anti-CRISPR associated (aca). Members of aca gene families have been identified in divergent contexts including phages, prophages, and conjugative elements in diverse bacterial species (Bondy-Denomy et al., 2013; Marino et al., 2018; Pawluk et al., 2016a; Pawluk et al., 2016b). The ubiquity of aca genes adjacent to anti-CRISPR genes has provided a key bioinformatic tool for the identification of diverse anti-CRISPR families (Marino et al., 2018; Pawluk et al., 2016a; Pawluk et al., 2016b). The widespread occurrence of aca genes implies that they play an important role in anti-CRISPR systems, yet to date their function has remained unknown.

The ability of CRISPR-Cas systems to specifically target nucleic acids through their guide RNA sequences has opened the way to a vast number of applications. CRISPR-Cas is used, for example, as a way to eliminate pathogens with precision (e.g. Yosef et al., 2015; Pursey et al. 2018, Citorik et al. 2014; Bikard & Barrangou 2017), for gene editing, to regulate gene expression, or for nucleic acid labeling and imaging studies (see, e.g., Greene, 2018; Adli, Nat Commun. 2018 May 15; 9(1):1911; Pursey et al., 2018).

A potential problem with such CRISPR-mediated approaches, however, is that many prokaryotes contain resident prophages, plasmids, and conjugative islands that encode anti-CRISPR (Acr) proteins, which are capable of inhibiting both endogenous and exogenous CRISPR-Cas systems. In “self-targeting” bacterial strains, for example, in which a match exists between a spacer DNA sequence within the CRISPR locus and a prophage sequence within the bacterial genome, Acr proteins maintain the CRISPR-Cas system in an inactive state; in the absence of such inactivation, the Cas proteins would recognize and cleave the matching sequence within the prophage DNA, thereby killing the cell. Thus, if CRISPR activity were desired in such a cell for any purpose, e.g., to selectively kill the cell or for genome editing, the presence of the Acr would render the strategy ineffective.

There is thus a need for new methods and compositions for overcoming the inhibitory effects of anti-CRISPR proteins in situations where CRISPR activity is desired. The present disclosure satisfies this need and provides other advantages as well.

### BRIEF SUMMARY OF THE INVENTION

The discovery that “anti-CRISPR associated” (aca) genes transcriptionally repress anti-CRISPR (acr) loci has provided a tool to repress anti-CRISPR expression and thereby ensure the activation of CRISPR-Cas function in prokaryotic cells. acr loci have corresponding aca repressor genes whose products bind to the acr promoters and inhibit them. It is thus possible to use aca genes, e.g., by inducing their expression in prokaryotic cells, to repress the expression of their corresponding Acr proteins and thereby ensure the activity of CRISPR-Cas systems in the cell. Accordingly, one can deliver an Aca-encoding polynucleotide to a cell where CRISPR-Cas-mediated gene editing or bacterial killing is desired, but where an Acr inhibits, or potentially inhibits, endogenous or exogenous CRISPR-Cas function. The present methods and compositions can be used even when it is not known in advance whether or not the targeted prokaryotic cell contains an acr gene in its genome, or what type of acr gene it may contain. Simply by providing one or more Acas to the cell, e.g. alone or in conjunction with one or more guide RNAs and/or Cas proteins, existing or potentially existing Acrs in the cell can be inactivated, thereby allowing the activation of endogenous and/or exogenous Cas and the consequent targeting of nucleic acids as directed by one or more guide RNAs.

In one aspect, methods of activating CRISPR-Cas are provided to target a nucleic acid in a bacterial cell expressing an anti-CRISPR (Acr) protein, comprising introducing an anti-CRISPR associated (Aca) protein into the cell, wherein the Aca protein represses expression of the Acr protein and thereby allows the Cas protein to target the nucleic acid as directed by a guide RNA.

In some embodiments, the method further comprises introducing the guide RNA into the bacterial cell. In some embodiments, the Cas protein is endogenous to the bacterial cell. In some embodiments, the Cas protein is exogenous to the bacterial cell. In some embodiments, the method further comprises introducing the Cas protein into the bacterial cell. In some embodiments, the introducing step comprises introducing a polynucleotide encoding the Cas protein into the cell.

In some embodiments, the introducing step comprises introducing a polynucleotide encoding the Aca protein into the cell, wherein the Aca protein is expressed in the cell. In some embodiments, the introducing step comprises contacting a bacterial cell with a phage that encodes the Aca protein, wherein the phage introduces a polynucleotide encoding the Aca protein into the bacterial cell and the bacterial cell expresses the Aca protein. In some embodiments, the introducing step comprises contacting the bacterial cell with a conjugation partner bacterium comprising a polynucleotide that encodes the Aca protein, wherein the Aca protein or a polynucleotide encoding the Aca protein is introduced from the conjugation partner bacterium to the bacterial cell by bacterial conjugation.

In some embodiments, the method occurs within a mammalian host of the bacterial cell. In some embodiments, the bacterial cell resides in the gut of the mammalian host. In some embodiments, the mammalian host is a human. In some embodiments, the nucleic acid is DNA. In other embodiments, the nucleic acid is RNA. In some embodiments, the DNA is in the bacterial chromosome. In some embodiments, the nucleic acid is within a prophage, plasmid, or other mobile genetic element. In some embodiments, the Cas protein induces a double strand break in the nucleic acid. In some embodiments, the Cas protein binds to the nucleic acid and activates or represses transcription. In some embodiments, the Cas protein is labeled. In some embodiments, the Aca protein is substantially identical (e.g., at least 60%, 70%, 80%, 90%, 95% identical) to one of SEQ ID NOS: 1-27 or SEQ ID NOS: 50-60.

In another aspect, the present disclosure provides a polynucleotide comprising a promoter operably linked to a sequence encoding an Aca protein that is substantially identical (e.g., at least 60%, 70%, 80%, 90%, 95% identical) to one of SEQ ID NOS: 1-27 or SEQ ID NOS: 50-60, wherein the promoter is heterologous to the sequence. In some embodiments, the promoter is a constitutive promoter. In some embodiments, the promoter is an inducible promoter.

In another aspect, the present disclosure provides a phage or plasmid comprising a polynucleotide encoding an anti-CRISPR associated (Aca) protein, wherein the polynucleotide is heterologous to the phage or plasmid. In some embodiments, the phage or plasmid further comprises a polynucleotide encoding a guide RNA. In some embodiments, the phage or plasmid further comprises a polynucleotide encoding a Cas protein. In some embodiments, the Aca protein is substantially identical (e.g., at least 60%, 70%, 80%, 90%, 95% identical) to one of SEQ ID NOS: 1-27 or SEQ ID NOS: 50-60.

In another aspect, the present disclosure provides a bacterial cell comprising a polynucleotide encoding an anti-CRISPR associated (Aca) protein operably linked to a promoter, wherein the polynucleotide and/or the promoter is heterologous to the bacterial cell. In some embodiments, the bacterial cell further comprises a polynucleotide encoding a guide RNA. In some embodiments, the phage further comprises a polynucleotide encoding a Cas protein. In some embodiments, the Aca protein is substantially identical (e.g., at least 60%, 70%, 80%, 90%, 95% identical) to one of SEQ ID NOS: 1-27 or SEQ ID NOS: 50-60.

Numerous embodiments of the present invention, including compositions and methods for their preparation and administration, are presented herein.

### BRIEF DESCRIPTION OF THE DRAWINGS

FIG. 1. Anti-CRISPRs are found in diverse genomic contexts. Schematic representation of the genome context of diverse anti-CRISPR genes. Colored arrows represent anti-CRISPR and anti-CRISPR-associated (aca) genes as well as nearby genes encoding helix-turn-helix (HTH) motif proteins. Other genes are shown in gray and predicted functions are indicated when known. Arrows representing genes are not shown to scale. Int=integrase.

FIGS. 2A-2B. Anti-CRISPR AcrIF1 from phage JBD30 functions fully in the unrelated phage JBD44. FIG. 2A: Schematic representation of the genomic context of AcrIF1 from phage JBD30. The anti-CRISPR region (outlined red) was inserted into a transposon, which was used to randomly introduce the anti-CRISPR region into phage JBD44 by transposon mutagenesis. F and G encode phage head and tail morphogenesis proteins, respectively. I/Z encodes the protease/scaffold and T encodes the major head protein. FIG. 2B: Ten-fold dilutions of lysates from phage JBD44 and phage JBD44 carrying the JBD30 anti-CRISPR locus (JDB44::acr) were applied on lawns of CRISPR-Cas intact P. aeruginosa strain PA14 and CRISPR-Cas deleted PA14 (PA14ΔCRISPR) expressing a crRNA targeting phage JBD44 (JBD44 crRNA) from a plasmid. A representative image from three biological replicates is shown.

FIGS. 3A-3G. acrIF1 expression is driven by a promoter region that includes binding sites for Aca1. FIG. 3A: Relative levels of transcription of phage genes were measured by RT-qPCR at the indicated times after infection of strain PA14 by phage JBD30. Transcriptional levels are shown of the anti-CRISPR gene (acrIF1), an early expressed gene (A, transposase), and a late expressed gene (G, a tail component) during one round of phage infection at a multiplicity of infection (MOI) of 5. Levels were normalized to the geometric mean of the transcript levels of two host housekeeping genes: clpX and rpoD. The mean of three independent experiments is shown, with error bars representing standard error of the mean. FIG. 3B: Multiple nucleotide sequence alignment of anti-CRISPR phages from the stop codon of the Mu G homolog (G stop) to the start codon of the anti-CRISPR genes (acr start). Bioinformatically predicted promoter elements (BPROM; Solovyev and Salamov, 2011)-10 and -35 are shown. Inverted repeats are indicated by red boxes. A common inverted motif in both repeats is underlined. Positions sharing greater than 85% identity are colored according to nucleotide. FIG. 3C: The putative anti-CRISPR promoter region from phage JBD30 was cloned upstream of a promoterless lacZ expression vector (lacZ+acrIF1 upstream) and β-galactosidase activity was measured in P. aeruginosa strain PA14. The mean of three independent assays is shown, with error bars representing standard error of the mean.

FIG. 3D: Ten-fold dilutions of wild-type (JBD30), anti-CRISPR gene frameshift mutant (JBD30acrfs) and anti-CRISPR promoter mutant (JBD30ΔPacr) phage lysates were applied to lawns of CRISPR-Cas intact (PA14) and CRISPR-Cas deleted PA14 (PA14ΔCRISPR). A representative image from three biological replicates is shown. FIG. 3E: Electrophoretic mobility shift assays (EMSAs) were performed utilizing a fragment of dsDNA with the sequence shown, which encompasses the acr promoter region. The IR1 and IR2 mutants contained the triple and quadruple base substitutions indicated under the DNA sequence. Representative non-denaturing polyacrylamide gels stained with SYBR gold are shown. Purified Aca1 was added to the DNA at concentrations of 10 nM, 50 nM, 100 nM and 250 nM. The dash sign (−) indicates that no protein was added. FIG. 3F: The anti-CRISPR promoter region from phage JBD30 either wild-type (WT), or bearing IR1 and/or IR2 mutations was cloned upstream of a promoterless lacZ gene. β-galactosidase activity was measured in PA14 (−Aca1) or in a JBD30 lysogen (+Aca1). The mean β-galactosidase activity relative to the wild-type promoter is shown, with error bars representing standard error of the mean (n≥3). FIG. 3G: Ten-fold dilutions of lysates of anti-CRISPR phage JBD30 carrying the indicated inverted repeat mutations were applied to lawns of CRISPR-Cas intact PA14 or CRISPR deleted PA14 (PA14ΔCRISPR). Representative images from three biological replicates are shown.

FIGS. 4A-4E. Uncontrolled expression from the anti-CRISPR promoter is detrimental to phage viability. FIG. 4A: Representative electrophoretic mobility shift assays with indicated Aca1 mutants using the 110 bp upstream region from phage JBD30 as a substrate. Purified protein was added at concentrations of 10 nM, 50 nM, 100 nM, 250 nM and 500 nM. The dash sign (−) indicates that no protein was added. Non-denaturing acrylamide gels stained with SYBR gold are shown. FIG. 4B: The anti-CRISPR promoter region from phage JBD30 was cloned upstream of a promoterless lacZ gene. β-galactosidase activity was measured in a wild-type JBD30 lysogen (WT Aca1), JBD30 Aca1 mutant lysogens as indicated, and wild-type PA14 with no prophage (−). The mean from three independent experiments relative to the wild-type Aca1 JBD30 lysogen is shown, with error bars representing the standard error of the mean. FIG. 4C: Lysates of phage JBD30 (WT or Aca1R44A mutant) were spotted in 10-fold serial dilutions on bacterial lawns of wild-type P. aeruginosa PA14, a CRISPR deletion version of PA14 (PA14ΔCRISPR), or on the deletion strain bearing a plasmid that expresses Aca1. These phages are targeted by the CRISPR-Cas system in the absence of anti-CRISPR activity. Representative images from three biological replicates are shown. FIG. 4D: Lysates of wild-type JBD30, JBD30aca^{R44A} mutant phage and revertant JBD30acaR^{44A} phage were spotted in 10-fold serial dilutions on bacterial lawns of wild-type P. aeruginosa PA14 or on PA14ΔCRISPR. The sequence of the revertant phage demonstrating the loss of the −35 element from the anti-CRISPR promoter when compared to the sequence of the parent phage is shown below. FIG. 4E: The transcript levels of the acrIF1 and aca1 genes in phages bearing anti-CRISPR promoter operator mutants and aca mutants were determined by RT-qPCR. Expression levels were normalized to the geometric mean of the transcript levels of two bacterial housekeeping genes: clpX and rpoD. The mean is shown, with error bars representing the standard error of the mean (n=3). Assays were performed in PA14ΔCRISPR lysogens of the indicated phages.

FIGS. 5A-5D. Loss of Aca1 repressor activity affects the transcription of the gene immediately downstream of the anti-CRISPR locus. The transcription of the indicated phage genes from wild-type JBD30 and JBD30aca1^{R44A} during one-round of infection was determined by RT-qPCR. The genes assayed were: acrIFI (FIG. 5A); transposase, an early expressed gene (FIG. 5B); I/Z, the scaffold gene, which lies immediately downstream of aca1 (FIG. 5C); and G, a late gene lying directly upstream of the acr gene (FIG. 5D). Expression levels were normalized to the geometric mean of the transcript levels of two bacterial housekeeping genes: clpX and rpoD. The mean of three independent experiments is shown, with error bars representing the standard error of the mean. Assays were performed in PA14ΔCRISPR at a MOI of 8.

FIGS. 6A-6B. Overexpression of Aca1 inhibits phage-borne anti-CRISPRs. FIG. 6A: Tenfold dilutions of lysates of JBD30 carrying the indicated mutations in the Aca1 binding sites (IR1 and IR2) were applied to lawns of wild-type PA14 or PA14ΔCRISPR expressing wild-type Aca1 from a plasmid. A representative image from three biological replicates is shown. FIG. 6B: Ten-fold dilutions of lysates of anti-CRISPR phage JBD30 carrying the indicated inverted repeat mutation were applied to lawns of CRISPR-Cas intact PA14 or CRISPR deleted PA14 (PA14ΔCRISPR) expressing the R44A Aca1 mutant from a plasmid. A representative image from three biological replicates is shown.

FIGS. 7A-7C. Members of other Aca families are repressors of putative anti-CRISPR promoters. Promoter regions of acrIF1 (FIG. 7A) from Pseudomonas phage JBD30, and putative promoter regions of acrIF8 (FIG. 7B) from Pectobacterium phage ZF40, and acrIIC3 (FIG. 7C) from a N. meningitidis prophage were cloned upstream of a promoterless lacZ gene. β-galactosidase activity was measured in the absence and presence of the indicated Aca proteins expressed from a plasmid in E. coli. The cognate Aca for each promoter is underlined. The mean from three biological replicates is shown, with error bars representing the standard error of the mean.

FIGS. 8A-8D. Bioinformatic and functional analysis of Aca1. FIG. 8A: Multiple sequence alignment of Aca1 homologs from the indicated phages and bacteria. The position of the predicted helix-turn-helix (HTH) motif is outlined in a black box. Arrows indicate R33, R34, and R44, which were subjects of alanine substitution. FIG. 8B: Representative electrophoretic mobility shift assays with Aca1 using the 110-bp anti-CRISPR upstream region from phage JBD30 as a substrate. Purified protein was added at concentrations of 10 nM, 50 nM, 100 nM, 250 nM and 500 nM. The dash sign (−) indicates that no protein was added. Non-denaturing acrylamide gels stained with SYBR gold are shown. FIG. 8C: Quantification of DNA bound by Aca1 in electrophoretic mobility shift assays. Error bars represent the standard deviation of the mean of three replicates. FIG. 8D: To indicate their position relative to a DNA substrate, residues R33, R34, and R44 (highlighted in red) of JBD30 Aca1 were modeled onto the HTH DNA binding domain of the virulence regulator PlcR in complex with DNA (PDB: 3U3W) from Bacillus thuringiensis (Grenha et al., 2013).

FIGS. 9A-9B. Aca1 mutations alter phage plaque size, not viability. FIG. 9A: Ten-fold dilutions of lysates of the JBD30 phage carrying the indicated Aca1 mutation were applied to lawns of CRISPR intact PA14 (PA14) and CRISPR-deleted (PA14ΔCRISPR). A representative image from three biological replicates is shown. FIG. 9B: The plaque sizes (area) of the Aca1 partial DNA binding mutants in phage JBD30 were quantified on the PA14ΔCRISPR strain. The average size is shown relative to that of wild-type JB30 phage. Averages were calculated from three independent plaque assays, where >100 plaques were measured. Error bars represent the standard error of the mean. Representative plaque images are shown.

FIG. 10. Phage JBD30 lysogen formation is unaffected by the R44A Aca1 substitution. The PA14ΔCRISPR strain was infected with wild-type JBD30 (WT Aca1) or JBD30aca1R44A (R44A Aca1) at the same multiplicity of infection and plated to obtain single colonies. Lysogens were identified by cross-streaking the colonies over top of a line of phage lysate. The mean percentage of lysogens formed in three independent infection assays where 100 colonies were screened relative to the wild-type phage is shown, with error bars representing standard error of the mean.

FIGS. 11A-11D. Multiple sequence alignment of other Acas and their respective anti-CRISPR upstream regions. FIG. 11A: Multiple sequence alignment of Aca2 proteins from diverse Proteobacteria. The predicted helix-turn-helix motif is outlined in a black box. FIG. 11B: Multiple nucleotide sequence alignment of the region immediately upstream of the anti-CRISPR genes found in association with aca2 in panel A. A putative Aca2 binding site is outlined in a black box. Positions with >60% identity are colored. FIG. 11C: Multiple sequence alignment (MAFFT) of Aca3 proteins from different strains of Neisseria meningitidis. The predicted helix-turn-helix motif is outlined in a black box. FIG. 11D: Multiple nucleotide sequence alignment of the region immediately upstream of the anti-CRISPR genes found in association with aca3 in panel C. A putative binding site for Aca3 is outlined in a black box. Nme, Neisseria meningitidis; numbers indicate strain. Positions with >60% identity are colored.

FIGS. 12A-12G. FIG. 12A: AcrIIA1 NTD represses the deployment of anti-CRISPRs from phages. Four phages encoding Type II-A anti-CRISPRs were used to infect strains expressing AcrIIA1 FL (full length), the N-terminal domain (NTD), or no protein (EV) in backgrounds that contain (Cas9) or where it was knocked out, ΔCas9. Each phage replicates well in the absence of Cas9 or when the anti-CRISPR AcrIIA1 is expressed. In the presence of Cas9 EV, note that the phage with its anti-CRISPR deleted A006Δ is unable to replicate as well as the phage with the anti-CRISPR (A006) or where an anti-CRISPR is expressed in trans. Moreover, we observe that the expression of the AcrIIA1 NTD (which does not possess anti-CRISPR activity) actually limits the ability of anti-CRISPR phages to deploy their anti-CRISPRs. The A1-NTD impact is dependent on Cas9, consistent with inhibiting anti-CRISPR deployment and not another aspect of phage biology. FIG. 12B: Expression of the AcrIIA1 NTD can re-activate Cas9 that was inhibited by Acrs. A western blot is shown, measuring the level of Cas9 protein and a loading control in Listeria monocytogenes bacteria. In the absence of a prophage or any expressed protein, Cas9 is highly abundant (Lane 1). In lanes 2-4, a prophage is present in the strain, expressing the indicated anti-CRISPR locus, with AcrIIA1 and AcrIIA2. The expression of the AcrIIA1 anti-CRISPR causes the loss of Cas9 protein, and while EV or overexpression of A1-FL do not prevent this Cas9 loss, we observe (Lane 4) that overexpression of the A1-NTD reactivates Cas9 expression. This is due to the ability of the NTD to repress the anti-CRISPR promoter. This is not seen in the presence of A1-FL because the CTD of this protein is what mediates the Cas9 loss. FIG. 12C: Phage anti-CRISPR promoters are repressed by AcrIIA1-NTD. The promoter sequences of 5 distinct anti-CRISPR Listeria phages with the binding site highlighted in yellow. The panlindrome sequence is shown below the alignment and was fused to RFP as a reporter. In the reporter, RFP is well expressed from the anti-CRISPR promoter, but repressed in the presence of AcrIIA1-FL or just the A1-NTD. When the palindrome is mutated at two positions, AcrIIA1-FL is no longer able to repress its transcription. FIG. 12D: AcrIIA1 protein binds to the phage anti-CRISPR promoter. Raw data of a binding assay is shown, where the green line depicts the strong binding of AcrIIA1 protein to the phage anti-CRISPR promoter (34 nM binding constant). Mutations to the DNA sequence (depicted in red) weaken binding. FIG. 12E: Quantification of repressor activity of AcrIIA1 point mutants. The Acr promoter-RFP reporter construct was used to test AcrIIA1 mutants to confirm the important region of the protein responsible for DNA binding. This mutagenesis revealed key residues in the NTD required for function and also in the dimerization interface. FIG. 12F: Quantification of repressor activity of AcrIIA1 homologs. Homologs of AcrIIA1 are shown, with their % seq ID to the model protein from phage A006. The ability of the protein to repress their ‘cognate promoter’ (i.e. their own endogenous promoter) or the A006 promoter is quantified. Lastly, the ability of A006 AcrIIA1 to repress the promoters from the indicated elements are indicated. FIG. 12G: Key residues in the NTD of AcrIIA1 for DNA binding/repression. Protein alignment of AcrIIA1 NTD helix-turn-helix motif with key residues implicated in panel E highlighted. Note the horizontal line that depicts where the strong identity breaks, which also corresponds with lost ability of these proteins to repress the A006 promoter and vice versa.

FIGS. 13A-13D. Phages Require the AcrIIA1NTD (N-terminal Domain) for Optimal Replication. FIGS. 13A-13B: Left: Representative images of plaquing assays where Listeria phages were titrated in ten-fold serial dilutions (black spots) on lawns of Lmo10403s (gray background) lacking Cas9 (Δcas9) and encoding AcrIIA1NTD (Δcas9; IIA1NTD). Dashed lines indicate where intervening rows were removed for clarity. Right: Cas9-independent replication of isogenic ΦJ0161a or ΦA006 phages containing distinct anti-CRISPRs. Asterisk (*) indicates genes that contain the strong RBS associated with orfA in WT ΦA006, whereas unmarked genes contain their native RBS. Plaque forming units (PFUs) were quantified on Lmo10403s lacking cas9 (Δcas9, gray shaded bars) and expressing AcrIIA1NTD (Δcas9; IIA1NTD, black bars). Data are displayed as the mean PFU/mL of at least three biological replicates ±SD (error bars). See FIG. S1A for phage titers of additional ΦA006 phages. FIG. 13C: Top: Acr promoter mutations that suppress the ΦJ0161aΔIIA1-2 growth defect that manifests in the absence of AcrIIA1NTD. Bottom: Representative images of suppressor (Supp) phage plaquing assays conducted as in 13A-13B. FIG. 13D: Induction efficiency of ΦJ0161 prophages. Prophages were induced with mitomycin C from Lmo10403s:: ΦJ0161 lysogens expressing cis-acrIIA1 from the prophage Acr locus (WT) or lacking acrIIA1 (ΔIIA1-2) and trans-acrIIA1 from the bacterial host genome (+) or not (−). Plaque forming units (PFUs) were quantified on Lmo10403s lacking cas9 and expressing AcrIIA1NTD (Δcas9; IIA1NTD). Data are displayed as the mean PFU/mL after prophage induction of four biological replicates ±SD (error bars).

FIGS. 14A-14F. AcrIIA1NTD autorepresses the anti-CRISPR locus promoter. FIG. 14A: Alignment of the phage anti-CRISPR promoter nucleotide sequences denoting the −35 and −10 elements (gray boxes) and conserved palindromic sequence (yellow boxes). See FIG. S2A for a complete alignment of the promoters. FIG. 14B: Expression of RFP transcriptional reporters containing the wild-type (left) or mutated (right) ΦA006-Acr.-promoter in the presence of AcrIIA1 (IIA1) or each domain (IIA1NTD or IIA1CTD). Representative images of three biological replicates are shown. FIG. 14C: Quantification of the binding affinity (KD; boxed inset) of AcrIIA1 for the palindromic sequence within the acr promoter using microscale thermophoresis. ND indicates no binding detected. The nucleotide mutations (red letters) introduced into each promoter substrate are listed above the graph. Data shown are representative of three independent experiments. FIG. 14D: Repression of the ΦA006Acr.-promoter RFP transcriptional reporter by AcrIIA1ΦA006 mutant proteins. Data are shown as the mean percentage RFP repression in the presence of the indicated AcrIIA1 variants relative to controls lacking AcrIIA1 of at least three biological replicates ±SD (error bars). FIG. 14E: Nanoluciferase (NLuc) expression from the anti-CRISPR locus promoter in Listeria strains lysogenized with an ΦA006 reporter prophage (ΦA006acr::nluc) expressing AcrIIA1 (1) or AcrIIA1NTD (1N), in the presence of differing levels of Cas9: none (Δcas9), endogenous (PEND), overexpressed (PHYPER). Data are shown as the mean fold change in RLU (relative luminescence units) of three biological replicates, i.e., independent lysogens ±SEM (error bars). p-values: ***<0.001, ****<0.0001. FIG. 14F: Immunoblots detecting FLAG-tagged LmoCas9 protein and a non-specific (ns) protein loading control in Lmo10403s::V0161a lysogens or non-lyosgenic strains containing plasmids expressing AcrIIA1 (IIA1) or AcrIIA1NTD (IIA1NTD). Dashed lines indicate where intervening lanes were removed for clarity. Representative blots of at least three biological replicates are shown.

FIGS. 15A-15C. Autorepression is a General Feature of the AcrIIA1 Superfamily. FIGS. 15A-15B: Repression of RFP transcriptional reporters containing the ΦA006Acr.-promoter (gray bars) or cognate-AcrIIAlhomolog¬.-promoters (black bars) by the indicated AcrIIA1Homolog proteins (FIG. 15A) or AcrIIA1ΦA006 protein (FIG. 15B). Data are shown as the mean percentage RFP repression in the presence of the indicated AcrIIA1 variants relative to controls lacking AcrIIA1 of at least three biological replicates ±SD (error bars). The percent protein sequence identities of each homolog to the ΦA006AcrIIA1NTD are listed in (FIG. 15A). FIG. 15C: Top: Schematic of the wild-type (WT) and mutated AcrIIA1NTD binding site within the C-terminal protein coding sequence (CDS) of AcrIIA1LMO10. Bottom: Plaquing assays where the P. aeruginosa DMS3m-like phage JBD30 is titrated in ten-fold dilutions (black spots) on a lawn of P. aeruginosa (gray background) expressing the indicated anti-CRISPR proteins and Type II-A SpyCas9-sgRNA programmed to target phage DNA. Representative pictures of at least 3 biological replicates are shown.

FIGS. 16A-16E. AcrIIA1NTD Encoded from a Bacterial Host Displays “anti-anti-CRISPR” Activity. FIG. 16A: Schematic of host-AcrIIA1NTD homologs encoded in core bacterial genomes next to Type II-A, I-C, and I-E CRISPR-Cas loci in Lactobacillus delbrueckii strains. FIG. 16B: Seven promoters from the indicated phages and prophages were placed upstream of RFP, in the presence or absence of host-encoded AcrIIA1NTD, and fluorescence readout as in FIG. 3. FIG. 16C: Left panels: Plaquing assays where the indicated L. monocytogenes phages are titrated in ten-fold dilutions (black spots) on lawns of L. monocytogenes (gray background) expressing anti-CRISPRs from plasmids, LmoCas9 from a strong promoter (pHyper-cas9) or lacking Cas9 (Δcas), and the natural CRISPR array containing spacers with complete or partial matches to the DNA of each phage. (†) Denotes the absence of a spacer targeting the ΦJ0161a phage. Representative pictures of at least 3 biological replicates are shown. Right panel: Schematic of bacterial “anti-anti-CRISPR” activity where host-encoded AcrIIA1NTD (hA1NTD) blocks the expression of anti-CRISPRs from an infecting phage. FIG. 16D: Nanoluciferase (NLuc) expression from the anti-CRISPR locus promoter or a FIG. 16E: late viral promoter during lytic infection (Meile et al., 2020). L. monocytogenes 10403S strains expressing AcrIIA1 or AcrIIA1NTD from a plasmid were infected with reporter phages ΦA006acr::nluc or ΦA006 ΔLCR ply::nluc. Data are shown as the mean fold change in RLU (relative luminescence units) of three biological replicates ±SD (error bars).

FIG. 17. Optimal ΦA006 Phage Replication Requires AcrIIA1NTD, Related to FIG. 13. Left: Representative images of plaquing assays where the indicated Listeria phages were titrated in ten-fold serial dilutions (black spots) on lawns of Lmo10403s (gray background) lacking Cas9 (Δcas9) and encoding AcrIIA1NTD (Δcas9; IIA1NTD). Dashed lines indicate where intervening rows were removed for clarity. Right: Cas9-independent replication of isogenic ΦA006 phages containing distinct anti-CRISPRs. Asterisk (*) indicates genes that contain the strong RBS associated with orfA in WT ΦA006, whereas unmarked genes contain their native RBS. Plaque forming units (PFUs) were quantified on Lmo10403s lacking cas9 (Δcas9, gray shaded bars) and expressing AcrIIA1NTD (Δcas9; IIA1NTD, black bars). Data are displayed as the mean PFU/mL of at least three biological replicates ±SD (error bars). Note that this figure contains the same subset of data displayed in FIG. 13A.

FIGS. 18A-18B. AcrIIA1NTD Binds a Highly Conserved Palindromic Sequence in Acr Promoters, Related to FIG. 14. FIG. 18A: Alignment of the phage anti-CRISPR promoter nucleotide sequences denoting the −35 and −10 elements and ribosomal binding site (RBS) (gray boxes) and conserved palindromic sequence (yellow highlight). FIG. 18B: Quantification of DNA binding abilities (KD; boxed inset) of full-length AcrIIA1 and each domain (AcrIIA1NTD and AcrIIA1CTD) using microscale thermophoresis. Data shown are representative of three independent experiments. ND indicates no binding detected.

FIGS. 19A-19C. AcrIIA1 Homologs in Mobile Genetic Elements Across the Firmicutes Phylum Autoregulate their Cognate Promoters, Related to FIGS. 15, 16. FIG. 19A: Alignment of AcrIIA1 homolog protein sequences. FIG. 19B: Expression strength of the AcrIIA1 homolog promoters. Data are shown as the mean RFP expression (RFU normalized to OD600) driven by each AcrIIA1 homolog promoter of three biological replicates ±SD (error bars). FIG. 19C: Mobile genetic elements that possess an AcrIIA1 orthologue (red), which are either full-length or contain just the N-terminal domain (A1NTD). Arrows indicate the region corresponding to the promoter that was experimentally tested for repression by host-associated AcrIIA1NTD.

FIGS. 20A-20C. Bacterial expression of AcrIIA1NTD blocks phage anti-CRISPR deployment, Related to FIG. 16. FIG. 20A: Plaquing assays where the indicated L monocytogenes phages are titrated in ten-fold dilutions (black spots) on lawns of L. monocytogenes (gray background) expressing anti-CRISPRs from plasmids, LmoCas9 from a strong promoter (pHyper-cas9) or lacking Cas9 (Δcas9), and the natural CRISPR array containing spacers with complete or partial matches to the DNA of each phage. (†) Denotes the absence of a spacer targeting the ΦJ0161a phage. Representative pictures of 3 biological replicates are shown. Solid lines indicate where separate images are shown. FIG. 20B: Left panels: Plaquing assays where wild-type L. monocytogenes phages are titrated in ten-fold dilutions (black spots) on lawns of L. monocytogenes (gray background) containing single-copy integrated constructs expressing AcrIIA1 or AcrIIA1NTD from the ΦA006 anti-CRISPR promoter (pA006), LmoCas9 from a constitutive promoter (pHyper-Cas9), and the natural CRISPR array containing spacers with complete or partial matches to the DNA of each phage. (†) Denotes the absence of a spacer targeting the virulent phage ΦP35. Representative pictures of 3 biological replicates are shown. Right panel: Schematic of bacterial “anti-anti-CRISPR” activity where host-encoded AcrIIA1NTD (hA1NTD) blocks the expression of anti-CRISPRs from an infecting phage. FIG. 20C: Nanoluciferase (NLuc) expression from the anti-CRISPR locus promoter of an ΦA006 reporter phage ΦA006acr::nluc) during lytic infection of L. monocytogenes EGDe. Data are shown as the mean fold change in RLU (relative luminescence units) of three biological replicates ±SD (error bars).

FIGS. 21A-21B. FIG. 21A: Growth curves of PAO1IC lysogenized by recombinant DMS3m phage expressing acrIIA4 or acrIC1 from the native acr locus. CRISPR-Cas3 activity is induced with either 0.5 mM (+) or 5 mM (++) IPTG and 0.1% (+) or 0.3% (++) arabinose. Edited survivors reflect number of isolated survivor colonies missing the targeted gene (phzM). Each growth curve is the average of 10 biological replicates and error bars represent SD. FIG. 21B: Genotyping results of PAO1IC AcrC1 lysogens after self-targeting induction in the presence or absence of aca1 and a non-targeted control. Ten biological replicates per strain were assayed. gDNA was extracted from each replicate and PCR analysis for the phzM gene (targeted gene, top row of gels) or cas5 gene (non-targeted gene, bottom row) was conducted. Only cells that co-expressed aca1 with the crRNA showed loss of the phzM band, indicating genome editing. All replicates had a cas5 band, indicating successful gDNA extraction and target specificity for the phzM locus.

### Definitions

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter. In particular embodiments, the promoter is a prokaryotic promoter, e.g., a promoter used to drive aca gene expression in prokaryotic cells. Typical prokaryotic promoters include elements such as short sequences at the −10 and −35 positions upstream from the transcription start site, such as a Pribnow box at the −10 position typically consisting of the six nucleotides TATAAT, and a sequence at the −35 position, e.g., the six nucleotides TTGACA.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of an Aca can have an increased stability, assembly, or activity as described herein.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215: 403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of eubacterial and archaeal organisms. CRISPR-Cas systems fall into two classes with six types, I, II, III, IV, V, and VI as well as many sub-types, with Class 1 including types I and III CRISPR systems, and Class 2 including types II, IV, V and VI; Class 1 subtypes include subtypes I-A to I-F, for example. See, e.g., Fonfara et al., Nature 532, 7600 (2016); Zetsche et al., Cell 163, 759-771 (2015); Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9. Endogenous systems function with two RNAs transcribed from the CRISPR locus: crRNA, which includes the spacer sequences and which determines the target specificity of the system, and the transactivating tracrRNA. Exogenous systems, however, can function which a single chimeric guide RNA that incorporates both the crRNA and tracrRNA components. In addition, modified systems have been developed with entirely or partially catalytically inactive Cas proteins that are still capable of, e.g., specifically binding to nucleic acid targets as directed by the guide RNA, but which lack endonuclease activity entirely, or which only cleave a single strand, and which are thus useful for, e.g., nucleic acid labeling purposes or for enhanced targeting specificity. Any of these endogenous or exogenous CRISPR-Cas system, of any class, type, or subtype, or with any type of modification, can be utilized in the present methods. In particular, “Cas” proteins can be any member of the Cas protein family, including, inter alia, Cas3, Cas5, Cas6, Cas7, Cas8, Cas9, Cas10, Cas12 (including Cas12a, or Cpf1), Cas13, Cse1, Cse2, Csy1, Csy2, Csy3, GSU0054, Csm2, Cmr5, Csx11, Csx10, Csf1, Csn2, Cas4, C2c1, C2c3, C2c2, and others. In particular embodiments, Cas proteins with endonuclease activity are used, e.g., Cas3, Cas9, or Cas12a (Cpf1).

“Anti-CRISPR” (Acr) elements refer to loci from phage, plasmids, prophages, conjugative islands, and other mobile genetic elements, as well as the polypeptides that they encode, that are capable of inhibiting endogenous or exogenous CRISPR-Cas systems. See, e.g., Borges et al. 2018; Rauch et al., 2017; Bondy-Denomy et al,. 2013; Pawluk et al., 2016b. Anti-CRISPR proteins are typically small (approximately 50-150 amino acids) and function, e.g., by preventing CRISPR-Cas systems from recognizing foreign nucleic acids or by inhibiting their nuclease activity. Acr proteins display no common features with respect to sequence, predicted structure, or genomic location of their encoding genes. A wide variety of Anti-CRISPRs have been identified, from a diversity of viruses and other mobile elements, showing a tremendous amount of sequence diversity, with 40 distinct families now identified. Acrs can be identified in various ways known to those of skill in the art, e.g., by virtue of sequence homology to known Acrs, via the detection of protospacers (i.e., sequences complementary to natural spacers in the CRISPR array in prophage sequences, which is indicative of Acr activity in the cell), or by assays involving the introduction of plasmid-based protospacers and the measurement of transformation efficiency (see, e.g., Rauch et al. 2018).

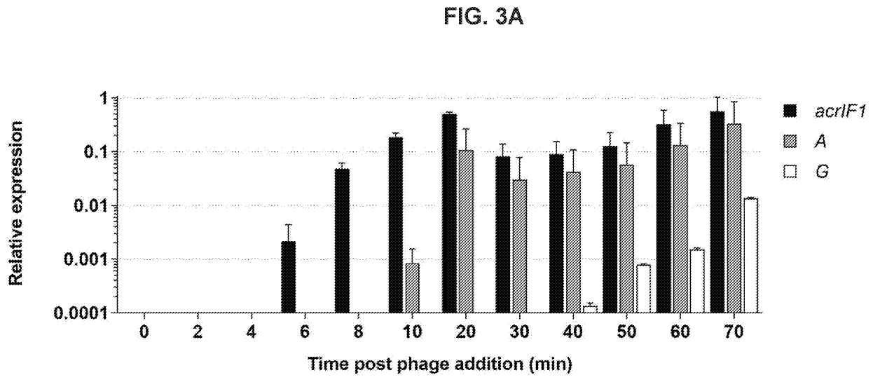

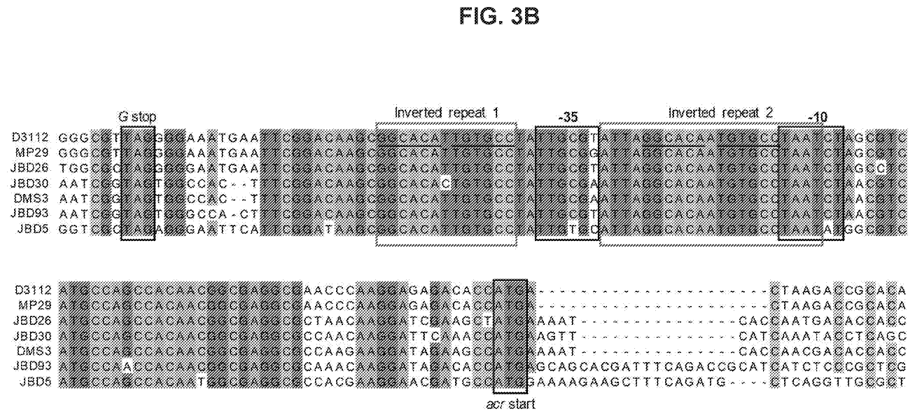

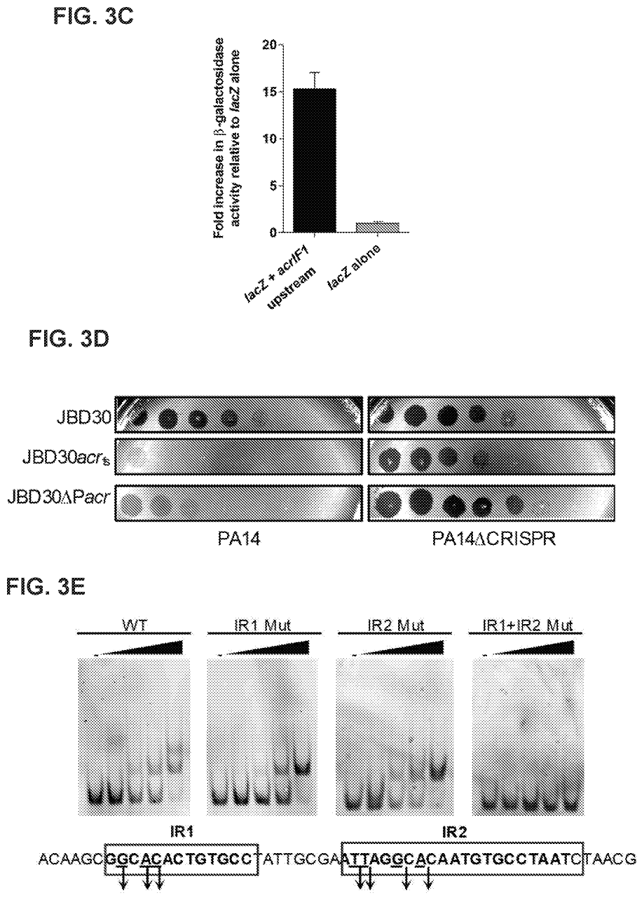

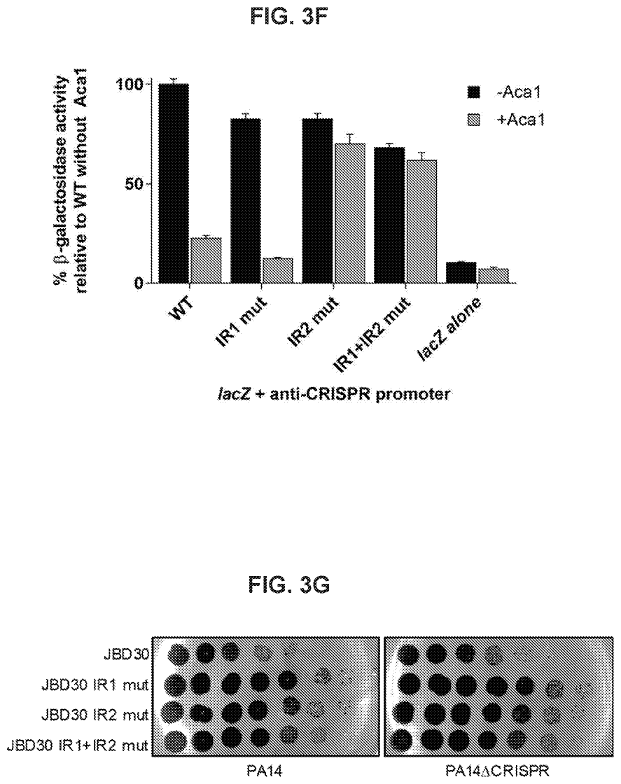

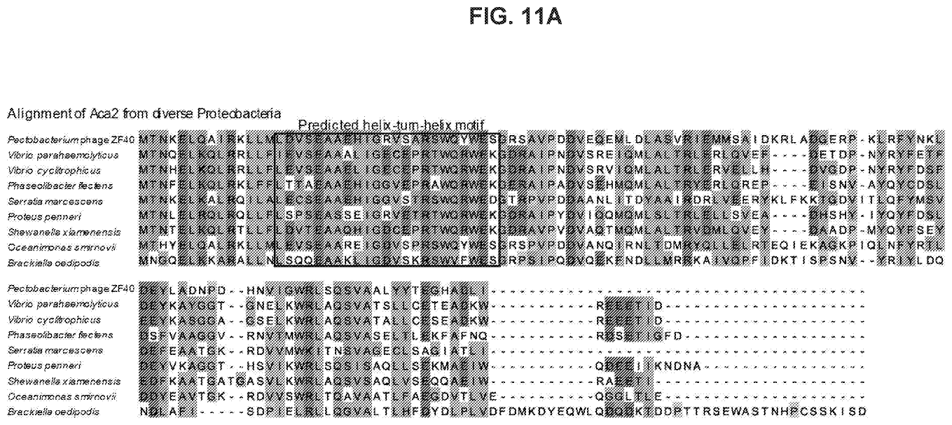

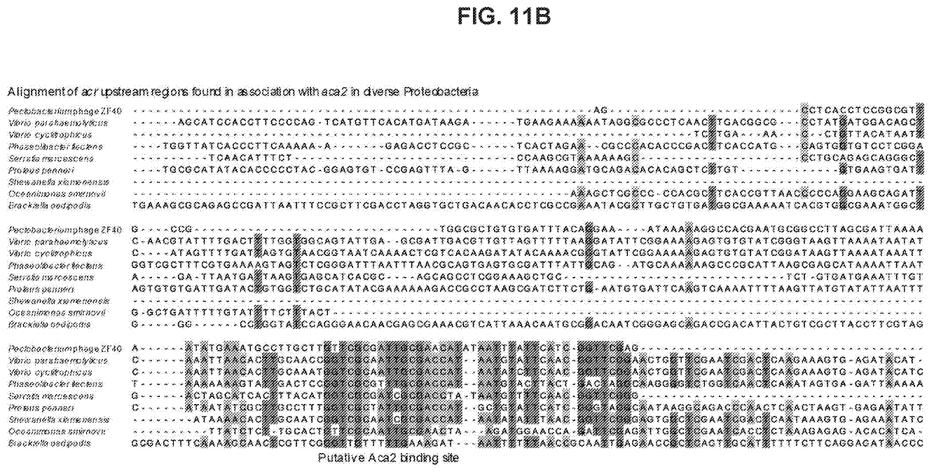

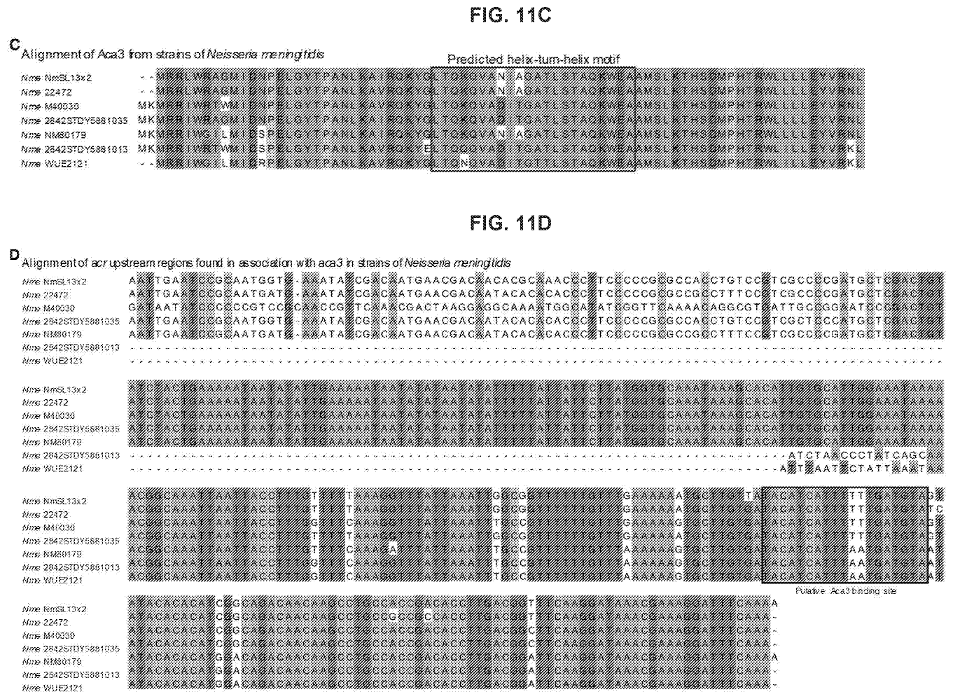

A feature of acr genes that is relevant to the present methods and that can be used for their identification is that they are virtually always associated with downstream “aca” genes encoding Helix-Turn-Helix (HTH)-containing “anti-CRISPR associated” (Aca) proteins, which bind to the promoters of the acr genes and inhibit their expression. “Acr promoters,” which are promoters as defined herein that control transcription of acr genes, typically contain one or more inverted repeats, which can be bound by Aca proteins. Examples of acr promoters include SEQ ID NOS. 28-49, or as shown in, e.g., FIG. 3 or 11, but it will be understood that any acr promoter, from any species and controlling any acr coding sequence, that can be bound by an Aca protein can be used in the present methods.

“Anti-CRISPR-associated” (Aca) proteins, or (aca) genes, refers to a family of genes and encoded proteins that are associated with, e.g., downstream of within the same operon, Anti-CRISPR loci. Aca proteins contain Helix-Turn-Helix (HTH) domains and bind to acr promoters, typically to the inverted repeats within acr promoters, and repress transcription of the acr coding sequence. Acas include, but are not limited to, Aca1, Aca2, Aca3, Aca4, Aca5, Aca6, Aca7, Aca8, or AcrIIA1 family members, variants, derivatives, or fragments, e.g., the NTD domain, thereof from any species, as presented in the Examples, Tables, and Figures, and SEQ ID NOS. 1-27 and 50-60, as well as polynucleotides sharing at least 50%, 60%, 70%, 80%, 90%, 95%, 96%, 97%, 98%, 99%, to any of SEQ ID NOS. 1-27 or 50-60 or of any of the Acas shown in the Tables or Figures. It will be understood that any aca gene associated with any acr locus from any species, i.e., a sequence coding for an HTH-containing polypeptide that is capable of binding to the acr locus and inhibiting its transcription, is encompassed by the present methods.

### DETAILED DESCRIPTION OF THE INVENTION

The inventors have discovered that Anti-CRISPR-Associated (Aca) proteins act to inhibit the expression of Anti-CRISPR (Acr) proteins in prokaryotic cells. Accordingly, methods for introducing or enhancing Aca activity in prokaryotic cells have been discovered, for example to inhibit any known or potential Acr activity in the cells and thereby permit or enhance endogenous or exogenous CRISPR-Cas activity. Cells, polynucleotides, plasmids, phage, and other elements for practicing the present methods are also provided.

In some embodiments, a human or non-human mammalian or avian individual with a bacterial infection involving “self-targeting” bacteria, i.e., CRISPR-Cas-containing bacteria in which a spacer sequence within the CRISPR array matches a sequence present within the bacterial chromosome, indicating that an Acr is actively inhibiting the CRISPR-Cas system in the cells, is administered, e.g., using phage or via bacterial conjugation, a polynucleotide encoding an Aca operably linked to a promoter. In such embodiments, the polynucleotide will enter the bacterial cells and express the Aca at a level in the cells that is sufficient to inhibit the expression of the Acr in the cells, resulting in the activation of the CRISPR-Cas system, the Cas-mediated cleavage of the chromosome at the matching sequence, and the killing of the cells.

In some embodiments, an Aca protein is introduced into a prokaryotic cell expressing an Acr protein, wherein the Aca represses expression of the Acr protein and thereby allows the activation of the CRISPR-Cas system in the cell. In some embodiments, the Aca is introduced by introducing a polynucleotide encoding the Aca. In some embodiments, the Aca is introduced together with a guide RNA and/or a Cas protein (e.g., a polynucleotide encoding the Cas protein).

In another set of embodiments, an individual (e.g., as described above) with a bacterial infection is administered, e.g., using phage or via bacterial conjugation, a polynucleotide encoding an Aca, operably linked to a promoter, as well as a polynucleotide providing CRISPR-Cas activity (e.g., a Cas9 polynucleotide and a guide RNA specific to the infectious bacteria). In such embodiments, the polynucleotides will enter the infectious bacteria, resulting in the presence of Cas endonuclease activity in the cells that is specific to the bacteria and that is uninhibited by Acr activity, and in the cleavage of the target sequence complementary to the guide RNA and the destruction of the cells.

In another set of embodiments, an Aca protein and a CRISPR-Cas ribonucleoprotein are introduced into prokaryotic cells in vitro, e.g., by introducing polynucleotides encoding the protein and ribonucleoprotein by phage-mediated transduction, by transformation, or by bacterial conjugation, so as to obtain non-Acr-inhibited CRISPR-Cas activity in the cells, e.g., for genomic editing purposes, regulation of gene expression through CRISPR interference (CRISPRi) or CRISPR activation (CRISPRa), or for labeling purposes.

The cells targeted in the present methods can be any prokaryotic cells, including bacteria or archaea, in vitro or in vivo, that are suspected to, known to, or that potentially contain an Acr-encoding gene, and in which CRISPR-Cas activity is desired for any reason. Such cells could be, for example, undesired, self-targeting bacterial cells in which an Acr is preventing an endogenous CRISPR-Cas system from cleaving a prophage sequence that matches a spacer sequence in the CRISPR locus; in such cells, the methods could be used to activate the endogenous CRISPR-Cas in the cells and thereby kill the cells. The cells could be antimicrobial resistant bacteria in which a guide RNA can be introduced to target the antimicrobial resistance (AMR) locus and thereby selectively kill the cells or eliminate AMR-containing plasmids. The cells could be, e.g., undesired cells, and a guide RNA that is specific to a sequence in the cells' genomic DNA is introduced, so that the cells' genomic DNA is cleaved in the presence of CRISPR-Cas activity, thereby killing the cells. The cells could be strains in which CRISPR-Cas is desired in order to repress or activate the expression of a specific gene, e.g., using CRISPR interference (CRISPRi) or CRISPR activation (CRISPRa), or in which CRISPR-Cas is used for genome editing, e.g., for inducing deletions, insertions, or other modifications in a given gene of interest, or in which labeled Cas proteins are used for nucleic acid labeling, painting, or imaging. In all of these embodiments, in addition to the introduction of the Aca, the method may further comprise introducing other elements of the CRISPR-Cas system into the cells, e.g., one or more guide RNAs or one or more Cas proteins, for example by introducing a polynucleotide encoding the Cas protein or proteins.

The choice of Aca protein(s) to be introduced into the cell can depend on the cell type (e.g., genus or species) and the Acrs and Acas that are known to be or that are possibly present in the cell. Acas are naturally associated with one or more Acrs, as aca genes are present within acr operons in phage and prophage and their products (i.e., the Aca proteins) bind to and repress transcription from the acr promoters. For example, in the Pseudomonas aeruginosa phage JBD30, Aca1 is found in association with the acrIF1 gene, and with many other acr genes. Aca2 proteins are found in association with five different families of acr genes in diverse species of Proteobacteria, including with the AcrIF8 gene from the Pectobacterium phage ZF40, and Aca3 has been identified in association with three different type II-C Acrs, including with the AcrIIC3 gene from N. meningitides strain 284STDY5881035. In general, as each acr gene has an associated aca gene and as its expression is repressed by the Aca protein encoded by the associated gene, performing the present methods will be a matter of identifying the Acrs that are known to be or that are potentially present in the bacteria in question, and introducing one or more Acas that are capable of repressing the expression of the acr gene. In some embodiments, the Aca used is that encoded by the aca gene within the same operon as the acr gene. It will be appreciated, however, that any Aca polypeptide can be used, so long as it is capable of binding to and repressing transcription from an acr promoter that is present, or potentially present, in the cell. A non-limiting list of Acas, together with their associated Acrs and species information, that can be used in the present methods is provided as Tables 8 and 9, and are also provided in, e.g., FIGS. 3, 11, and 12, and in SEQ ID NOs: 1-27 and 50-60.

Any number of Acas can be used at a time for the purposes of the present methods. For example, a single Aca can be introduced into a cell to inhibit the expression of one or more acr genes. It will be appreciated, however, that multiple (e.g., 2, 3, 4, 5, or more) Acas can be used in series or simultaneously, e.g. introducing Acas corresponding to every potential Acr within a given cell type.

In many cases, simply knowing the genus or species of bacteria or prokaryotic cell to be targeted will be sufficient to allow the selection of the Aca(s) to be used, as it will be known which Acrs are potentially present in the genus or species in question, or within phage or other mobile genetic elements liable to infect or be present within cells of the genus or species. It will be appreciated, however, that it is not necessary to know whether or not the given cell type contains any Acrs and/or Acas, or what type of Acrs or Acas it contains, in order to perform the present methods. By providing one or more Acas to a targeted cell, e.g., alone or together with one or more guide RNAs and one or more Cas proteins, it is possible to target all known or potentially present Acrs in the cell, thereby ensuring the activation of the CRISPR-Cas system. In certain embodiments, plasmids will be created for use in particular bacterial genera or species that contain one or more Aca-encoding polynucleotides specific to acr genes liable to be present in the given cell type. Such plasmids are provided, as are phagemids, phage, and bacteria comprising the plasmids.

In certain embodiments, to complement existing knowledge about the Acas liable to be effective in a given cell type, the cells to be targeted can first be characterized with respect to the Acr and/or Aca proteins that they express, in order to provide additional guidance regarding the Aca polypeptides that may be used. For example, a sample of the cells to be targeted could be isolated and any acr or aca genes identified within the bacterial chromosome and/or plasmids, phage, or other mobile genetic sequences, e.g., by sequencing, by performing PCR-based assays, by querying appropriate sequence databases (e.g., NCBI), etc., for example using coding sequences or regulatory, e.g., promoter, sequences. In other embodiments, Acr proteins could be identified, e.g., using antibody-based assays. In some embodiments, the presence of anti-CRISPR activity in the cells can be assessed, e.g., using assays in which plasmids with protospacers are introduced into the cells and transformation efficiencies assessed (see, e.g., Rauch et al., 2017).

Once an acr gene or Acr protein has been identified in the cells, an appropriate Aca could be selected based on a known or suspected ability to bind to and repress the acr gene. In many cases, the Aca will be encoded by the aca gene present within the same operon as the acr gene in question, but it will be recognized by one of skill in the art that any Aca protein that is capable of binding to the acr promoter in question, e.g., through an inverted repeat in the promoter, and repressing its expression can be used.

As aca genes are strongly conserved and are virtually always found in association with acr genes, in certain embodiments it will be useful to directly identify the aca genes or Aca proteins present in the cells to be targeted. This can be done by virtue of their sequence conservation, e.g., within the Helix-Turn-Helix (HTH) domain, using bioinformatics approaches with sequence databases and/or or by sequencing the bacterial genome, prophage sequences, plasmids, or other mobile genetic sequences and searching for homology to known acas. If an aca gene or Aca protein is identified, it is likely that Acr proteins are present as well that are actively or potentially inhibiting CRISPR-Cas systems within the cells. In such cases, the identified Aca can be introduced into the cell so as inhibit the expression of the Acr and thereby bring about an increase in CRISPR-Cas activity.

Acrs have been identified to date in a wide variety of prokaryotic species, and it has been hypothesized that virtually all CRISPR-Cas systems, which are thought to be present in around 50% of all bacterial species, can be targeted by one or more Acrs. Accordingly, strategies to use endogenous or exogenous CRISPR-Cas to bring about, e.g., targeted destruction of cells, genomic modifications, alterations in gene regulation, and/or genomic labeling or painting, will likely be frequently impeded by the presence of Acrs in the cells. As such, the present method may be of widespread utility, and it will be useful to systematically include Aca-encoding polynucleotides in any plasmids destined to be used in CRISPR-Cas-based strategies in prokaryotic cells.

The present methods can be practiced with any Aca polypeptide, or any variant, derivative, or fragment, e.g., an N-terminal domain, or NTD, of an Aca polypeptide, so long that it is capable of binding to an acr promoter of interest and inhibiting its expression. Non-limiting examples of Aca sequences are shown in Tables 8 and 9 and are also presented below as SEQ ID NOS. 1-27 and SEQ ID NOS: 50-60:

(Aca1, Pseudomonas aeruginosa phage JBD30)
SEQ ID NO. 1
MRFPGVKTPDASNHDPDPRYLRGLLKKAGISQRRAAELLGLSDRVMRYY
LSEDIKEGYRPAPYTVQFALECLANDPPSA
(Aca1, Pseudomonas aeruginosa)
SEQ ID NO. 2
MQLKPRNTVPRPDASSHNPDPRYLRGLLKKAGISQRRAAELLGLGDRVM
RYYLSEDAKDGYRPAPYTVQFALECLANDPPSA
(Aca1, Pseudomonas otitidis)
SEQ ID NO. 3
MKPDASNHNPDPRYLRELIERAGVSQRQAAELIGMSWEGFRRYLRDVDA
PGYRVADYRVQFALECLAAPGT
(Aca1, Pseudomonas delhiensis)
SEQ ID NO. 4
MPLQQRSTVRKPDASNHNPNPRYLRGLVERSGKSQRQAAELLGLSWEGF
RNYLRDESHPLHRSAPYTVQFALECLAEAE
(Aca1, Pseudomonas aeruginosa)
SEQ ID NO. 5
MKPDSSKHNPDPQYLRGLYERAGLKQEEAARRIGITARALRNYVSETAG
REAPYPVQFALECLASES
(Aca2, Pectobacterium phage ZF40)
SEQ ID NO. 6
MTAMKEWRARMGWSQRRAAQELGVTLPTYQSWEKGIRLSDGSPIDPPLT
ALLAAAAREKGLPPIS
(Aca2, Vibrio parahaemolyticus)
SEQ ID NO. 7
MPLLFRSFIMTNQELKQLRRLLFIEVSEAAALIGECEPRTWQRWEKGDR
AIPNDVSREIQMLALTRLERLQVEFDETDPNYRYFETFDEYKAYGGTGN
ELKWRLAQSVATSLLCETEADKWREEETID
(Aca2, Shewanella xiamenensis)
SEQ ID NO. 8
MTNTELKQLRTLLFLDVTEAAQHIGDCEPRTWQRWEKGDRAVPVDVAQT
MQMLALTRVDMLQVEYDAADPMYQYFSEYEDFKAATGATGASVLKWRLA
QSVSAQLVSEQQAEIWRAEETI
(Aca2, Brackiella oedipodis)
SEQ ID NO. 9
MNGQELKKARALLNLSQQEAAKLIGDVSKRSWVFWESGRPSIPQDVQEK
FNDLLMRRKAIVQPFIDKTISPSNVYRIYLDQNDLAFISDPIELRLLQG
VALTLHFDYDLPLVDFDMKDYEQWLQDQDKTDDPTTRSEWASTNHPCSS
KISD
(Aca2, Oceanimonas smirnovii)
SEQ ID NO. 10
MTHYELQALRKLLMLEVSEAAREIGDVSPRSWQYWESGRSPVPDDVANQ
IRNLTDMRYQLLELRTEQIEKAGKPIQLNFYRTLDDYEAVTGKRDVVSW
RLTQAVAATLFAEGDVTLVEQGGLTLE
(Aca3, Neisseria meningitidis 2842STDY5881035)
SEQ ID NO. 11
MKMRRIWRAGMIDNPELGYTPANLKAIRQKYGLTQKQVADITGATLSTA
QKWEAAMSLKTHSDMPHTRWLLLLEYVRNL
(Aca4, Pseudomonas aeruginosa)
SEQ ID NO. 12
MTPDQFDALAELIRLRGGASQEAARLVLVDGMSPSDAARQVEASPQAVS
NVLASCRRGLALVLRASGKGATA
(Aca4, Pseudomonas aeruginosa)
SEQ ID NO. 13
MTKEQFSALAELMRLRGGPGQDAARLVLVNGLIKPTEAARQTGITPQAV
NKTLSSCRRGIELAKRVFT
(Aca4, Pseudomonas stutzeri)
SEQ ID NO. 14
MMTGEQFGALAELLRLRGGASQEAARLVLVEGLAPAEAARQAGTTPQAV
SNALASCRRGLELARVAAG
(Aca4, Pseudomonas sp.)
SEQ ID NO. 15
MTAEQFSALAELLRLRGGASQEAARLVLVEQLTPAEAARAAGCSPQAVS
NVLASCRRGLELAHAAVGH
(Aca5, Yersinia frederiksenii)
SEQ ID NO. 16
MPLIEYIRLTFSGNKSEFARHMGVDRQKVQVWIKGEWIVVGNKLYAPRR
DIPDIRLDTVSQRLD
(Aca5, Escherichia coli)
SEQ ID NO. 17
MNKMNARTLSDYIAFYHNGNQAEFARHMGVNRQQVTKWIKGGWIVINHQ
LFSPQRDIPENISHGGSAL
(Aca5, Serratia fonticola)
SEQ ID NO. 18
MNNDNLVSGRTLLGYINIFHNGSQADFARHMDVTPQQVTKWISGEWIVV
NHQLFSPKRDVPENISGGESAGN
(Aca5, Dickeya solani)
SEQ ID NO. 19
MKLSEFIDTEFSGSRAEFARLMGVRPQKVNDWLVAGMIIHIDENGQAFL
CSVRRDIPAWNRKTNFA
(Aca5, Pectobacterium carotovorum)
SEQ ID NO. 20
MSLTEYIDKNFGGNKAAFARHMGVDAQAVNKWIKSEWFVSTTDDNKIYL
SSARREIPPLK
(Aca5, Enterobacter cloacae complex)
SEQ ID NO. 21
MNARTLSDYIEFYHNGNQSDFARHMGVNRQQVTKWLNGGWVVINHQLYS
PQRDVPEFVTGGGSAL
(Aca5, Pectobacterium carotovorum)
SEQ ID NO. 22
MSLTEYIDKNFAGNKAAFARHMGVDAQAVNKWIKSEWFVSTTDDNKIYL
SSVRREIPPVA
(Aca6, Alcanivorax sp.)
SEQ ID NO. 23
MTAMKEWRARMGWSQRRAAQELGVTLPTYQSWEKGIRLSDGSPIDPPLT
ALLAAAAREKGLPPIS
(Aca6, Alcanivorax sp.)
SEQ ID NO. 24
MTAMKDWRTRMGWSQRRAAQELGVTLPTYQSWERGVRLSDGSLIDPPLT
ALLAAAAREKGLDPI
(Aca7, Halomonas caseinilytica)
SEQ ID NO. 25
MIDARKHYDPNLAPELVRRALAVTGTQKELAERLDVSRTYLQLLGKGQK
SMSYAVQVMLEQVIQDGET
(Aca7, Halomonas sinaiensis)
SEQ ID NO. 26
MIDARKYYNPDLAPELVSRALAVTGTQKELAERLDVSRIYIQLLGKGQK
TMSYAVQVMLEQVIQGGEN
(0d13, Cryptobacterium curtum)
SEQ ID NO. 27
MPIKDLTGMRFGRLVVKEATSRRTSDGNVIWRCQCDCGNVTEVPGHSLT
RGNTRSCGCGEEENRRESGNNRNKAVVKEHSRADSFLSPKPRADTTLGI
RGILRRPSGRYAARITFKGKTTCLGTYDSLEEAANARREAEIEIFDPYL
IANGLPPTSEEEWQKILARALEKEKDNADTSTKARPGKIRARKNKAVQN

The Acas that can be used will include those comprising SEQ ID NOS: 1-27 and SEQ ID NOS: 50-60 and as shown in Tables 8 and 9 and in the Figures, as well as variants, derivatives, fragments, and homologs thereof having at least 50%, 55%, 60%, 65%, 70%, 75%, 80%, 85%, 90%, 95%, 96%, 97%, 98%, 99%, or greater identity to any of SEQ ID NOS: 1-27 of 50-60, and/or the Aca sequences shown in Tables 8 or 9 and in the Figures. Variants, derivatives, and fragments can be readily assessed using standard biochemistry assays for their ability to bind to the acr promoter sequences, e.g., to inverted repeats within acr promoters, and to inhibit transcription as assessed, e.g., using qRT-PCR assays.

Non-limiting examples of acr promoter sequences that can be targeted in the present methods and that can be used in the assays described herein include the sequences provided herein as SEQ ID NOS 28-49, the sequences provided in the Figures, e.g., FIGS. 3 and 11, as well as variants and homologs thereof having at least 50%, 55%, 60%, 65%, 70%, 75%, 80%, 85%, 90%, 9%, 96%, 97%, 98%, 99%, or greater identity to any of SEQ ID NOS 28-49 or of the sequences provided in the Figures, e.g., FIGS. 3 and 11.

Aca1-associated acr promoter sequences
(D3112 acrIF1 promoter)
SEQ ID NO. 28
GGGCGTTAGGGGAAATGAATTCGGACAAGCGGCAC
ATTGTGCCTATTGCGTATTAGGCACAATGTGCCTA
ATCTAGCGTCATGCCAGCCACAACGGCGAGGCGAA
CCCAAGGAGAGACACCATGA
(MP29 acrIF1 promoter)
SEQ ID NO. 29
GGGCGTTAGGGGAAATGAATTCGGACAAGCGGCAC
ATTGTGCCTATTGCGGATTAGGCACAATGTGCCTA
ATCTAGCGTCATGCCAGCCACAACGGCGAGGCGAA
CCCAAGGAGAGACACCATGA
(JBD26 acrIF1 promoter)
SEQ ID NO. 30
TGGCGCTAGGGGGAATGAATTCGGACAAGCGGCAC
ATTGTGCCTATTGCGTATTAGGCACAATGTGCCTA
ATCTAGCCTCATGCCAGCCACAACGGCGAGGCGCT
AACAAGGATCGAAGCTATGA
(JBD30 acrIF1 promoter)
SEQ ID NO. 31
AATCGGTAGTGGCCACTTTCGGACAAGCGGCACAC
TGTGCCTATTGCGAATTAGGCACAATGTGCCTAAT
CTAACGTCATGCCAGCCACAACGGCGAGGCGCCAA
CAAGGATTCAAACCATGA
(DMS3 acrIF1 promoter)
SEQ ID NO. 32
AATCGGTAGTGGCCACTTTCGGACAAGCGGCACAT
TGTGCCTATTGCGAATTAGGCACAATGTGCCTAAT
CTAACGTCATGCCAGCCACAACGGCGAGGCGCCAA
GAAGGATAGAAGCCATGA
(JBD93 acrIF1 promoter)
SEQ ID NO. 33
AATCGGTAGTGGGCCACTTTCGGACAAGCGGCACA
TTGTGCCTATTGCGTATTAGGCACAATGTGCCTAA
TCTAACGTCATGCCAACCACAACGGCGAGGCGCAA
ACAAGGATAGACACCATGA
(JBD5 acrIF1 promoter)
SEQ ID NO. 34
GGTCGCTAGAGGGAATTCATTCGGATAAGCGGCAC
ATTGTGCCTATTGTGCATTAGGCACAATGTGCCTA
ATATGGCGTCATGCCAGCCACAATGGCGAGGCGCC
ACGAAGGAACGATGCCATGG
Aca2 associated acr promoter sequences
(Phaseolibacter flectens)
SEQ ID NO. 35
TGGTTATCACCCTTCAAAAAAGAGACCTCCGCTCA
CTAGAACGCCCACACCCGACTTCACCATGCAGTGG
TGTCCTCGGAGGTCGCTTTCGTGAAAAGTAGTCTC
GGGATTTAATTTAACGCAGTGAGTGCGATTTATTG
CAGATGCAAAAAAGCCCGCATTAAGCGAGCATAAA
ATTAATTAAAAAAAGTATTGACTCCGGTCGCGTTT
GCGACCATAATGTACTTACTGACTAGGCAAGGGGT
CTGGTCAACTCAAATAGTGAGATTAAAAA
(Proteus penneri)
SEQ ID NO. 36
TGCGCATATACACCCCCTACGGAGTGTCCGAGTTT
AGTTAAAAGGATGCAGACACACAGCTCTTGTGTGA
AGTGATTAGTGTGTGATTGATACTGTGGTCTGCAT
ATACGAAAAAAGACCGCCTAAGCGATCTTCTGAAT
GTGATTCAAGTCAAAATTTTAAGTTATGTATATTA
ATTTCATAATATCGCTTGCCTTTGGTCGCTATTGC
GACCATGCTGTATTCATCGGGTAGGCAATAAGGCA
GACCCAACTCAACTAAGTGAGAATATTA
(Shwanella xiamenensis)
SEQ ID NO. 37
ATAAAACACTTGCAATCGGTCGCAATCGCGACCAT
AATATATTTAACGGTTCGGGAGTGGCTCGAATCGA
CTCAATAAAGTGAGAAATATCA
(Vibro parapaemolyticus)
SEQ ID NO. 38
AGCATCCACCTTCCCCAGTCATGTTCACATGATAA
GATGAAGAAAAAATAGGCGCCCTCAACTTGACGGC
GCCTATGATGGACAGCTCAACGTATTTTGACTTTT
GGTGGCAGTATTGAGCGATTGACGTTGTTAGTTTT
TAAGGATATTCGGAAAAGAGTGTGTATCGGGTAAG
TTAAAATAATATCAAATTAACACTTGCAACCGGTC
GCAATTGCGACCATAATGTATTCAACGGTTCGGAA
CTGGTTCGAATCGACTCAAGAAAGTGAGATACATA
(Vibrio cyclitrophicus)
SEQ ID NO. 39
TCTTGAAACCTGTTACATAATTCATAGTTTTGATT
AGTGTAACGGTAATCAAAACTCGTCACAAGATATA
CAAAACGGGTATTCGGAAAAGAGTGTGTATCGGAT
AAGTTAAAATAAATTCAAATTAACACTTGCAAATG
GTCGCAATTGCGACCATAATATCTTCAACGGTTCG
GAACTGGTTCGAATCGACTCAAGAAAGTGAGATAC
ATCA
(Pectobacterium phage ZF40)
SEQ ID NO. 40
AGCCTCACCTCCGGCGTTGCCGTGGCGCTGTGTGA
TTTACAGGAAATAAAAAGGCCACGAATGCGGCCTT
AGCGATTAAAAAATATGAAATGCCTTGCTTGTTCG
CGATTGCGAACATATAATTTATTCATCGGTTCGAG
(Oceanimonas smirnovii)
SEQ ID NO. 41
TGATGTGTCTCTCTTTAGAGGTGATTCGAGCCAAT
CTCGAATCTGGTTCCATCTTAGTTCGCAATTGCGA
ACAGTGCAAGAGATAAAGTAAAGAAAATACAAAAA
TCAGCCAATCTGCTTCCTGGGGGTTAACGGTGAAG
CGTGGGGGCGAGCTTT
(Brackiella oedipodis)
SEQ ID NO. 42
AATCCAAACGTATTAATTTGTTGTTAAAATCCAAT
AAAAAACATTACAAAAGTATTGACAATAAATATTG
CATAAGTAATAATCAAACCATAGAATCACTTCTTG
CTCTTTAACAATCAACTGAATAGTCAGTCAGTCAA
CTGATAGAAACCTACTCCCAATGGAATAGACTTAT
GGGGTTCAACGGTCGGGCAGCCCCCAAACAGAATA
GCCGTGCGTGGTGAAAGCGCAGAGCCGATTAATTT
CC
Aca3-associated acr upstream regions
from Neisseria meninkitides
(Nme NmSL13x2)
SEQ ID NO. 43
AATTGAATCCGCAATGGTGAAATATCGACAATGAA
CGACAACACGCAAACCCTTCCCCCGCGCCACCTGT
CCGTCGCCCCGATGCTCGACTGTATCTACTGAAAA
ATAATATATTGAAAAATAATATATAATATATTTTT
ATTATTCTTATGGTGCAAATAAAGCACATTGTGCA
TTGGAAATAAAAACGGCAAATTAATTACCTTTGTT
TTTAAAGGTTTATTAAATTGGCGGTTTTTTGTTTG
AAAAAATGCTTGTTATACATCATTTTTTGATGTAG
TATACACACATCGGCAGACAACAAGCCTGCCACCG
ACACCTTGACGGTTTCAAGGATAAACGAAAGGATT
TCAAAA
(Nme 22472)
SEQ ID NO. 44
AATTGAATCCGCAATGATGAAATATCGACAATGAA
CGACAATACACACACCCTTCCCCCGCGCCGCCTTT
CCGTCGCCCCGATGCTCGACTGTATCTACTGAAAA
ATAATATATTGAAAAATAATATATAATATATTTTT
ATTATTCTTATGGTGCAAATAAAGCACATTGTGCA
TTGGAAATAAAAACGGCAAATTAATTACCTTTGTT
TTTAAAGGTTTATTAAATTGGCGGTTTTTTGTTTG
AAAAAATGCTTGTGATACATCATTTTTTGATGTAT
CATACACACATCGGCAGACAACAAGCCTGCCGCCG
CCACCTTGACGGTTTCAAGGATAAACGAAAGGATT
TCAAA
(Nme M40030)
SEQ ID NO. 45
GATAATATCCCCCCGTCCGCAACCGTTCAAACGAC
TAAGGAGGCAAAATGGCATATCGGTTCAAAACAGG
CGTGATTGCCGGAATCCCGACTGTATCTACTGAAA
AATAATATATTGAAAAATAATATATAATATATTTT
TATTATTCTTATGGTGCAAATAAAGCACATTGTGC
ATTGGAAATAAAAACGGCAAATTAATTACCTTTGG
TTTCAAAGGTTTATTAAATTTGCCGTTTTTTGTTT
GAAAAAGTGCTTGTGATACATCATTTTTTGATGTA
GTATACACACATCGGCAGACAACAAGCCTGCCACC
GACACCTTGACGGCTTCAAGGATAAACGAAAGGAT
TTCAAA
(Nme 2842STDY5881035)
SEQ ID NO. 46
AATTGAATCCGCAATGGTGAAATATCGACAATGAA
CGACAATACACACACCCTTCCCCCGCGCCACCTGT
CCGTCGCTCCCATGCTCGACTGTATCTACTGAAAA
ATAATATATTGAAAAATAATATATAATATATTTTT
ATTATTCTTATGGTGCAAATAAAGCACATTGTGCA
TTGGAAATAAAAACGGCAAATTAATTACCTTTGTT
TTTAAAGGTTTATTAAATTGGCGGTTTTTTGTTTG
AAAAAATGCTTGTGATACATCATTTTTTGATGTAG
TATACACACATCGGCAGACAACAAGCCTGCCACCG
ACACCTTGACGGCTTCAAGGATAAACGAAAGGATT
TCAAA
(Nme NM80179)
SEQ ID NO. 47
AATTGAATCCGCAATGATGAAATATCGACAATGAA
CGACAATACACACACCCTTCCCCCGCGCCGCCTTT
CCGTCGCCCCGATGCTCGACTGTATCTACTGAAAA
ATAATATATTGAAAAATAATATATAATATATTTTT
ATTATTCTTATGGTGCAAATAAAGCACATTGTGCA
TTGGAAATAAAAACGGCAAATTAATTACCTTTGTT
TTCAAAGATTTATTAAATTTGCCGTTTTTTGTTTA
AAAAAGTGCTTGTGATACATCATTTAATGATGTAA
TATACACACATGGACAGACAACAAGCCTGCCACCG
ACACCTTGACGGATTCAAGGATAAACGAAAGGATT
TCAAAA
(Nme 28425TDY5881013)
SEQ ID NO. 48
ATCTAACCCTATCAGCAAACGGCAAATTAATTACC
TTTGGTTTCAAAGGTTTATTAAATTTGCCGTTTTT
TGTTTAAAAAAGTGCTTGTGATACATCATTTAATG
ATGTAATATACACACATGGACAGACAACAAGCCTG
CCACCGACACCTTGACGGATTCAAGGATAAACGAA
AGGATTTCAAA
(Nme WUE2121)
SEQ ID NO. 49
ATTTAATTCTATTAAATAAACGGCAAATTAATTAC
CTTTGGTTTCAAAGGTTTATTAAATTTGCCGTTTT
TTGTTTAAAAAAGTGCTTGTGATACATCATTTAAT
GATGTAATATACACACATGGACAGACAACAAGCCT
GCCACCGACACCTTGACGGATTCAAGGATAAACGA
AAGGATTTCAAA
AcrIIA1 sequences
(AcrIIA1_LmoJ0161)
SEQ ID NO: 50
MTIKLLDEFLKKHDLTRYQLSKLTGISQNTLKDQN
EKPLNKYTVSILRSLSLISGLSVSDVLFELEDIEK
NSDDLAGFKHLLDKYKLSFPAQEFELYCLIKEFES
ANIEVLPFTFNRFENEEHVNIKKDVCKALENAITV
LKEKKNELL*
(AcrIIA1_LM010)
SEQ ID NO: 51
MSIKLLDEFLKKHNKTRYQLSKLTGISQNTLNDYN
KKELNKYSVSFLRALSMCAGISTFDVFIFLAELEK
SYDDLAGFKYLLDKHKLSFPTQEFELYCLIKHFES
ANIEVLPFTFNRFENETHADIEKDVKKALNNAIAV
LEEKKRRTVIKTIDYYDYS*
(AcrIIA1_LmoCFSAN026587.)
SEQ ID NO: 52
MNILDEFLNEHQITRYRLSKITGISNQLLLQYTKK
TLEEYPVWLLRALAAATDQTIEEVLNKLEILETEK
HQLYGIRSFLEKYNCSFPQEEWMLYRALYLVEALN
MDLEEMKFDRFEKEEHANIEKDVQEAVSNAVSTID
MIRRKKLKGHFKN*
(AcrIIA1_LmoFRRB2887)
SEQ ID NO: 53
MKTNLLDTFLKRHGITRYRLSKLAGISQNTLKDYT
EKSLNKYTVSFLRSLSFVTGEDVTDVLLELAEIEN
GYDDLAGFKYLLDKYKLSFPALEFELYCIIKEFES
ANIEISPFTFNRFENETHVDIEKDVKKALQNAVTV
LEERKEELL*
(AcrIIA1_Lsee)
SEQ ID NO: 54
MKINLLDEFLKRHNITRYRLSKLAGISQNTLKDYT
EKSLNKYTVSFLRSLSFATGESVTDILLELAELEK
DYDDLAGFKYLLDKYKLAFPALEFELYCLIKEFES
ANIEISPFTFNRFESETHTDIEKDVKKALQNAVTV
LEERKEELL*
(AcrIIA1_Eriv)
SEQ ID NO: 55
MNKFIIHYLKIERKQTMNLLDKFLNKRNLTRQQLS
NISGYSTGRLFDYNNKELNKYPVALLRTLAKISSM
SLTDTLKELEEIEASYDSLLGFRKLLEQYELSFPD
LEFELYCTIKDLESLKVKVEPFTFNRFEEEGHNNI
ASDCRKAMENAISMLSEALENVRKGKAPFEDEEI*
(AcrIIA1_Lgel)
SEQ ID NO: 56
MKLDDYLKLNNTTRYEVAKISGIPETSFKSIRNRD
VNNLSGRFYRAIGLVLGKTGGQVYDEITADENTVF
NFLGKHHIHDKERVTELLDYMLYFKKHDIDVTNVS
FNRFENEIENGHILGDEDDVLQVIDNLIESFKTMK
ENVEAGNLPTPEKMD*
(orfB_LmoJ0161)
SEQ ID NO: 57
MNNHVIDLTNKKFGRLTVKEFVRSENGNALWNCFC
VCGNEKEVLAQHLKRGHVQSCGCLARDNGRKHADK
NLRSETAQKNALKRKLEVDAVDGTMKSALTRSLSA
RNKSGIKGVRWDEKRNKWEASITFQKKLHFLGRFE
KKDDAVKARRDAEDKYFKPILDKMN*
(orfD_LmoFSLJ1-208)
SEQ ID NO: 58
MKGFLKRYAQEKKGWSLYKLAKESGIQDTTLSFAN
SKSVHNLSALNIKLISEAVGETPGTVLDELTELEK
EMEMETTYWYNEGTGTLLTWKEYKAKIESEARDWL
EDLQEEEEELDDSDKTSLETLVQLSFENESDFVLS
DSEGNPIKEW*
(orfD_ϕP70)
SEQ ID NO: 59
MNELRSLEMSINAKDYATRLESGEGSLYIRFGDSE
DYPVHASTNSTIKETFIELFKNGWNGYEEDEQELA
EDMQEIAQELILEELTDIFEEYEFSTDEIDTDLFS
GFTFHVDMDNDEAVYLMDAINATKYFEARPSSWYA
LLEVSYCG*
(orfJn2_Efae)
SEQ ID NO: 60
MKGLLELSTIDLFLKKYGITRNKVATQNIKHKISN
NALAQANLRPVETYSVKLILGLSEAVNEAPEKVMA
QLLEIEKSQTNSESAQKKEAYQFGNIILEGILNTN
RSTHEIRLVQYLGKRTLFCTYVSGVGAMNWSVSDY
KEIAETLKIDDVDIRFRTSENDQFWDVSESYRY*

In embodiments where a polynucleotide is introduced that encodes an appropriate Aca, any suitable promoter can be used that will lead to a level of expression that is higher than the level in the absence of the construct. Any level of expression that is sufficient to bind to the acr promoter, and in particular an inverted repeat within the promoter, e.g., an IR2 repeat, and to decrease the level of transcription of the acr can be used. It will be appreciated that in some embodiments, particularly in self-targeting strains, there may already be a certain amount of endogenous Aca protein present in the cells, but at a level that is insufficient to abolish Acr expression, with the result that CRISPR-Cas activity is still inhibited in the cells. In such cells, the introduction of the Aca according to the present methods will lead to an increased level of Aca activity in the cells, resulting in a decrease in Acr expression and activation of CRISPR-Cas.

In some embodiments, the promoter will be a constitutive promoter, such as the native acr-aca promoter or a housekeeping gene in the targeted microbe, or an inducible promoter such as aTC, IPTG, or a promoter responsive to arabinose induction.

The Aca protein can be delivered in any of a number of ways to the targeted prokaryotic cells, including by transferring the protein itself and by transferring polynucleotides encoding the protein, wherein the protein is expressed within the cell.

In some embodiments, the Aca protein or Aca-encoding polynucleotide is introduced together with, or in conjunction with, the delivery of a guide RNA. In such embodiments, the guide RNA will direct endogenous or exogenous CRISPR-Cas to target the nucleic acid whose sequence matches that of the guide RNA and, depending on the CRISPR-Cas system used, will cleave, nick, edit, modulate the transcription of, label, or otherwise modify the targeted locus. Any guide RNA can be used in the present methods, with no limitations. In one embodiment, the guide RNA targets a multidrug resistance sequence in bacteria, such that the active CRISPR-Cas system in the presence of the introduced Aca protein directs the targeting and degradation of the sequence, thereby selectively killing cells bearing the sequence or the selective destruction of plasmids bearing the sequence.

In other embodiments, the guide RNA is used to specifically target particular cells, e.g., pathogenic cells, within a mixed population of cells in vivo. In such embodiments, the guide RNA can be used to direct the cleavage, for example, of pathogenic cells by targeting a nucleic acid sequence specific to the pathogenic cells.

Introduction of an Aca as described herein into a prokaryotic cell can be achieved by any method used to introduce protein or nuclei acids into a prokaryote. In some embodiments, the Aca polypeptide is delivered to the prokaryotic cell by a delivery vector (e.g., a bacteriophage) that delivers a polynucleotide encoding the Aca polypeptide.

In some embodiments, polynucleotides, e.g., encoding one or more Aca polypeptide or one or more CRISPR-Cas component, e.g., a guide RNA or Cas protein, are introduced into bacteria using phage, e.g., a phage delivery vector comprised of ssDNA or dsDNA that delivers DNA cargo to target cells. Any phage capable of introducing a polynucleotide into the target cell can be used. The phage could be, e.g., a tailed phage or a filamentous phage, that carries an entirely designed genome or that has heterologous genes introduced into an otherwise natural genome.

In other embodiments, polynucleotides, e.g., encoding one or more Aca polypeptide or one or more CRISPR-Cas component, e.g., a guide RNA or Cas protein, are introduced into bacteria using bacterial conjugation. In some embodiments, polynucleotides are introduced into target prokaryotes using E. coli as a conjugative donor strain, e.g., using mobilizable plasmids that transfer their genetic material, e.g., polynucleotides encoding one or more Aca polypeptide or one or more CRISPR-Cas component.

An Aca polypeptide as described herein can be introduced into any cell that contains, expresses, is expected to express, or potentially expresses, an Acr protein. Exemplary prokaryotic cells can include, but are not limited to, those used for biotechnological purposes, the production of desired metabolites, E. coli and human pathogens. Examples of such prokaryotic cells can include, for example, Escherichia coli, Pseudomonas sp., Corynebacterium sp., Bacillus subtitis, Streptococcus pneumonia, Pseudomonas aeruginosa, Staphylococcus aureus, Campylobacter jejuni, Francisella novicida, Corynebacterium diphtheria, Enterococcus sp., Listeria monocytogenes, Mycoplasma gallisepticum, Streptococcus sp., or Treponema denticola.

In any of the embodiments described herein, one or more Aca polypeptide(s) can be introduced into a cell to allow for binding to one or more Acr promoter(s) and inhibition of Acr expression, together with a CRISPR-Cas polynucleotide. These different components (e.g., the different Aca polypeptides, or polynucleotides encoding the polypeptides, and the different CRISPR-Cas components) can be introduced together, e.g., within the same plasmid or phage, or in series. In some embodiments, an Aca polypeptide as described herein can be introduced (e.g., administered) to an animal (e.g., a human), for example an animal suffering from a bacterial infection, wherein the Aca polypeptide is directed to infectious bacteria within the animal

In some such embodiments, the Aca polypeptides or a polynucleotide encoding the Aca polypeptide, in administered as a pharmaceutical composition. In some embodiments, the composition comprises a delivery system such as a liposome, nanoparticle or other delivery vehicle as described herein or otherwise known, comprising the Aca polypeptides or a polynucleotide encoding the Aca polypeptide, to target bacteria, intracellular or otherwise, within the subject. The compositions can be administered directly to a mammal (e.g., human) using any route known in the art, including e.g., by injection (e.g., intravenous, intraperitoneal, subcutaneous, intramuscular, or intrademal), inhalation, transdermal application, rectal administration, or oral administration.

In some embodiments, e.g., when the bacteria to be targeted are present within mammalian host cells, two-fold delivery systems can be used, e.g., with an initial system to target the particular mammalian cell type that harbor the infectious bacteria so as to deliver the phage or other system for delivering the Aca polynucleotide, and then a second system to deliver the phage to the intracellular bacteria. See, e.g., Greene (2018).

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

### EXAMPLES

The present invention will be described in greater detail by way of specific examples. The following examples are offered for illustrative purposes only, and are not intended to limit the invention in any manner Those of skill in the art will readily recognize a variety of noncritical parameters which can be changed or modified to yield essentially the same results.

### Example 1. Anti-CRISPR Associated Proteins are Crucial Repressors of Anti-CRISPR Transcription

### Introduction

Phages express anti-CRISPR proteins to inhibit CRISPR-Cas systems that would otherwise destroy their genomes. Most anti-CRISPR (acr) genes are located adjacent to anti-CRISPR associated (aca) genes, which encode proteins with a helix-turn-helix DNA-binding motif. The conservation of aca genes has served as a signpost for the identification of acr genes, yet the function of the proteins encoded by these genes has not been investigated. Here, we reveal that an acr associated promoter drives high levels of acr transcription immediately after phage DNA injection, and that Aca proteins subsequently repress this transcription. In the absence of Aca activity, this strong transcription is lethal to a phage. Our results demonstrate how sufficient levels of anti-CRISPR protein accumulate early in the infection process to inhibit existing CRISPR-Cas complexes in the host cell. They also imply that the conserved role of Aca proteins is to mitigate the deleterious effects of strong constitutive transcription from acr promoters.

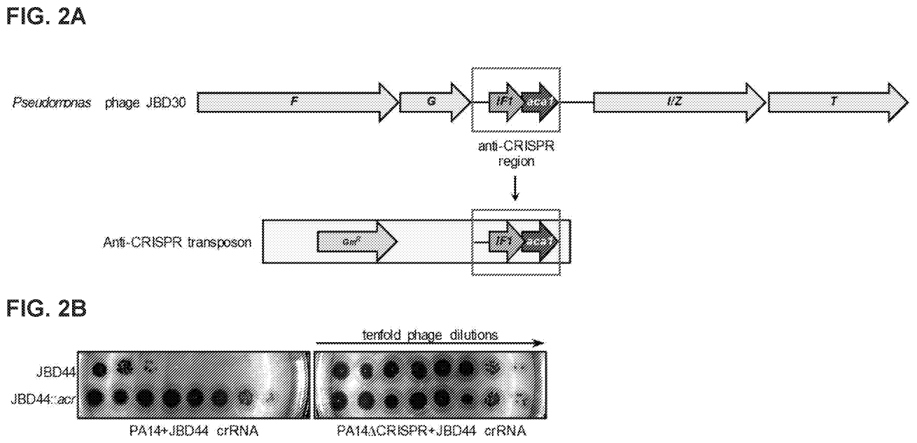

The goal of this work was to define the role of aca genes in anti-CRISPR biology. We investigated aca gene function using Pseudomonas aeruginosa phage JBD30 as our primary model system (FIG. 2A). This phage was among the first set of phages shown to use an anti-CRISPR gene for survival in the presence of CRISPR-Cas (Bondy-Denomy et al., 2013). The anti-CRISPR operon of JBD30 and other closely related phages is located between operons encoding phage structural proteins. In JBD30, a single anti-CRISPR (acr) gene, acrIF1, is followed directly by an aca gene, known as aca1 in these phages. Aca1 is conserved (>50% identity) among diverse anti-CRISPR encoding phages and prophages in Pseudomonas species (Pawluk et al., 2016b). Since Aca1 possesses a HTH DNA-binding motif, we speculated that it might be involved in anti-CRISPR gene expression. Consequently, we considered possible mechanisms by which anti-CRISPR proteins deploy during phage infection to prevent genome destruction by pre-formed CRISPR-Cas complexes. We found that anti-CRISPR transcription occurs at high level early in the phage infection process, and that Aca1 represses this transcription. Remarkably, the repressor activity of Aca1 is essential for phage survival irrespective of CRISPR-Cas. We also showed that other Aca protein families act as repressors of anti-CRISPR transcription. This crucial function of Aca has likely contributed to its ubiquity in anti-CRISPR operons.

### Results

Anti-CRISPR protein is not packaged into phage particles. To begin addressing how anti-CRISPRs are deployed during the phage infection process, we looked at whether these proteins were packaged into phage particles. Anti-CRISPR proteins could protect the phage genome immediately after injection if injected from the phage particle into the cell alongside the phage DNA. Packaging of phage-encoded inhibitors of bacterial defense systems has been documented previously. For example, E. coli phages T4 and P1 both incorporate protein inhibitors of restriction endonucleases into their capsids and deliver them along with their genomes to protect against host defenses (Bair et al., 2007; lida et al., 1987; Piya et al., 2017). To assay for the presence of anti-CRISPR protein in particles of the AcrIF1-encoding phage JBD30, we performed mass spectrometry on purified phage particles. While we detected all expected virion proteins with high confidence, the anti-CRISPR protein was not detected (Table 2). The small size of AcrIF1 (78 amino acids) does make it less likely to be detected by mass spectrometry. However, we were able to detect with 100% confidence the 138 amino acid head-tail connector protein and the 157 amino acid tail terminator protein, which are likely present in 12 (Cardarelli et al., 2010) and 6 (Pell et al., 2009) copies, respectively, per phage particle. A similar mass spectrometry experiment performed on phage JBD88a, which encodes two anti-CRISPR proteins, also failed to detect these in purified phage particles (Harvey et al., 2018). Considering that the anti-restriction proteins of phages P1 and T4 are present at greater than 40 copies per phage particle (Bair et al., 2007; Piya et al., 2017), we anticipated that we would have detected anti-CRISPR protein in the phage particles if they were packaged.

For anti-CRISPR proteins to be packaged into phage particles, recognition between a virion protein and the anti-CRISPR would be required. Thus, an anti-CRISPR from one phage would not be expected to function within the context of a completely different phage. To test this idea, we incorporated the anti-CRISPR region of phage JBD30 (FIG. 2A) into random locations in the genome of the unrelated D3-like P. aeruginosa phage JBD44 using transposon mutagenesis. Even though the virion proteins of JBD44 are completely unrelated to those of JBD30, the acrIF1 region inserted into JBD44 was still able to confer resistance against the type I-F CRISPR-Cas system of P. aeruginosa strain UCBPP-PA14 (PA14). The plaque-forming ability of wild-type JBD44 was robustly inhibited when targeted by the PA14 CRISPR-Cas system, while JBD44 phages carrying the anti-CRISPR region (JBD44::acr) were protected from CRISPR-Cas mediated inhibition (FIG. 2B). Additionally, the level of plaquing by JBD44::acr phages was the same regardless of the presence or absence of a CRISPR-Cas system, suggesting that these phages exhibit full anti-CRISPR activity. These results demonstrate that AcrIF1 retains full functionality in the genomic context of an entirely different phage. This implies that interaction between the anti-CRISPR and other phage components (including the virion proteins) is not required.

The acrIF1 gene is robustly transcribed from its own promoter at the onset of phage infection. The distinct transcription profile of the acrIF1 gene implied that it possessed its own promoter. A DNA sequence alignment of the region upstream of diverse acr genes from phages related to JBD30 revealed a conserved predicted promoter (FIG. 3B). This region from phage JBD30 was cloned upstream of a promoterless lacZ reporter gene carried on a plasmid. The presence of the putative acrIF1 promoter increased β-galactosidase activity by approximately 15-fold when compared to the control lacking a promoter, demonstrating that this DNA sequence can direct robust transcription in P. aeruginosa (FIG. 3C). To confirm that this promoter was responsible for anti-CRISPR gene expression during phage infection, we created a JBD30 mutant phage (JBD30ΔPacr) lacking this region. In a phage plaquing assay, the JBD30ΔPacr mutant phage replicated robustly on PA14 lacking a functional CRISPR-Cas system (PA14ΔCRISPR), but in the presence of CRISPR-Cas immunity phage replication was equivalent to that of a JBD30 mutant bearing a frameshift mutation in acrIF1 (acr_{fs}) (FIG. 3D). These data imply that the identified promoter drives acrIF1 transcription during infection.

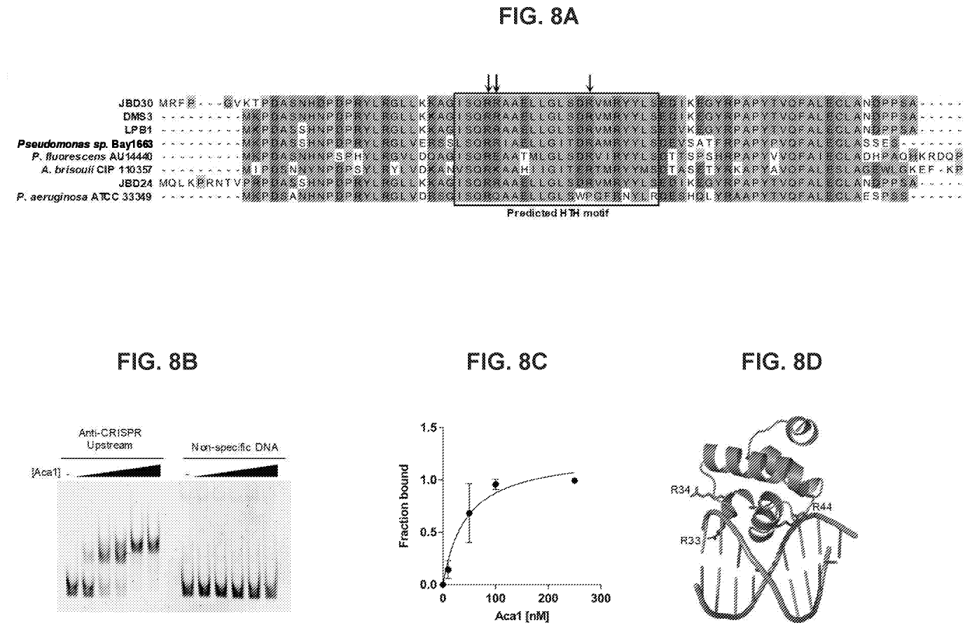

Aca1 acts on the acr promoter. Aca1 proteins are bioinformatically predicted to contain a helix-turn-helix (HTH) DNA-binding motif (FIG. 8A). HTH-containing proteins are generally dimeric and bind to inverted repeat sequences. We identified two such sites with very similar sequences which we refer to as IR1 and IR2, flanking the −35 region of the acrIF1 promoter (FIG. 3B). To determine whether Aca1 could bind to the anti-CRISPR promoter region, purified Aca1 was mixed with a 110 bp dsDNA fragment containing the acr promoter and an electrophoretic mobility shift assay (EMSA) was performed. Incubation of the promoter-containing fragment with Aca1 resulted in a concentration-dependent shift in the mobility of the fragment, which was not observed with a non-specific DNA sequence (FIG. 8B). At higher Aca1 concentrations, a second shifted band was observed, consistent with the presence of two Aca1 binding sites within this fragment. The dissociation constant (K_{d}) of this interaction was approximately 50 nM (FIG. 8C). A 53 bp fragment encompassing only IR1 and IR2 of the acr promoter also bound to Aca1 and displayed two shifted bands by EMSA (FIG. 3E). Fragments bearing mutations in either IR1 or IR2 still bound to Aca1, but only a single shifted band was observed, while no shift was observed with a fragment bearing mutations in both sites. These results demonstrated that Aca1 binds the acrIF1 promoter at both the IR1 and IR2 sites.

Given the binding of Aca1 to the acrIF1 promoter, we speculated that this might contribute to the strong transcription of this gene early in infection. To determine whether Aca1 binding to the acrIF1 promoter modulates its transcriptional activity, we measured the activity of this promoter in the presence of Aca1 using the lacZ reporter assay described above. Contrary to our expectation, the presence of Aca1 in this assay led to a five-fold reduction in β-galactosidase reporter activity (FIG. 3F). This repressive activity of Aca1 depended on the presence of an intact IR2 site, suggesting that this site is active in vivo. By contrast, the IR1 site was not required for repression despite being bound by Aca1 in vitro. The in vivo function of the Aca1 binding sites in the acrIF1 promoter were assessed by crossing the inverted repeat mutations into phage JBD30 through in vivo recombination (Bondy-Denomy et al., 2013). Despite the marked effect of the IR1 and IR2 mutations on Aca1 DNA binding in vitro, introduction of these mutations into the phage genome caused no significant decrease in the viability of the mutant phages on either wild-type PA14 or PA14ΔCRISPR (FIG. 3G).

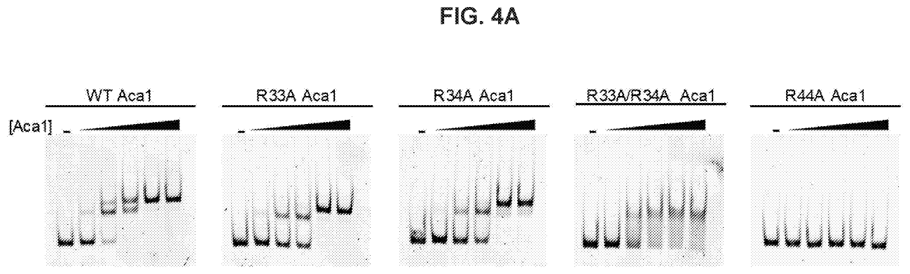

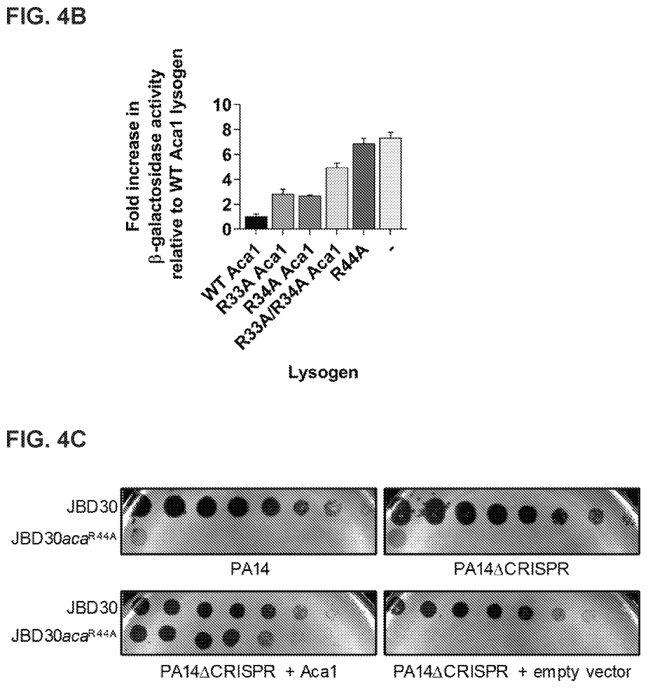

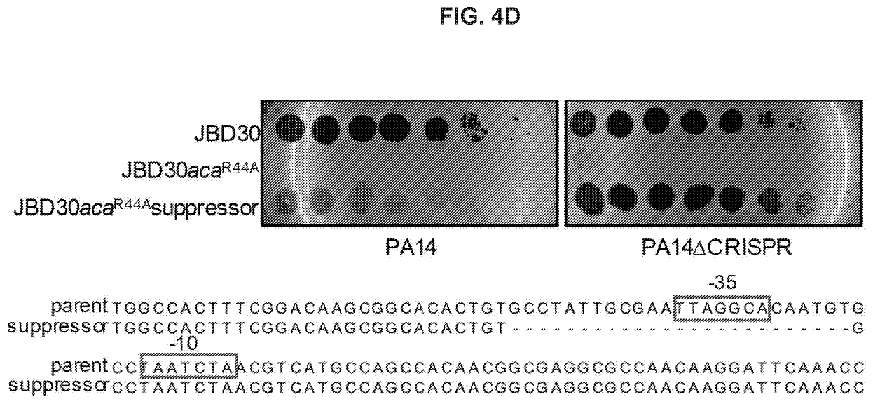

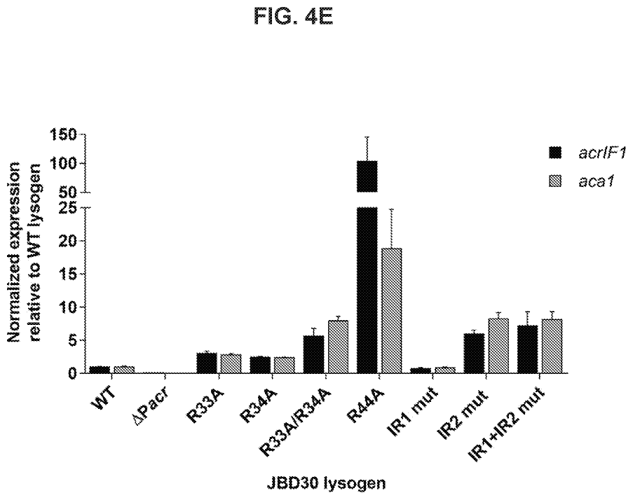

Aca1 repressor activity is required for phage viability. To further investigate the role of the Aca1 DNA-binding activity, we introduced amino acid substitutions within the putative HTH region of Aca1 that were expected to reduce DNA-binding (FIG. 8D). Substitutions with Ala at Arg33 or Arg34 and an Arg33/Arg34 double mutant each partially reduced the DNA-binding activity of Aca1 in vitro, while substituting Arg44, which is predicted to be in the major groove recognition helix, completely abolished Aca1 DNA-binding (FIG. 4A). The DNA-binding activity of these mutants was also measured using the lacZ reporter assay. Consistent with the in vitro data, the R44A mutant displayed very little repressor activity on the acrIF1 promoter (FIG. 4B). The activity of the R33A/R34A double mutant was intermediate between the R44A mutant and the R33A and R34A single mutants, corroborating the in vitro changes in DNA-binding activity observed for these mutants.

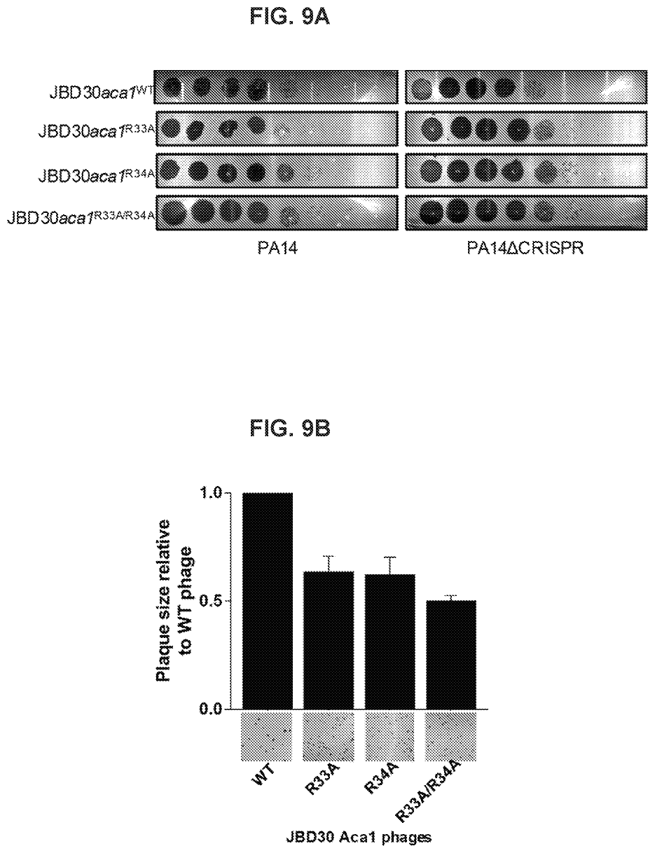

The Aca1 DNA-binding mutants were subsequently crossed into phage JBD30. Unexpectedly, we were able to isolate phages carrying the mutations affecting Arg33 and Arg34, but not the mutation affecting Arg44. The R44A mutant phage could only be obtained by plating on cells expressing wild-type Aca1 from a plasmid, suggesting that the Aca1 DNA-binding activity is essential for phage viability. Using high titer lysates of JBD30aca1^{R44A} produced in the presence of Aca1, we discovered that this phage was unable to replicate (titer reduced >10⁶-fold) on wild-type PA14 or PA14ΔCRISPR (FIG. 4C). By contrast, the mutant phages encoding Aca1 substitutions at the Arg33 or Arg34 positions formed plaques at levels approaching that of the wild-type phage (FIG. 9A). These data demonstrate that intermediate reductions of Aca1 DNA-binding activity have little effect on phage viability, but that a complete loss of Aca1 DNA-binding activity is lethal.

Although the JBD30aca1^{R44A} phage replicated very poorly on the PA14ΔCRISPR strain, plating high concentrations of this phage did lead to the appearance of revertant plaques at a low frequency (<1×10⁻⁶). Sequencing the anti-CRISPR regions of several of these revertants revealed that they still carried the aca1^{R44A} mutation. Most also displayed a 25 bp deletion encompassing the −35 region of the acrIF1 promoter (FIG. 4D). These revertants were able to plate to the same level as wild-type JBD30 on the PA14ΔCRISPR strain, but showed a marked reduction in titer on wild-type PA14. This is what we would expect to see if the acr promoter were impaired as demonstrated in FIG. 3D. This result implies that the inviability of the aca1^{R44A} mutant phage arises from the high transcription level at the acr promoter. As a result, deletion of a critical portion of this promoter is able to restore viability.

To verify the transcriptional effects of mutations in the JBD30 acr promoter and aca1 gene, we performed RT-qPCR. These assays were carried out on strains that had been lysogenized with mutant phages (i.e., the phage genomes were integrated into the PA14ΔCRISPR genome to form a prophage). In the lysogenic state, acr expression must persist to prevent the host CRISPR-Cas system from targeting the prophage, which would be lethal. Performing assays in the lysogenic state allowed us to assess transcription levels at a steady state as opposed to the dynamic situation existing during phage infection. Both the acrIF1 and aca1 genes were transcribed from the JBD30 prophage (FIG. 4E). The transcription of both genes was more than 20-fold lower in the phage mutant lacking the acr promoter, confirming the key role of this promoter in transcribing both of these genes. By contrast, the JBD30aca1^{R44A} mutant displayed vastly increased levels of acrIF1 and aca1 transcription (100-fold and 20-fold increases, respectively). Prophages expressing Aca1 mutants that bound DNA at somewhat reduced levels in vitro (i.e., substitutions at Arg33 and Arg34, FIG. 4A) also displayed increased transcription of the acrIF1 and aca1 genes but not nearly to the same degree as the JBD30aca1^{R44A} mutant. Mutations in IR2 that that caused loss of repression in the lacZ reporter assay (FIG. 3F) also resulted in increased acrIF1 and aca transcription. However, this increase was similar to that of the JBD30aca^{R33A/R34A} mutant, which was 15-fold lower than the JBD30aca^{R44A} mutant. The reduced transcription level of the IR2 mutants compared to the aca1^{R44A} mutant may be due to the base substitutions in IR2 (i.e., these changes may affect promoter strength) and/or there may be residual binding not detected in EMSA of Aca1 to the mutated operator that leads to some degree of repression.

The uniquely high transcription level from the acr promoter resulting from the aca1^{R44A} mutant provides a likely explanation for the inviability of the JBD30aca^{R44A} mutant phage while the phages bearing mutations in aca1 or the acr promoter retained their replicative ability. It is notable that examination of plaque sizes resulting from infection by wild-type and JBD30 phages bearing other aca1 mutations showed that the Arg33 and Arg34 substitutions measurably decreased phage replication (FIG. 9B). Thus, the more modest increases in acr promoter activity seen for these mutants still influenced phage viability. All of these data support the conclusion that the key function of Aca1 is not in activation, but in repression of acr transcription.

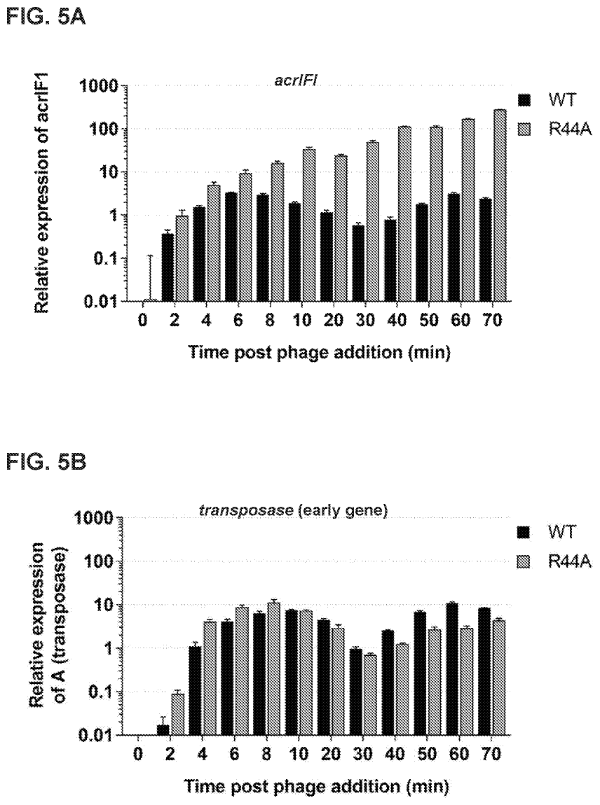

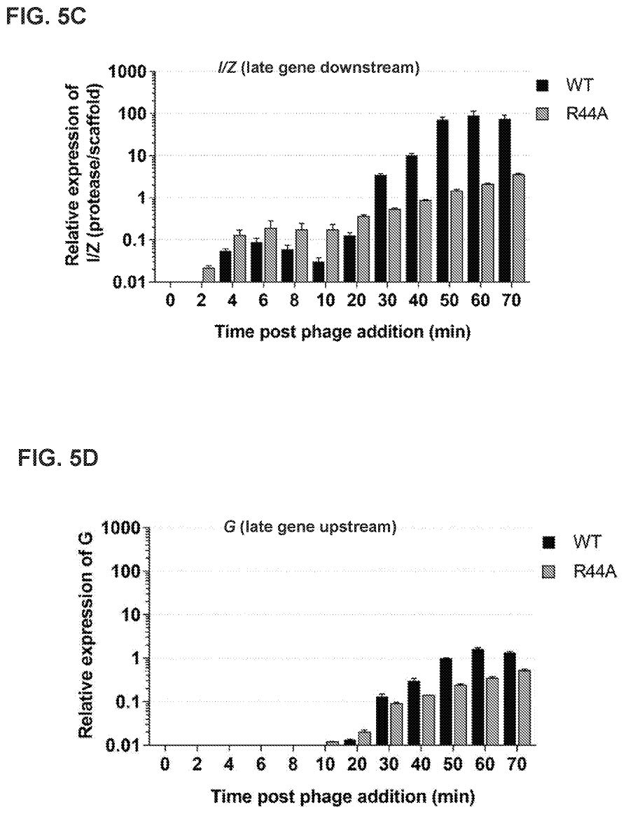

acr promoter activity is strong during early infection independent of Aca1. To directly address the role of Aca1 early in the phage infection process, we infected cells with wild-type JBD30 or the JBD30aca1^{R44A} mutant, and measured transcript accumulation using RT-qPCR as described above. Very early in infection, acrIF1 transcripts accumulated to high levels in both wild-type and mutant phage (FIG. 5A), clearly demonstrating that Aca1 is not required for rapid expression from the acr promoter. At later time points, acrIF1 transcripts accumulated to much higher levels in the JBD30aca1^{R44A} mutant, consistent with the repressor activity of Aca1. The transcription of the transposase gene varied relatively little between the wild-type and mutant phages (FIG. 5B). It should be noted that transcription of both the acrIF1 and transposase genes was observed earlier in these experiments than in those shown in FIG. 2A due to the use of a higher multiplicity of infection to improve the limit of detection in this assay. These results clearly demonstrate that Aca1 is not required for the early activation of acr transcription. Consequently, the importance of Aca1 must be derived from its ability to repress the acr promoter.

Loss of Aca1 repressor activity alters the transcription of downstream genes. In light of the results above, we postulated that the loss of viability observed for the JBD30aca1^{R44A} mutant was brought about by uncontrolled transcription from the very strong acr promoter. With the expectation that this inappropriate acr transcription might perturb the transcription of downstream genes, we measured the transcript levels of the phage protease/scaffold (I/Z) gene (FIG. 2A), which lies immediately downstream of the anti-CRISPR locus. Strikingly, I/Z gene transcript levels in the JBD30aca^{R44A} mutant phage were dramatically decreased relative to wild-type phage, reaching nearly a 100-fold difference at the later time points (FIG. 5C). By contrast, the G gene, which lies immediately upstream of the anti-CRISPR locus, displayed less than 10-fold differences in transcript levels between the wild-type and mutant phages (FIG. 5D).

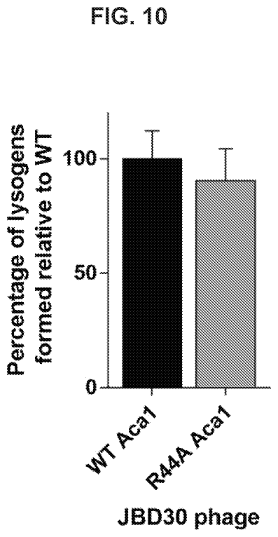

Based on genomic comparison with E. coli phage Mu, the I/Z gene is situated at the beginning of an operon that contains genes required for capsid morphogenesis (Hertveldt and Lavigne, 2008). The observed decrease in I/Z transcript level likely extends to other essential genes within this operon; thus, the JBD30aca^{R44A} mutant phage would lack sufficient levels of these morphogenetic proteins required for particle formation. This explains the observed loss of phage viability regardless of the CRISPR-Cas status of the host. Defects in virion morphogenesis could also lead to the small plaque phenotype observed in the partially incapacitated Aca1 mutants. In further experiments, we determined that the JBD30aca1^{R44A} phage forms lysogens with the same frequency as the wild-type phage (FIG. 10). Since lysogen formation does not require particle formation, this finding is consistent with the hypothesis that Aca1 debilitation causes a defect in phage morphogenesis.

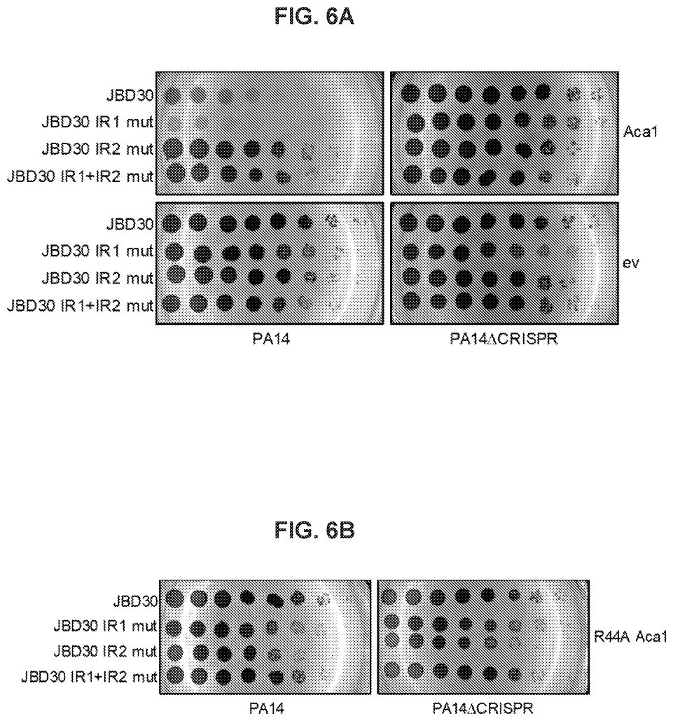

Aca1 can act as an “anti-anti-CRISPR”. Since Aca1 is a repressor of the anti-CRISPR promoter, we postulated that excessive Aca1 expression might inhibit the replication of phages requiring anti-CRISPR activity for viability in the presence of CRISPR-Cas. To test this, we plated phage JBD30 on wild-type PA14 cells in which Aca1 was expressed from a plasmid. We found that phage replication was inhibited by more than 100-fold in the presence of plasmid-expressed Aca1 as compared to cells carrying an empty vector (FIG. 6A). This loss of phage replication was CRISPR-Cas dependent, as plasmid-expressed Aca1 had no effect on phage replication in the PA14ΔCRISPR strain, indicating that the impairment of phage replication results from a decrease in anti-CRISPR expression. Importantly, phages bearing mutations in IR2, which is the binding site required for Aca1-mediated repression of the acr promoter, were able to replicate in the presence of excess Aca1. On the other hand, a phage mutated in the IR1 site, which binds Aca1 but does not mediate repression, replicated even more poorly than wild-type in the presence of Aca1. This confirms that binding of IR2 by Aca1 is required for repression of acr transcription in vivo, and indicates that IR1 titrates Aca1 away from IR2 and thereby lessens the repressive effect of Aca1. The DNA-binding activity of Aca1 is necessary to reduce JBD30 replication, as the overexpression of the Aca1R44A mutant has no impact on JBD30 replication in the presence of CRISPR-Cas (FIG. 6B).

Overall, the inhibitory effect of Aca1 on acr-dependent phage replication further bolsters our conclusion that Aca1 is a repressor of the acr promoter. This observation also raises the intriguing possibility that expression of Aca1 could be co-opted by bacteria as an “anti-anti-CRISPR” mechanism for protection against phages or other mobile genetic elements carrying anti-CRISPR genes.

Members of other Aca families are also repressors of anti-CRISPR promoters.

Genes encoding active anti-CRISPR proteins have been found in association with genes encoding HTH motif-containing proteins that are completely distinct in sequence from Aca1. For example, aca2 has been found in association with five different families of anti-CRISPR genes in diverse species of Proteobacteria (Pawluk et al., 2016a; Pawluk et al., 2016b). Genes encoding homologs of Aca3, another distinctive HTH-containing protein, have been identified in association with three different type II-C anti-CRISPR genes (Pawluk et al., 2016a). To investigate the generality of Aca function, we determined whether representative members of Aca2 and Aca3 families also function as repressors of anti-CRISPR transcription.

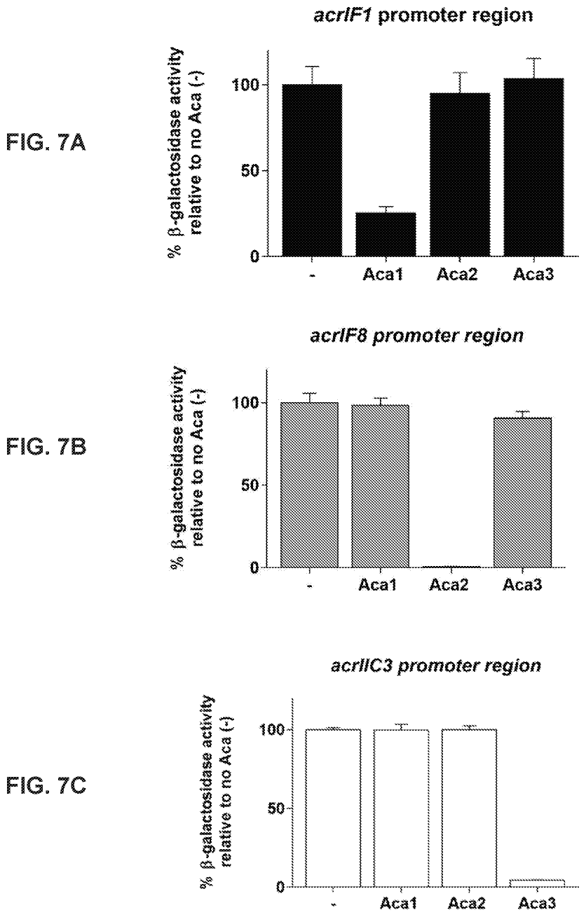

By aligning the intergenic regions found immediately upstream of anti-CRISPR genes associated with aca2, we detected a conserved inverted repeat sequence that could act as a binding site for Aca2 proteins (FIG. 11B). The same alignment approach also revealed an inverted repeat sequence that could act as a binding site for Aca3 (FIG. 11D). To investigate the functions of Aca2 and Aca3, the acrlaca regions from Pectobacterium phage ZF40 and from N. meningitidis strain 2842STDY5881035 were investigated as representatives of the Aca2 and Aca3 families, respectively. The putative promoter regions of the anti-CRISPR genes in these two operons (FIGS. 11B and 11D) were cloned upstream of a promoterless lacZ reporter gene carried on a plasmid. When assayed in E. coli, both regions mediated robust transcription of the reporter as detected by measuring β-galactosidase activity in cell extracts (FIG. 7). Co-expression of Aca2_{ZF40} and Aca3_{Nme} with their putative cognate promoters resulted in 100-fold and 20-fold reductions in β-galactosidase activity, respectively. Co-expression of aca2 with the aca3 operon promoter construct or vice versa did not exhibit any repression, demonstrating that the repressor activities of Aca2 and Aca3 are specific to their associated promoters. Overall these data show that, similar to Aca1, both Aca2 and Aca3 are repressors of anti-CRISPR transcription.

### Discussion

To date more than 40 families of anti-CRISPRs have been identified, inhibiting seven types of CRISPR-Cas systems. Each of these anti-CRISPR families is completely distinct in amino acid sequence from one another and bear no similarity to other known protein families Despite this diversity, genes encoding most anti-CRISPR families are found adjacent to genes encoding a predicted HTH-containing protein, or genes encoding an anti-CRISPR containing a HTH domain (AcrIIA1 and AcrIIA6) (Bondy-Denomy et al., 2013; He et al., 2018; Hynes et al., 2018; Hynes et al., 2017; Ka et al., 2018; Marino et al., 2018; Pawluk et al., 2016a; Pawluk et al., 2016b; Rauch et al., 2017). The ubiquity of this association between HTH proteins and anti-CRISPRs implies that these HTH proteins are carrying out a critical function. Here we have shown that Aca1, a HTH protein family linked with 15 families of anti-CRISPRs, is a repressor of anti-CRISPR transcription and is essential for phage particle production. In addition, we have explained the general necessity for modulation of anti-CRISPR transcription by an associated repressor. We found no evidence that AcrIF1 is incorporated into phage particles and injected into host cells along with phage DNA, and we would expect that this is also the case for other anti-CRISPRs. Thus, phage survival in the face of pre-formed CRISPR-Cas complexes in the host cell is dependent upon rapid high-level transcription of the anti-CRISPR gene from a powerful promoter. However, the placement of such strong constitutive promoters within the context of a gene-dense, intricately regulated phage genome is likely to result in the dysregulation of critical genes and a decrease in fitness. The inclusion of repressors within anti-CRISPR operons to attenuate transcription once sufficient anti-CRISPR protein has accumulated solves this problem. We surmise that the presence of aca genes within anti-CRISPR operons has been vital for the spread of these operons by horizontal gene transfer, allowing them to incorporate at diverse positions within phage genomes without a resulting decrease in phage viability.

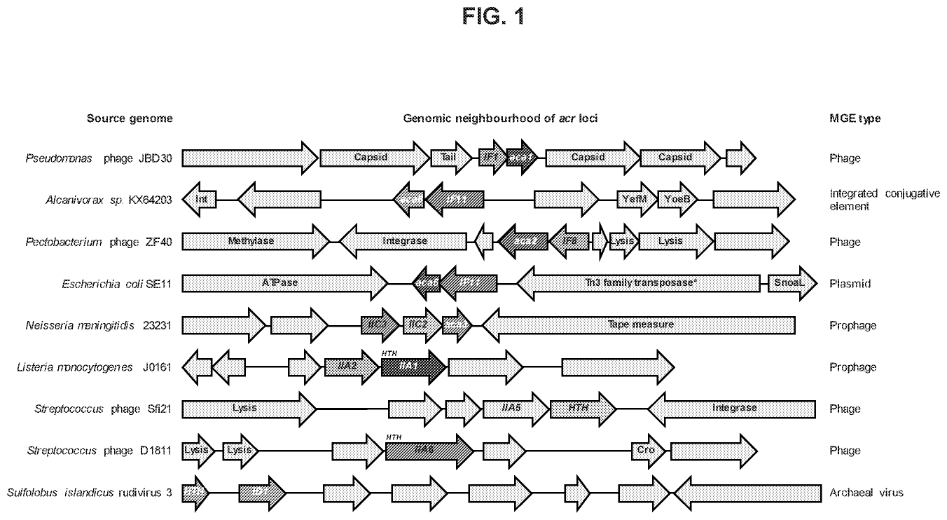

One question with respect to anti-CRISPR operons is how rapid high-level expression of anti-CRISPR proteins can be achieved when a repressor of the operon is produced simultaneously. Since Aca proteins are not present when phage DNA is first injected, initial transcription of anti-CRISPR operons is not impeded. In most anti-CRISPR operons the acr genes precede the aca gene and are thus translated first, allowing anti-CRISPR proteins to accumulate earlier. In addition, in JBD30 and related phages, the predicted strength of the aca1 ribosome binding site is at least 10-fold weaker than the acr site (Espah Borujeni et al., 2014; Salis et al., 2009; Seo et al., 2013), which would result in a slower accumulation of Aca1 protein. The same phenomenon was observed in the aca2- and aca3-controlled operons described above (FIG. 1). The presence of two binding sites for Aca1 in the acr promoter, only one of which mediates repression, may also serve to delay the repressive activity of Aca1. Evidence for this is seen in FIG. 6, where the replication of a phage lacking IR1 is inhibited to a greater extent than wild-type by plasmid-based expression of Aca1, presumably because Aca1 is normally titrated away from the IR2 site by binding to IR1. Other mechanisms to fine-tune the balance of anti-CRISPR and Aca protein levels, such as differential protein and/or mRNA stability, may also play a role in some cases. It is also important to consider that Aca proteins cannot be extremely strong repressors, as some level of anti-CRISPR transcription is required for the survival of temperate phages when they form prophages, which could be targeted by host CRISPR-Cas systems in the absence of anti-CRISPRs.

In the case of phage JBD30, we found that phage replication was abrogated in the absence of Aca1 function. This loss of viability appeared to be the result of a large decrease in the transcription of essential downstream genes (FIG. 5C). This gene misregulation is likely caused by readthrough transcription from the strong anti-CRISPR promoter. The genome organization and replication mechanism of JBD30 resembles that of the E. coli phage Mu (Hertveldt and Lavigne, 2008; Wang et al., 2004). In phage Mu, late gene expression is dependent on the C protein, a phage-encoded transcriptional activator (Margolin et al., 1989). JBD30 and other Pseudomonas Mu-like phages have a C protein homolog, and expression of the protease/scaffold, major head, and other essential genes is likely dependent on binding of this protein to a promoter region downstream of the anti-CRISPR operon. Thus, readthrough from the acr promoter may prevent the C protein from binding to key regulatory elements of the downstream operon, leading to reduced transcription. This possible explanation for the necessity of Aca1 in JBD30-like phages obviously would not apply to the different genomic locations of diverse anti-CRISPR operons. However, we expect that anti-CRISPR associated promoters would cause reduced viability when placed at many genomic locations in mobile DNA elements if these promoters were unregulated. These reductions in viability could be due to various mechanisms of gene misregulation. Consistent with this idea, highly expressed genes have generally been found to have a lower likelihood of horizontal transfer because of their greater potential to disrupt recipient physiology (Park and Zhang, 2012; Sorek et al., 2007).

It was recently shown that anti-CRISPR-expressing phages like JBD30 cooperate to inhibit the CRISPR-Cas system. Initial phage infections may not result in successful phage replication, but anti-CRISPR protein accumulating from infections aborted by CRISPR-Cas activity leads to “immunosuppression” that aids in subsequent phage infections (Borges et al., 2018; Landsberger et al., 2018). Through demonstrating that anti-CRISPR genes are expressed quickly after infection, we provide an explanation for how anti-CRISPR protein can accumulate even when phage genomes are ultimately destroyed by the CRISPR system. In the anti-CRISPR-expressing archaeal virus, SIRV2, the acrID1 gene was also transcribed at high levels early in infection, supporting the generalizability of this mechanism of anti-CRISPR action (Quax et al., 2013).

This work has answered two outstanding questions pertaining to the in vivo mechanism of anti-CRISPR activity. First, we demonstrate that acr genes are transcribed at high levels immediately after phage infection, illustrating how anti-CRISPRs are able to outpace CRISPR-Cas mediated destruction of the phage genome. Second, we establish a role for the highly conserved Aca proteins in diverse anti-CRISPR operons. This insight into anti-CRISPR operon function provides an explanation for their ability to integrate into different genomic locations across diverse mobile genetic elements. In addition, our work shows that Aca proteins have the potential to broadly inhibit anti-CRISPR expression, effectively acting as anti-anti-CRISPRs, which could have applications in CRISPR-based antibacterial technologies (Greene, 2018; Pursey et al., 2018).

TABLE 1
Key Resources

| | | |
|---|---|---|
| REAGENT or RESOURCE | SOURCE | IDENTIFIER |

Bacterial and Virus Strains

| | | |
|---|---|---|
| Pseudomonas aeruginosa strain | A. Davidson Lab | Refseq: NC_008463.1 |
| UCBPP-PA14 | | |
| Pseudomonas aeruginosa strain | G. O'Toole Lab | N/A |
| UCBPP-PA14 Δcas (PA14ΔCRISPR) | | |
| Pseudomonas phage JBD30 | A. Davidson Lab | Refseq: NC_020198.1 |
| Pseudomonas phage JBD30acr_{fs} | A. Davidson Lab | N/A |
| Pseudomonas phage JBD30ΔPacr | This study | N/A |
| Pseudomonas phage JBD30 IR1 mut | This study | N/A |
| Pseudomonas phage JBD30 IR2 mut | This study | N/A |
| Pseudomonas phage JBD30 IR1 + IR2 mut | This study | N/A |
| Pseudomonas phage JBD30aca^{R33A} | This study | N/A |
| Pseudomonas phage JBD30aca^{R34A} | This study | N/A |
| Pseudomonas phage JBD30aca^{R33A/R34A} | This study | N/A |
| Pseudomonas phage JBD30aca^{R44A} | This study | N/A |
| Pseudomonas phage JBD44 | A. Davidson Lab | Refseq: NC_030929.1 |
| Pseudomonas phage JBD44::acr | This study | N/A |
| Escherichia coli DH5α | A. Davidson Lab | N/A |
| Escherichia coli BL21(DE3) | A. Davidson Lab | N/A |
| Escherichia coli SM10λpir | K. Maxwell Lab | N/A |

Chemicals, Peptides, and Recombinant Proteins

| | | |
|---|---|---|
| Acid phenohchloroform | Ambion | Cat #AM9722 |
| Ni-NTA agarose resin | Qiagen | Cat #30210 |
| SYBR Gold nucleic acid stain | Invitrogen | Cat #S11494 |
| Purified protein: Aca1 | This study | N/A |

Critical Commercial Assays

| | | |
|---|---|---|
| TURBO DNA-free kit | Ambion | Cat #AM1907 |
| Superscript IV VILO master mix | Invitrogen | Cat #11754050 |
| PowerUp SYBR green master mix | Applied | Cat #A25741 |
| | Biosystems | |
| In-Fusion HD cloning kit | Clonetech | Cat #638912 |

Phusion High-Fidelity DNA Polymerase Thermo Scientific Cat #F530S
Oligonucleotides

| | | |
|---|---|---|
| For cloning, RT-qPCR, and EMSA | Eurofins Genomics | See Table S4 |
| Recombinant DNA | | |
| PHERD30T (gentR) | A. Davidson Lab | GenBank: EU603326.1 |
| pHERD30T derivatives | This study | See Table S5 |
| pHERD20T (ampR) | A. Davidson Lab | GenBank: EU603324.1 |
| pHERD20T derivatives | This study | See Table S5 |
| pBTK30 | S. Lorry Lab | N/A |
| pBTK30 derivatives | This study | Sec Table S5 |
| pCM-Str | N. Nodwell Lab | N/A |
| pCM-Str derivatives | This study | Sec Table S5 |
| pQF50 | A. Davidson Lab | N/A |
| pQF50 derivatives | This study | See Table S5 |
| p15TV-L | Addgene | ID #26093 |
| p15TV-L derivatives | This study | See Table S5 |

Sequence-Based Reagents

| | | |
|---|---|---|
| crRNA targeting JBD44 | This study | N/A |
| GGTTCACTGCCGTATAGGCAGCTAA | | |
| GAAAAGTTCCTTTCCCTTCAGTCCA | | |
| GCCTGTGCCAGGTTCACTGCCGTGT | | |
| AGGCAGCTAAGAAA | | |
| gBlocks for cloning of | This study | Sec Table S6 |
| aca2, aca3, and | | |
| associated promoter | | |
| regions | | |

Software and Algorithms

| | | |
|---|---|---|
| Prism 7.0 | GraphPad | graphpad.com/scientific- |
| | | software/prism |
| Image Lab 6.0 | BioRad | bio-rad.com/en-ca/product |
| | | /image-lab-software |
| ImageJ | NIH | imagej.nih.gov/ij/index.html |
| PvMol | Schrödinger | pymol.org |
| Mascot | Matrix Science | matrixscience.com |
| Scaffold 3.0 Scaffold | Proteome Software | proteomesoftware.com/ |
| version 3.0 | | products/scaffold |
| Jalview | Jalview | jalview.org |
| CFX Manager 3.1 | BioRad | bio-rad.com/en-ca/product |
| | | /cfx-manager-software |

Other

| | | |
|---|---|---|
| CFX384 Touch Real-Time PCR Detection | BioRad | Cat#1855485 |
| System | | |

### Methods

Experimental Model and Subject Details. Microbes. Pseudomonas aeruginosa strains (UCBPP-PA14 and UCBPP-PA14 CRISPR mutant derivatives) and Escherichia coli strains (DH5a, SM10λpir, BL21(DE3)) were cultured at 37° C. in lysogeny broth (LB) or on LB agar supplemented with antibiotics at the following concentrations when appropriate: ampicillin, 100 μg mL⁻¹ for E. coli; carbenicillin, 300 μg mL⁻¹ for P. aeruginosa; gentamicin, 30 μg mL⁻¹ for E. coli and 50 μg mL⁻¹ for P. aeruginosa. Phages. Pseudomonas aeruginosa phages JBD44, JBD30 and JBD30 derivatives, DMS3 and DMS3 derivatives were propagated on PA14ΔCRISPR and stored in SM buffer (100 mM NaCl, 8 mM Mg₂SO4, 50 mM Tris-HCl pH 7.5, 0.01% w/v gelatin) over chloroform at 4° C.

Method Details. Mass spectrometry of the JBD30 virion. Mass spectrometry analysis was performed as previously described (Harvey et al., 2018). Briefly, 3.8×10⁹ phage particles from lysates were purified by cesium chloride density gradient ultracentrifugation (Sambrook and Russell, 2006) and subjected to tryptic digest (Lavigne et al., 2009). Liquid chromatography tandem-mass spectrometry spectra were collected on a linear ion-trap instrument (ThermoFisher) (SPARC BioCentre, The Hospital for Sick Children, Toronto, Canada). Proteins were identified using Mascot (Matrix Science) and analyzed in Scaffold version 3.0 (Proteome Software). The cut-off for protein identification was set at a confidence level of 95% with a requirement for at least two peptides to match a protein.

Introduction of an anti-CRISPR locus into phage JBD44. The anti-CRISPR locus of phage JBD30 was PCR amplified and cloned as a SalI restriction fragment into the transposon of pBTK30 (Goodman et al., 2004). This construct was transformed into E. coli SM10λpir. Conjugation was then used to move the transposon into a JBD44 lysogen of PA14. Following conjugation, lysogens were grown to log phase (OD₆₀₀=0.5) and prophages were induced with mitomycin C (3 μg mL⁻¹). Lysates were plated on lawns of PA14 expressing a crRNA targeting phage JBD44 from pHERD30T to isolate phages carrying and expressing the anti-CRISPR locus.

Phage plaque and spotting assays. For spotting assays, 150 μL of overnight culture was added to 4 mL of molten top agar (0.7%) supplemented with 10 mM MgSO₄ and poured over prewarmed LB agar plates containing 10 mM MgSO₄ and antibiotic as needed. After solidification of the top agar lawn, 10-fold serial dilutions of phage lysate were spotted on the surface. The plates were incubated upright overnight at 30° C.

For plaque assays, 150 μL of overnight culture was mixed with an appropriate amount of phage and incubated at 37° C. for 10 minutes. The bacteria/phage mixture was added to 4 mL of molten top agar (0.7%) supplemented with 10 mM MgSO₄ and poured over prewarmed LB agar plates containing 10 mM MgSO₄ and antibiotic as needed. The plates were incubated upright overnight at 30° C. Plaques were counted and expressed as the number of plaque forming units (PFU) mL⁻¹. Plaque sizes were analyzed using ImageJ (Schneider et al., 2012). Images of plaque assays were converted to 8-bit (grayscale). The image threshold was then adjusted to isolate plaques from the image background. The area of each plaque was measured in pixels squared. Image sizes were calibrated using the diameter of the petri dish in the image.

Phage infection time course. Overnight cultures of PA14 or PA14ΔCRISPR were subcultured 1:100 into LB and grown with shaking at 37° C. to an OD600 of 0.4. After removing 1 mL of culture for an uninfected control, phage JBD30 was added at a multiplicity of infection (MOI) of 5 or 8. Samples were removed after 0, 2, 4, 6, 8, 10, 20, 30, 40, 50, 60, and 70 minutes. Cells were pelleted and flash frozen. One round of infection was stopped at 70 minutes post phage addition. To help synchronize the infection, cells were pelleted 10 minutes post phage addition and resuspended in fresh pre-warmed LB. Lysogens were subcultured 1:100 from overnight cultures and grown for 5 hours prior to RNA extraction.

RNA extraction and RT-qPCR. Cell pellets were resuspended in 800 μL LB and mixed with 100 μL lysis buffer (40 mM sodium acetate, 1% SDS, 16 mM EDTA) and 700 μL acid phenol:chloroform pre-heated at 65° C. The mixture was incubated at 65° C. for 5 minutes with regular vortexing and centrifuged at 12,000×g for 10 minutes at 4° C. The aqueous layer was collected, extracted with chloroform, and precipitated with ethanol. Total RNA was resuspended in water and subsequently treated with DNase (TURBO DNA-free kit, Ambion) according to the manufacturer's instructions. cDNA was synthesized using SuperScript IV VILO master mix (Invitrogen) and quantified using PowerUp SYBR green master mix (Applied Biosystems) with primers listed in Table 5. For the purpose of quantification, standards were generated by PCR. Data were analyzed using BioRad CFX manager 3.1 software.

Cloning of aca genes and associated promoter regions. aca1 and its associated promoter region were PCR amplified from lysates of phage JBD30 using the primers listed in Table S2. aca1 was cloned as a NcoI/HindIII restriction fragment into pHERD30T (for anti-CRISPR activity assays in P. aeruginosa) or into BseR1/HindIII cut p15TV-L (for protein expression and purification in E. coli). The promoter region was cloned as a NcoI/HindIII restriction fragment into the promoterless β-galactosidase reporter shuttle vector pQF50 (Farinha and Kropinski, 1990).

The anti-CRISPR locus from Pectobacterium phage ZF40 (NC_019522.1: 19220-19999) and the anti-CRISPR upstream region and Aca3 coding sequence from Neisseria meningitidis strain 2842STDY5881035 (NZ_FERW01000005.1: 56624-56978; NZ_FERW01000005.1: 55654-55893) were synthesized as gBlocks (Integrated DNA Technologies). aca2 and aca3 were PCR amplified from their respective gBlocks using primers list in Table 5. Each fragment was gel purified and cloned into pCM-Str using isothermal assembly (Gibson et al., 2009). The anti-CRISPR upstream regions from ZF40 and N. meningitidis were amplified by PCR and cloned as a NcoI/HindIII restriction fragment into pQF50. All plasmids were verified by sequencing.

β-galactosidase reporter assays. β-galactosidase reporter plasmids were transformed into DH5a and PA14. Overnight cultures of transformed cells were subcultured 1:100 and grown for ˜3 hours with shaking (OD₆₀₀=0.4-0.7). β-galactosidase activity was then quantified using a method derived from Zhang and Bremer, 1995. Briefly, 20 μL of culture was mixed with 80 μL of permeabilization solution (0.8 mg mL⁻¹ CTAB, 0.4 mg mL⁻¹ sodium deoxycholate, 100 mM Na₂HPO₄, 20 mM KCl, 2 mM MgSO₄, 5.4 μL mL⁻¹β-mercaptoethanol) and incubated at 30° C. for 30 minutes. 600 μl of substrate solution (60 mM Na₂HPO₄, 40 mM NaH₂PO₄, 1 mg mL⁻¹ o-nitrophenyl-β-galactosidase) was added and the reaction was allowed to proceed at 30° C. for 30 minutes to 1.5 hours. The reaction was stopped with the addition of 700 μL of 1 M Na₂CO₃, A420 and A550 were measured, and Miller Units were calculated.

Designing and introducing Aca1 amino acid substitutions. Key residues of the Aca1 HTH domain were identified using HHPRED and modeled onto the helix-turn-helix domain of PlcR (PDB: 3U3W) using PyMol to generate a reference homology model of Aca1. Alanine substitutions at key Aca1 residues were introduced by site-directed mutagenesis with Phusion polymerase (Thermo Scientific) in either pHERD30T (for P. aeruginosa activity assays) or p15TV-L (for protein expression and purification in E. coli).

Purification of Aca1 proteins. Overnight cultures of E. coli BL21(DE3) carrying the appropriate Aca1 expression plasmid were subcultured 1:100 and grown with shaking at 37° C. to an OD600 of 0.5. Protein expression was induced with 1 mM IPTG for 4 hours at 37° C. Cells were lysed by sonication in binding buffer (20 mM Tris-HCl pH 7.5, 250 mM NaCl, 5 mM imidazole). Clarified lysates were batch bound to Ni-NTA agarose resin (Qiagen) at 4° C. for 1 hour, passed through a column at room temperature, and washed extensively with binding buffer containing 30 mM imidazole. Bound protein was eluted with binding buffer containing 250 mM imidazole and dialyzed overnight at 4° C. in buffer containing 10 mM Tris-HCl pH 7.5 and 150 mM NaCl. All Aca1 mutant purified at levels similar to wild-type. Proteins were purified to greater than 95% homogeneity as assessed by Coomassie-stained SDS-PAGE.

Electrophoretic mobility shift assay. Varying concentrations of purified Aca1 or Aca1 mutants were mixed with 20 ng of target DNA (gel purified PCR product or annealed oligo) in binding buffer (10 mM HEPES pH 7.5, 1 mM MgCl₂, 20 mM KCl, 1 mM TCEP, 6% v/v glycerol) and incubated on ice for 20 minutes. The DNA-protein complexes were separated by gel electrophoresis at 100 V on a 6% native 0.5× TBE polyacrylamide gel. Gels were stained at room temperature with Sybr gold (Invitrogen) and visualized according to the supplier's instructions. Bands were quantified using Image Lab 6.0 software (BioRad). The percent DNA bound was plotted as a function of Aca1 concentration in Prism 7.0 (GraphPad).

Annealed oligos were generated by mixing complementary oligonucleotides in a 1:1 molar ratio in annealing buffer (10 mM Tris, pH 7.5, 50 mM NaCl, 1 mM EDTA), heating at 95° C. for 5 minutes, and cooling slowly to room temperature.

Operator and Aca1 mutant phage construction. Point mutations were introduced into each inverted repeat of the anti-CRISPR promoter on a recombination cassette (JBD30 genes 34 to 38; Bondy-Denomy et al., 2013) by site-directed mutagenesis using primers listed in Table 5. Alanine substitutes of key Aca1 residues were introduced into the wild-type JBD30 recombination cassette by site-directed mutagenesis. Mutant phages were then generated using in vivo recombination as previously described by Bondy-Denomy et al., 2013. All mutations were verified by sequencing.

Construction of a JBD30 mutant phage bearing an anti-CRISPR promoter deletion. A recombination cassette consisting of genes 34 to 38 of phage JBD30 (anti-CRISPR locus with large flanking regions) in plasmid pHERD20T was previously generated (Bondy-Denomy et al., 2013). This plasmid was linearized by PCR using primers that excluded the anti-CRISPR promoter, and then re-circularized using In-fusion HD technology (Clontech) to generate a recombination cassette with an anti-CRISPR promoter deletion. Using this cassette, mutant phages were generated as previously described (Bondy-Denomy et at, 2013).

Lysogen construction. P. aeruginosa lysogens were generated by either streaking out cells to single colonies from the center of a phage-induced zone of clearing or by plating cells infected with phage and isolating single colonies. The presence of a prophage was confirmed by resistance to superinfection from the phage used to generate the lysogen.

Bioinformatics. Protein sequence similarity searches were performed with PSI-BLAST (Altschul et al., 1997). Protein sequence alignments were performed with MAFFT (Katoh et al., 2002), and nucleotide sequence alignments were performed with ClustalO (Sievers et al., 2011). HHPred was used to predict the location of HTH motifs (Soding et al., 2005).

Aca3 misannotation. A nucleotide alignment of several anti-CRISPR loci from Neisseria meningitidis revealed that many aca3 homologs had one to two in-frame start codons (ATG) upstream of their annotated start that would result in a N-terminal extension of 8 to 10 amino acid residues. aca3 was cloned with and without this N-terminal extension. Aca3 repressor activity was best with the inclusion of the N-terminal extension (sequence shown below with new residues in bold). Thus, this version was used in all experiments presented here. All other Aca protein sequences are as annotated.

Aca3:
MKMRRIWRAGMIDNPELGYTPANLKAIRQKYGLTQKQVAD
ITGATLSTAQKWEAAMSLKTHSDMPHTRWLLLLEYVRNL

Quantification and statistical analysis. All experiments were performed with at least three biological replicates (n >3). Statistical parameters are reported in the Figure Legends.

TABLE 2
Virion proteins of phage JBD30 detect by mass spectrometry

| JBD30 | | Protein Length | |
|---|---|---|---|
| ORF | Accession ID | (amino acids) | Function |
| 32 | YP_007392339.1 | 526 | portal protein |
| 33 | YP_007392340.1 | 428 | homolog of phage |
| | | | Mu gpF |
| 37 | YP_007392344.1 | 365 | protease/scaffold |
| 38 | YP_007392345.1 | 304 | major head |
| 41 | YP_007392348.1 | 138 | head-tail joining |
| 42 | YP_007392349.1 | 157 | tail terminator |
| 44 | YP_007392351.1 | 256 | tail tube |
| 46 | YP_007392353.1 | 1158 | tape measure |
| 47 | YP_007392354.1 | 318 | putative tail protein |
| 48 | YP_007392355.1 | 307 | putative tail protein |
| 49 | YP_007392356.1 | 567 | tail protein; phage |
| | | | lambda gpM-like |
| 50 | YP_007392357.1 | 273 | tail protein; phage |
| | | | lambda gpL-like |
| 53 | YP_007392360.1 | 735 | central fiber |
| 54 | YP_007392361.1 | 382 | putative pilus |
| | | | binding protein |

TABLE 3
List of genomes and anti-CRISPR protein identifiers used in FIG. 1

| Source | Genome ID | Anti-CRISPR ID |
|---|---|---|
| Pseudomonas | NC_008717.1 | YP_950454.1 |
| phage DMS3 | | |
| Alcanivorax sp. | NZ_LVIC01000002.1 | WP_063139756.1 |
| KX64203 | | |
| Pectobacterium | NC_019522.1 | YP_007006940.1 |
| phage ZF40 | | |
| Halomonas sinaiensis | NZ_BDEO01000016.1 | WP_064700809.1 |
| DSM 18067 | | |
| Neisseria meningitidis | NZ_OALB1000002.1 | WP_042743676.1; |
| 23231 | | WP_042743678.1 |
| Listeria monocytogenes | NC_017545.1 | WP_003722517.1; |
| J0161 | | WP_003722518.1 |
| Streptococcus | NC_000872.1 | NP_049988.1 |
| phage Sfi21 | | |
| Streptococcus | MH000604.1 | AVO22749.1 |
| phage D1811 | | |
| Sulfolobus islandicus | NC_030884.1 | YP_009272954.1 |
| rudivirus 3 | | |

TABLE 4
List of genomes and Aca protein identifiers used in FIG. 11

| Source | Genome ID | Aca ID |
|---|---|---|
| Phaseolibacter flectens | NZ_JAEE01000001.1 | WP_036985669.1 |
| Proteus penneri | GG661994.1 | EEG86165.1 |
| Shewanella xiamenensis | JGVI01000034.1 | KEK29120.1 |
| Vibrio parahaemolyticus | NZ_JPKT01000003.1 | WP_080285139.1 |
| Vibrio cyclitrophicus | KP795522.1 | AKN37111.1 |
| Pectobacterium phage ZF40 | NC_019522.1 | YP_007006939.1 |
| Oceanimonas smirnovii | NZ_KB908455.1 | WP_019933869.1 |
| Brackiella oedipodis | NZ_KK211205.1 | WP_028357637.1 |
| Nme NmSL13x2 | NZ_NGAT01000003.1 | WP_002212356.1 |
| Nme 22472 | NZ_OAFV01000002.1 | WP_002255676.1 |
| Nme M40030 | NZ_QQEW01000023.1 | WP_118803841.1 |
| Nme 2842STDY5881035 | NZ_FERW01000005.1 | WP_042743680.1 |
| Nme NM80179 | NZ_ALXV01000004.1 | WP_002231710.1 |
| Nme 2842STDY5881013 | NZ_FERN01000021.1 | WP_061695140.1 |
| Nme WUE2121 | NZ_CP012394.1 | WP_061384811.1 |

TABLE 5
Oligonucleotides used in this study

| Purpose | Sequence (5′-3′) |
|---|---|
| Cloning of JBD30 anti- | F: GGGCCCGTCGACTGGCCACT |
| CRISPR locus into | TTCGGACAAG |
| pBTK30 transposon | R: CCCGGGGTCGACTCACGCAG |
| | ATGGCGGGTCGT |
| Generation of RT-qPCR | F: TGGTTCAGCCCTCAACAACT |
| standard for gene A | R: TCTTGAGCATGGCGAGCA |
| RT-qPCR of gene A | F: GCCTCGGTTCAACAGTACGA |
| | R: AACGTGGTACTCCATCGCTTT |
| Generation of RT-qPCR | F: AGTTCGCCTTTATGGACGAG |
| standard for gene G | R: ATTTCGGCTCAAGGCTGTTA |
| RT-qPCR of gene G | F: CGGGTCCAACTTGGTCTATG |
| | R: TTTCGTCGAACGGCAGATA |
| Generation of RT-qPCR | F: ATGAAGTTCATCAAATACCTC |
| standard for acrIF1 | R: TCAGGGGTTTTCACGCCGGG |
| RT-qPCR of acrIF1 | F: AATACCTCAGCACCGCTCAC |
| | R: TTGCCGTTTACGACGTTCTC |
| Generation of RT-qPCR | F: ATGAGATTTCCCGGCGTGAA |
| standard for aca1 | R: TCACGCAGATGGCGGGTCGT |
| RT-qPCR of aca1 | F: TCAAGAAAGCCGGCATCA |
| | R: TCCTTGATGTCCTCGCTCAG |
| Generation of RT-qPCR | F: GAAAAGAACCGCCTACTCGTT |
| standard for gene 37 | R: TGGCTTTCAGGAGTTCATCC |
| (protease/scaffold) | |
| RT-qPCR of gene 37 | F: ATGAGCACCAGACCCTCAAG |
| | R: GGGCTGTGTATTCGACACG |
| Generation of RT-qPCR | F: CTGCAAGAGTTTCTGGATGATG |
| standard for clpX | R: CTTTATCTGCGACGAGTGTGTC |
| RT-qPCR of clpX | F: CGCTTGTAGTGGTTGTATACCG |
| | R: AAAGTAGTGGGCACAAACTTCC |
| Generation of RT-qPCR | F: GAGATGCGGTTGAGCTTGTT |
| standard for rpoD | R: GTCGACAGCGTCCTGAAGAG |
| RT-qPCR of rpoD | F: GGGCGAAGAAGGAAATGGTC |
| | R: CAGGTGGCGTAGGTAGAGAA |
| Cloning of anti-CRISPR | F: CCCGGGCCCCATGGTGGCCA |
| promoter from JBD30 | CTTTCGGACAAG |
| | R: CCCGGGAAGCTTGGTTTGAA |
| | TCCTTGTTGGCGCC |
| Generation anti-CRISPR | F: AGCCGAAATCGGTAGAACGG |
| promoter deletion | CGAGGCGCCAACAAG |
| recombination cassette | R: CTACCGATTTCGGCTCAAG |
| Cloning Aca1 | F: CCCGGGCCATGGCCAGATTT |
| | CCCGGCGTGAA |
| | R: CCCGGGAAGCTTTCACGCAG |
| | ATGGCGGGTCGT |
| wild-type anti-CRISPR | sense: ACAAGCGGCACACTGTG |
| promoter substrate for | CCTATTGCGAATTAGGCACAATGT |
| EMSA | GCCTAATCTAACG |
| | anti-sense: GGTTAGATTAGG |
| | CACATTGTGCCTAATTCGCAATAG |
| | GCACAGTGTGCCGCTTGT |
| IR1 mutant anti-CRISPR | sense: ACAAGCGTCGTACTGTG |
| promoter substrate for | CCTATTGCGAATTAGGCACAATGT |
| EMSA | GCCTAATCTAACG |
| | anti-sense: CGTTAGATTAGG |
| | CACATTGTGCCTAATTCGCAATAG |
| | GCACAGTACGACGCTTGT |
| IR2 mutant anti- | sense: ACAAGCGGCACACTGTG |
| CRISPR promoter | CCTATTGCGAGCTAGTCCCAATGT |
| substrate for EMSA | GCCTAATCTAACG |
| | anti-sense: CGTTAGATTAGG |
| | CACATTGGGACTAGCTCGCAATAG |
| | GCACAGTGTGCCGCTTGT |
| IR1 + IR2 mutant | sense: ACAAGCGTCGTACTGTG |
| anti-CRISPR promoter | CCTATTGCGAGCTAGTCCCAATGT |
| substrate for | GCCTAATCTAACG |
| EMSA | anti-sense: CGTTAGATTAGG |
| | CACATTGGGACTAGCTCGCAATAG |
| | GCACAGTACGACGCTTGT |
| Generation of IR1 | sense: GGCCACTTTCGGACAAG |
| mutations in | CGTCGTACTGTGCCTATTGCGAAT |
| anti-CRISPR promoter | T |
| | anti-sense: AATTCGCAATAG |
| | GCACAGTACGACGCTTGTCCGAAA |
| | GTGGCC |
| Generation of IR2 | sense: TGACGTTAGATTAGGCA |
| mutations in | CATTGGGACTGCTTCGCAATAGGC |
| anti-CRISPR promoter | ACAGTGTGCC |
| | anti-sense: GGCACACTGTGC |
| | CTATTGCGAAGCAGTCCCAATGTG |
| | CCTAATCTAACGTCA |
| Generation of | sense: TCGGCTGCGCGCGCCTG |
| R33A Aca1 | GCTGATGCC |
| mutant | anti-sense: GGCATCAGCCAG |
| | GCGCGCGCAGCCGA |
| Generation of | sense: CAGCTCGGCTGCGGCCC |
| R34A Aca1 | GCTGGCTGATG |
| mutant | anti-sense: CATCAGCCAGCG |
| | GGCCGCAGCCGAGCTG |
| Generation of | sense: CAGCTCGGCTGCGGCCG |
| R33A/R34A Aca1 | CCTGGCTGATGCCG |
| mutant | anti-sense: CGGCATCAGCCA |
| | GGCGGCCGCAGCCGAGCTG |
| Generation of | sense: GTAATAGCGCATCACCG |
| R44A Aca1 | CGTCACTGAGGCCGAGC |
| mutant | anti-sense: GCTCGGCCTCAG |
| | TGACGCGGTGATGCGCTATTAC |
| Cloning of Aca2 | F: TAGTTGCGGCCGCAAAATGGA |
| | TGAATGGTCAAGAATTAAAAAAAG |
| | R: GCGGCCGCAGGCAAAGGATAT |
| | TAGATTAAATCCGCGTGAC |
| Cloning of Aca3 | F: TAGTTGCGGCCGCAAAATGGA |
| | TGAAGAAATTTGAAGccc |
| | R: GCGGCCGCAGGCAAAGGATAT |
| | TATTTTAATGAATCCAAAAGTTTT |
| | TG |
| Amplification | F: TATCCTTTGCCTGCGGCC |
| of pCM-Str | R: CCATTTTGCGGCCGCAAC |
| for Aca cloning | |
| Cloning of Aca2 | F: CCCGGGCCATGGAGCCTCACCT |
| associated upstream | CCGGCG |
| region | R: CCCGGGAAGCTTCTCGAACCGA |
| | TGAATAAATTATATGT |
| Cloning of aca3 | F: CCCGGGCCATGGAATTGAATCC |
| associated | GCAATGGTGAAA |
| upstream region | R: CCCGGGAAGCTTTTTGAAATCC |
| | TTTCGTTTATCCTTG |

TABLE 6
Plasmids used in this study

| Plasmid ID | Purpose | Backbone |
|---|---|---|
| pES102 | Overexpression of JBD44-targeting | pHERD30T |
| | crRNA in P. aeruginosa | |
| pSY100 | Overexpression of JBD30 | pHERD30T |
| | Aca1 in P. aeruginosa | |
| pSY115 | Overexpression of R33A Aca1 | pHERD30T |
| | mutant in P. aeruginosa | |
| pSY116 | Overexpression of R34A Aca1 | pHERD30T |
| | mutant in P. aeruginosa | |
| pSY117 | Overexpression of R33A/34A | pHERD30T |
| | Aca1 mutant in P. aeruginosa | |
| pSY118 | Overexpression of R44A Aca1 | pHERD30T |
| | mutant in P. aeruginosa | |
| pSY107 | Generation of JBD30ΔPacr | pHERD20T |
| pSY108 | Generation of JBD30 IR1 mut | pHERD20T |
| pSY109 | Generation of JBD30 IR2 mut | pHERD20T |
| pSY110 | Generation of JBD30 IR1 + IR2 mut | pHERD20T |
| pSY119 | Generation of JBD30aca^{R33A} | pHERD20T |
| pSY120 | Generation of JBD30aca^{R34A} | pHERD20T |
| pSY121 | Generation of JBD30aca^{R33A/R34A} | pHERD20T |
| pSY122 | Generation of JBD30aca^{R44A} | pHERD20T |
| pSY105 | Encodes anti-CRISPR | pBTK30 |
| | locus carrying transposon | |
| pSY101 | Determining anti-CRISPR | pQF50 |
| | promoter region activity | |
| pSY102 | Determining IR1 | pQF50 |
| | mutant promoter activity | |
| pSY103 | Determining IR2 | pQF50 |
| | mutant promoter activity | |
| pSY104 | Determining IR1 + IR2 | pQF50 |
| | mutant promoter activity | |
| pSY138 | Determining aca2-associated | pQF50 |
| | promoter activity | |
| pSY139 | Determining aca3-associated | pQF50 |
| | promoter activity | |
| pSY123 | Expression and purification | p15TV-L |
| | of JBD30 Aca1 | |
| pSY124 | Expression and purification | p15TV-L |
| | of R33A Aca1 mutant | |
| pSY125 | Expression and purification | p15TV-L |
| | of R34A Aca1 mutant | |
| pSY126 | Expression and purification | p15TV-L |
| | R33A/34A Aca1 mutant | |
| pSY127 | Expression and purification | p15TV-L |
| | of R44A Aca1 mutant | |
| pSY146 | Constitutive expression of aca1 in E. coli | pCM-Str |
| pSY144 | Constitutive expression of aca2 in E. coli | pCM-Str |
| pSY145 | Constitutive expression of aca3 in E. coli | pCM-Str |

TABLE 7
Sequences for cloning of aca2, aca3, and
associated promoter regions

| | Reference | |
|---|---|---|
| Description | Sequence | Sequence |
| Anti-CRISPR | NC_ | AGCCTCACCTCCGGCGTTGCCGTGG |
| locus phage | 019522.1 | CGCTGTGTGATTTACAGGAAATAAA |
| ZF40 | | AAGGCCACGAATGCGGCCTTAGCGA |
| | | TTAAAAAATATGAAATGCCTTGCTT |
| | | GTTCGCGATTGCGAACATATAATTT |
| | | ATTCATCGGTTCGAGATGGCTCGAA |
| | | TCGCTCCTAACGAGGATTCCACAAT |
| | | GTCTACTGCTTACATCATCTTTAAC |
| | | TCATCCGTCGCGGCCGTAGTTGATA |
| | | CTGAGATCGCTAATGGCGCTAATGT |
| | | CACATTCTCAACAGTGACCGTTAAA |
| | | GAAGAAATTAACGCGAACCGTGATT |
| | | TCAATCTGGTTAACGCTCAGAACGG |
| | | GAAAATCTCACGCGCAAAACGCTGG |
| | | GGAAACGAGGCGTCAAAATGTGAGT |
| | | ATTTTGGCCGAGAAATAAACCCAAC |
| | | CGAGTTTTTCATCAAATAATGTGGT |
| | | CAAAATGACAAACAAAGAACTTCAG |
| | | GCAATCAGAAAACTGTTAATGCTGG |
| | | ATGTATCAGAAGCGGCTGAACACAT |
| | | TGGCCGCGTTTCCGCCCGGAGTTGG |
| | | CAATATTGGGAGTCTGGACGCTCTG |
| | | CTGTTCCTGATGATGTTGAGCAGGA |
| | | AATGTTGGATTTAGCGTCAGTCAGG |
| | | ATAGAAATGATGTCCGCTATAGACA |
| | | AGCGTCTCGCCGATGGCGAACGTCC |
| | | TAAATTACGTTTTTATAACAAGTTG |
| | | GATGAATACCTGGCTGACAACCCCG |
| | | ATCACAATGTGATCGGGTGGCGTCT |
| | | GAGCCAGTCTGTTGCCGCACTCTAT |
| | | TACACTGAGGGTCACGCGGATTTAA |
| | | TCTAA |
| Anti-CRISPR | NZ_ | TCCCAATTACCTGTTTGAAGCAGTA |
| promoter | FERW010 | TTTGTTTCTCAAATGACCAATTTTT |
| region and | 00005.1 | AACCAAAGGCCGCTAATGTGGCCGT |
| aca3 gene | | TTTTTTTGTTCTCATACTCTTCTAA |
| from | | TTTAGGGTCTCTGCCTCCAAGCTCC |
| Neisseria | | CGGTCTCGCCGCCGACGGCTCGGGA |
| meningitidis | | GCAGGGCATAGCCATAAAAGCTTAC |
| | | ATTGTGTGCTAGACTATATCAAACT |
| | | ACAACTACGAAAGGAAATCCGAACA |
| | | CTATGAATAAAACTTATAAAATTGG |
| | | AAAAAATGCCGGGTATGATGGCTGC |
| | | GGTCTTTGTCTTGCGGCCATTTCTG |
| | | AAAATGAAGCTATCAAAGTTAAGTA |
| | | TTTGCGCGACATTTGTCCTGATTAC |
| | | GATGGCGATGATAAAGCTGAGGATT |
| | | GGCTGAGATGGGGAACGGACAGCCG |
| | | CGTCAAAGCAGCCGCTCTTGAAATG |
| | | GAGCAGTACGCATATACGTCGGTTG |
| | | GTATGGCCTCATGTTGGGAGTTTGT |
| | | TGAACTATGAAGAAATTTGAAGCCC |
| | | CTGAAATTGGCTATACACCTGCCAA |
| | | TCTTAAAGCACTGAGAAAACAATTT |
| | | GGGCTTACACAAGCTCAGGTAGCAG |
| | | AAATTACTGGTACAAAAACCGGATA |
| | | CAGCGTCCGCAGGTGGGAAGCAGCA |
| | | ATTGATGCCAAAAATCGCGCGGATA |
| | | TGCCGCTCGTAAAATGGCAAAAACT |
| | | TTTGGATTCATTAAAATAATGA |

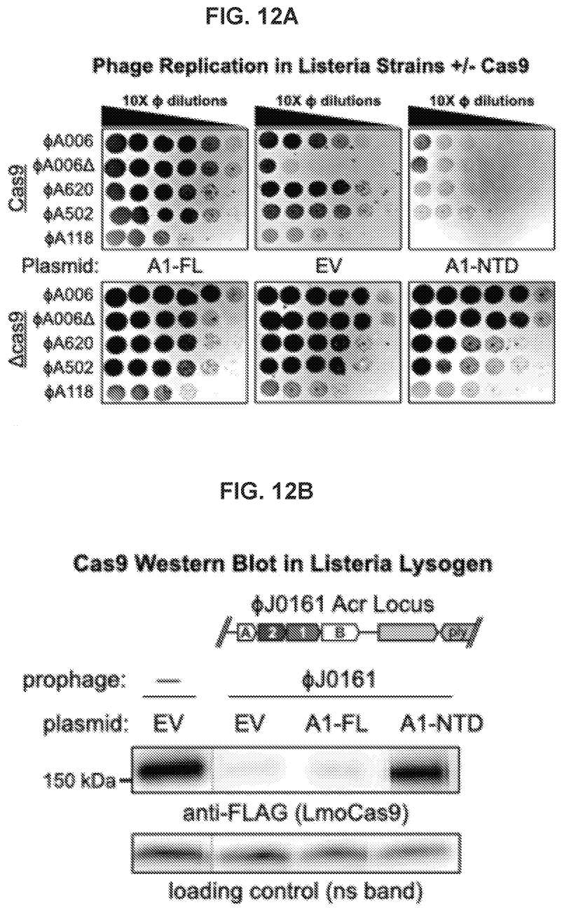

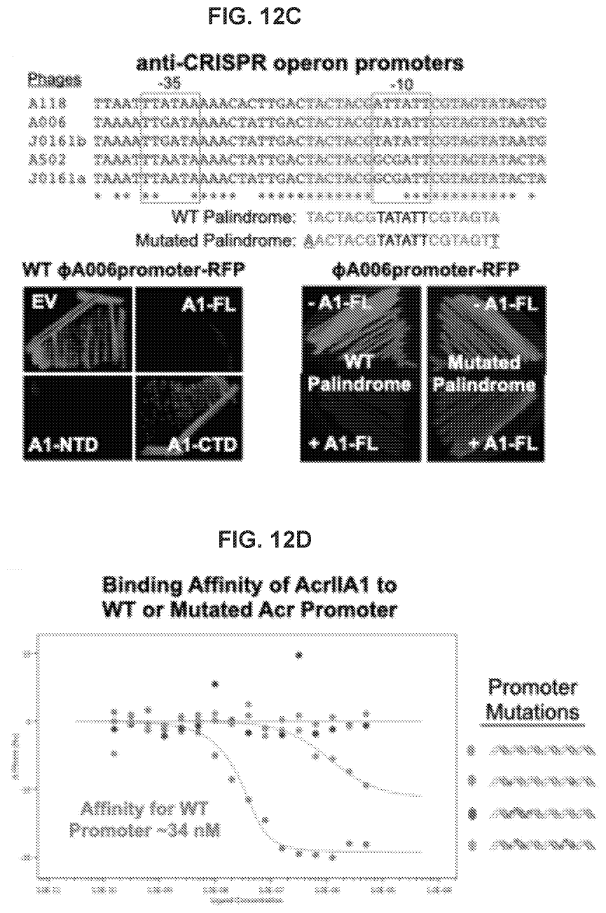

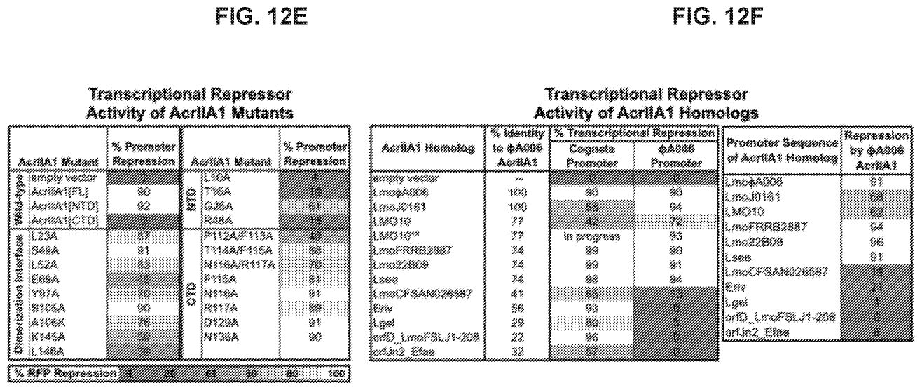

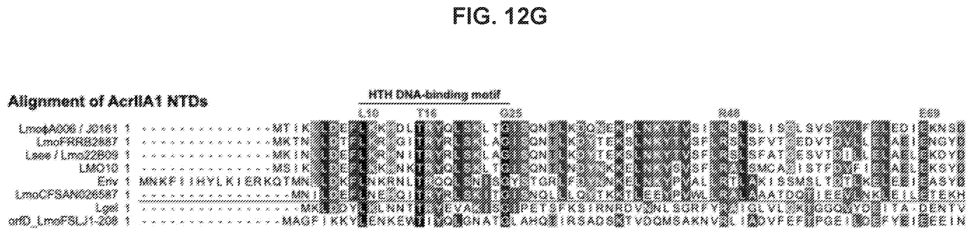

### Example 2. AcrIIA1 NTD Represses the Deployment of Anti-CRISPRs from Phages (FIG. 12A)

Four phages encoding Type II-A anti-CRISPRs were used to infect strains expressing AcrIIA1 FL (full length), the N-terminal domain (NTD), or no protein (EV) in backgrounds that contain (Cas9) or where it was knocked out, ΔCas9. Each phage replicates well in the absence of Cas9 or when the anti-CRISPR AcrIIA1 is expressed. In the presence of Cas9 EV, note that the phage with its anti-CRISPR deleted A0064 is unable to replicate as well as the phage with the anti-CRISPR (A006) or where an anti-CRISPR is expressed in trans. Moreover, we observe that the expression of the AcrIIA1 NTD (which does not possess anti-CRISPR activity) actually limits the ability of anti-CRISPR phages to deploy their anti-CRISPRs. The A1-NTD impact is dependent on Cas9, consistent with inhibiting anti-CRISPR deployment and not another aspect of phage biology.

### Example 3. Expression of the AcrIIA1 NTD can Re-Activate Cas9 that was Inhibited by Acrs (FIG. 12B)

A western blot is shown, measuring the level of Cas9 protein and a loading control in Listeria monocytogenes bacteria. In the absence of a prophage or any expressed protein, Cas9 is highly abundant (Lane 1). In lanes 2-4, a prophage is present in the strain, expressing the indicated anti-CRISPR locus, with AcrIIA1 and AcrIIA2. The expression of the AcrIIA1 anti-CRISPR causes the loss of Cas9 protein, and while EV or overexpression of A1-FL do not prevent this Cas9 loss, we observe (Lane 4) that overexpression of the A1-NTD reactivates Cas9 expression. This is due to the ability of the NTD to repress the anti-CRISPR promoter. This is not seen in the presence of A1-FL because the CTD of this protein is what mediates the Cas9 loss.

### Example 4. Phage Anti-CRISPR Promoters are Repressed by AcrIIA1-NTD (FIG. 12C)

The promoter sequences of 5 distinct anti-CRISPR Listeria phages with the binding site highlighted in yellow. The panlindrome sequence is shown below the alignment and was fused to RFP as a reporter. In the reporter, RFP is well expressed from the anti-CRISPR promoter, but repressed in the presence of AcrIIA1-FL or just the A1-NTD. When the palindrome is mutated at two positions, AcrIIA1-FL is no longer able to repress its transcription.

### Example 5. AcrIIA1 Protein Binds to the Phage Anti-CRISPR Promoter (FIG. 12D)

Raw data of a binding assay is shown, where the green line depicts the strong binding of AcrIIA1 protein to the phage anti-CRISPR promoter (34 nM binding constant). Mutations to the DNA sequence (depicted in red) weaken binding.

### Example 6. Quantification of Repressor Activity of AcrIIA1 Point Mutants (FIG. 12e)

The Acr promoter-RFP reporter construct was used to test AcrIIA1 mutants to confirm the important region of the protein responsible for DNA binding. This mutagenesis revealed key residues in the NTD required for function and also in the dimerization interface.

### Example 7. Quantification of Repressor Activity of AcrIIA1 Homologs (FIG. 12F)

Homologs of AcrIIA1 are shown, with their % seq ID to the model protein from phage A006. The ability of the protein to repress their ‘cognate promoter’ (i.e., their own endogenous promoter) or the A006 promoter is quantified. Lastly, the ability of A006 AcrIIA1 to repress the promoters from the indicated elements are indicated.

### Example 8. Key Residues in the NTD of AcrIIA1 for DNA Binding/Repression (FIG. 12G)

Protein alignment of AcrIIA1 NTD helix-turn-helix motif with key residues implicated in FIG. 12E highlighted. Note the horizontal line that depicts where the strong identity breaks, which also corresponds with lost ability of these proteins to repress the A006 promoter and vice versa.

### Example 9. Non-Limiting Lists of Exemplary Aca and AcrIIA1 Proteins

Table 8 provides a non-limiting list of exemplary Aca proteins that can be used in the present methods. The table include the amino acid sequences and accession numbers of the Acas, the names and accession numbers for their associated Acr proteins, as well as citation information, species, and information regarding sequence homology to related family members.

TABLE 8

| | | | Associ- | Associ- | | | | |
|---|---|---|---|---|---|---|---|---|
| | | | ated | ated | | | | |
| Aca | Aca | Aca | Acr | Acr | | | | SEQ |
| name | Accession | sequence | name | accession | Citation | Species | Notes | ID |
| Aca1 | YP_007392 | MRFPGVK | AcrIF1 | AcrIF1: | Bondy- | Pseudomonas | Type Aca1 | 1 |
| | 343.1 | TPDASNH | | YP_0073923 | Denomy | aeruginosa | | |
| | | DPDPRYL | | 42.1 | 2013 | phage | | |
| | | RGLLKKA | | | | JBD30 | | |
| | | GISQRRA | | | | | | |
| | | AELLGLS | | | | | | |
| | | DRVMRYY | | | | | | |
| | | LSEDIKE | | | | | | |
| | | GYRPAPY | | | | | | |
| | | TVQFALE | | | | | | |
| | | CLANDPP | | | | | | |
| | | SA | | | | | | |
| Aca1 | KSQ64855. | MQLKPRN | AcrIF4/ | AcrIF4: | Bondy- | Pseudomonas | 91% ID to | 2 |
| | 1 | TVPRPDA | AcrIE3 | KSQ64856.1, | Denomy | aeruginosa | Type Aca1 | |
| | | SSHNPDP | | AcrIE3: | 2013, | | | |
| | | RYLRGLL | | KSQ64857.1 | Pawluk | | | |
| | | KKAGISQ | | | 2014 | | | |
| | | RRAAELL | | | | | | |
| | | GLGDRVM | | | | | | |
| | | RYYLSED | | | | | | |
| | | AKDGYRP | | | | | | |
| | | APYTVQF | | | | | | |
| | | ALECLAN | | | | | | |
| | | DPPSA | | | | | | |
| Aca1 | WP_07497 | MKPDASN | AcrIE5 | AcrIE5: | Marino 2018 | Pseudomonas | 78% ID to | 3 |
| | 3302.1 | HNPDPRY | | WP_074973 | | otitidis | Type Aca1 | |
| | | LRELIER | | 300.1 | | | | |
| | | AGVSQRQ | | | | | | |
| | | AAELIGM | | | | | | |
| | | SWEGFRR | | | | | | |
| | | YLRDVDA | | | | | | |
| | | PGYRVAD | | | | | | |
| | | YRVQFAL | | | | | | |
| | | ECLAAPGT | | | | | | |
| Aca1 | SDK41238. | MPLQQRS | Cand E | Cand E: | Bondy- | Pseudomonas | 65% ID to | 4 |
| | 1 | TVRKPDA | (IC5), | SDK41378.1, | Denomy | delhiensis | Type Aca1 | |
| | | SNHNPNP | AcrIF4, | AcrIF4: | 2013, | | | |
| | | RYLRGLV | AcrIE3 | SDK41283.1, | Pawluk | | | |
| | | ERSGKSQ | | AcrIE3: | 2014, | | | |
| | | RQAAELL | | SDK41332.1 | Unpublished | | | |
| | | GLSWEGF | | | | | | |
| | | RNYLRDE | | | | | | |
| | | SHPLHRS | | | | | | |
| | | APYTVQF | | | | | | |
| | | ALECLAE | | | | | | |
| | | AE | | | | | | |
| Aca1 | OPE36160. | MKPDSSK | Cand B | Cand B: | Bondy- | Pseudomonas | 53% ID to | 5 |
| | 1 | HNPDPQY | (IC3), | KSR23770.1, | 2013, | aeruginosa | Type Aca1 | |
| | | LRGLYER | AcrIF3, | AcrIF3: | Pawluk | | | |
| | | AGLKQEE | AcrIE1 | KSR23771.1, | 2014, | | | |
| | | AARRIGI | | AcrIE1: | 2013, | | | |
| | | TARALRN | | KSR23772.1 | Unpublished | | | |
| | | YVSETAG | | | | | | |
| | | REAPYPV | | | | | | |
| | | QFALECL | | | | | | |
| | | ASES | | | | | | |
| Aca2 | YP_007006 | MTNKELQ | AcrIF8 | AcrIF8:YP_ | Pawluk | Pectobacterium | Type Aca2 | 6 |
| | 939.1 | AIRKLLM | | 007006 | 2016a | phage ZF40 | | |
| | | LDVSEAA | | 940.1 | | | | |
| | | EHIGRVS | | | | | | |
| | | ARSWQYW | | | | | | |
| | | ESGRSAV | | | | | | |
| | | PDDVEQE | | | | | | |
| | | MLDLASV | | | | | | |
| | | RIEMMSA | | | | | | |
| | | IDKRLAD | | | | | | |
| | | GERPKLR | | | | | | |
| | | FYNKLDE | | | | | | |
| | | YLADNPD | | | | | | |
| | | HNVIGWR | | | | | | |
| | | LSQSVAA | | | | | | |
| | | LYYTEGH | | | | | | |
| | | ADLI | | | | | | |
| Aca2 | WP_08028 | MPLLFRS | AcrIF9 | AcrIF9: | Pawluk | Vibrio | 42% ID to | 7 |
| | 5139.1 | FIMTNQE | | WP_031500 | 2016a | parahaemolyticus | Type Aca2 | |
| | | LKQ | | 045 | | | | |
| | | LRRLLFI | | | | | | |
| | | EVSEAAA | | | | | | |
| | | LIGEC | | | | | | |
| | | EPRTWQR | | | | | | |
| | | WEKGDRA | | | | | | |
| | | IP | | | | | | |
| | | NDVSREI | | | | | | |
| | | QMLALTR | | | | | | |
| | | LER | | | | | | |
| | | LQVEFDE | | | | | | |
| | | TDPNYRY | | | | | | |
| | | FET | | | | | | |
| | | FDEYKAY | | | | | | |
| | | GGTGNEL | | | | | | |
| | | KW | | | | | | |
| | | RLAQSVA | | | | | | |
| | | TSLLCET | | | | | | |
| | | EADK | | | | | | |
| | | WREEETI | | | | | | |
| | | D | | | | | | |
| Aca2 | KEK29 | MTNTELK | AcrIF10 | AcrIF10: | Pawluk | Shewanella | 41% ID to | 8 |
| | 120.1 | QLRTLLF | | WP_037415 | 2016a | xiamenensis | Type Aca2 | |
| | | LDVTEAA | | 910.1 | | | | |
| | | QHIGDCE | | | | | | |
| | | PRTWQRW | | | | | | |
| | | EKGDRAV | | | | | | |
| | | PVDVAQT | | | | | | |
| | | MQMLALT | | | | | | |
| | | RVDMLQV | | | | | | |
| | | EYDAADP | | | | | | |
| | | MYQYFSE | | | | | | |
| | | YEDFKAA | | | | | | |
| | | TGATGAS | | | | | | |
| | | VLKWRLA | | | | | | |
| | | QSVSAQL | | | | | | |
| | | VSEQQAE | | | | | | |
| | | IWRAEET | | | | | | |
| | | I | | | | | | |
| Aca2 | WP_02835 | MNGQELK | AcrIC1 | AcrIC1: | Pawluk | Brackiella | 44% ID to | 9 |
| | 7637.1 | KARALLN | | WP_028357 | 2016b | oedipodis | Type Aca2 | |
| | | LSQQEAA | | 638.1 | | | | |
| | | KLIGDVS | | | | | | |
| | | KRSWVFW | | | | | | |
| | | ESGRPSI | | | | | | |
| | | PQDVQEK | | | | | | |
| | | FNDLLMR | | | | | | |
| | | RKAIVQP | | | | | | |
| | | FIDKTIS | | | | | | |
| | | PSNVYRI | | | | | | |
| | | YLDQNDL | | | | | | |
| | | AFISDPI | | | | | | |
| | | ELRLLQG | | | | | | |
| | | VALTLHF | | | | | | |
| | | DYDLPLV | | | | | | |
| | | DFDMKDY | | | | | | |
| | | EQWLQDQ | | | | | | |
| | | DKTDDPT | | | | | | |
| | | TR | | | | | | |
| | | SEWASTN | | | | | | |
| | | HPCSSKI | | | | | | |
| | | SD | | | | | | |
| Aca2 | WP_01993 | MTHYELQ | AcrIF6 | AcrIF6: | Pawluk | Oceanimonas | 50% ID to | 10 |
| | 3869.1 | ALRKLLM | | WP_019933 | 2016a | smirnovii | Type Aca2 | |
| | | LEVSEAA | | 870.1 | | | | |
| | | REIGDVS | | | | | | |
| | | PRSWQYW | | | | | | |
| | | ESGRSPV | | | | | | |
| | | PDDVANQ | | | | | | |
| | | IRNLTDM | | | | | | |
| | | RYQLLEL | | | | | | |
| | | RTEQIEK | | | | | | |
| | | AGKPIQL | | | | | | |
| | | NFYRTLD | | | | | | |
| | | DYEAVTG | | | | | | |
| | | KRDWSWR | | | | | | |
| | | LTQAVAA | | | | | | |
| | | TLFAEGD | | | | | | |
| | | VTLVEQG | | | | | | |
| | | GLTLE | | | | | | |
| Aca3 | WP_04274 | MKMRRIW | AcrIIC2/ | AcrIIC2: | Pawluk | Neisseria | Type Aca3 | 11 |
| | 3680.1 | RAGMIDN | AcrIIC3 | WP_04274 | 2016b | meningitidis | | |
| | | PELGYTP | | 3678.1 | | 2842STDY5881035 | | |
| | | ANLKAIR | | AcrIIC3: | | | | |
| | | QKYGLTQ | | WP_04274 | | | | |
| | | KQVADIT | | 3676.1 | | | | |
| | | GATLSTA | | | | | | |
| | | QKWEAAM | | | | | | |
| | | SLKTHSD | | | | | | |
| | | MPHTRWL | | | | | | |
| | | LLLEYVR | | | | | | |
| | | NL | | | | | | |
| Aca4 | WP_07938 | MTPDQFD | AcrIF11 | AcrIF11: | Marino | Pseudomonas | | 12 |
| | 1596.1 | ALAELIR | | WP_034011 | 2018 | aeruginosa | | |
| | | LRGGASQ | | 523.1 | | | | |
| | | EAARLVL | | | | | | |
| | | VDGMSPS | | | | | | |
| | | DAARQVE | | | | | | |
| | | ASPOAVS | | | | | | |
| | | NVLASCR | | | | | | |
| | | RGLALVL | | | | | | |
| | | RASGKGA | | | | | | |
| | | TA | | | | | | |
| Aca4 | WP_02308 | MTKEQFS | AcrIF12 | AcrIF12: | Marino | Pseudomonas | | 13 |
| | 6532.1 | ALAELMR | | WP_023086 | 2018 | aeruginosa | | |
| | | LRGGPGQ | | 531.1 | | | | |
| | | DAARLVL | | | | | | |
| | | VNGLKPT | | | | | | |
| | | EAARQTG | | | | | | |
| | | ITPQAVN | | | | | | |
| | | KTLSSCR | | | | | | |
| | | RGIELAK | | | | | | |
| | | RVFT | | | | | | |
| Aca4 | EWC40190. | MMTGEQF | Cand | CandJZ36: | Marino | Pseudomonas | | 14 |
| | 1 | GALAELL | JZ36 | EWC40191.1 | 2018, | stutzeri | | |
| | | RLRGGAS | (IC6) | | Unpublished | | | |
| | | QEAARLV | | | | | | |
| | | LVEGLAP | | | | | | |
| | | AEAARQA | | | | | | |
| | | GTTPQAV | | | | | | |
| | | SNALASC | | | | | | |
| | | RRGLELA | | | | | | |
| | | RVAAG | | | | | | |
| Aca4 | WP_10119 | MTAEQFS | Cand | CandJZ36: | Marino | Pseudomonas sp. | | 15 |
| | 2670.1 | ALAELLR | JZ36 | WP_101192 | 2018, | | | |
| | | LRGGASQ | (IC6) | 669.1 | Unpublished | | | |
| | | EAARLVL | | | | | | |
| | | VEQLTPA | | | | | | |
| | | EAARAAG | | | | | | |
| | | CSPQAVS | | | | | | |
| | | NVLASCR | | | | | | |
| | | RGLELAH | | | | | | |
| | | AAVGH | | | | | | |
| Aca5 | WP_05010 | MPLIEYI | AcrIF11 | AcrIF11: | Marino 2018 | Yersinia | | 16 |
| | 1207.1 | RLTFSGN | | WP_050101 | | frederiksenii | | |
| | | KSEFARH | | 208.1 | | | | |
| | | MGVDRQK | | | | | | |
| | | VQVWIKG | | | | | | |
| | | EWIVVGN | | | | | | |
| | | KLYAPRR | | | | | | |
| | | DIPDIRL | | | | | | |
| | | DTVSQRL | | | | | | |
| | | D | | | | | | |
| Aca5 | WP_01256 | MNKMNAR | AcrIF11 | AcrIF11: | Marino 2018 | Escherichia | | 17 |
| | 5004.1 | TLSDYIA | | WP_000765 | | coli | | |
| | | FYHNGNQ | | 122.1 | | | | |
| | | AEFARHM | | | | | | |
| | | GVNRQQV | | | | | | |
| | | TKWIKGG | | | | | | |
| | | WIVINHQ | | | | | | |
| | | LFSPQRD | | | | | | |
| | | IPENISH | | | | | | |
| | | G | | | | | | |
| | | GSAL | | | | | | |
| Aca5 | WP_07403 | MNNDNLV | AcrIF11 | AcrIF11: | Marino 2018 | Serratia | | 18 |
| | 2234.1 | SGRTLLG | | WP_074032 | | fonticola | | |
| | | YINIFHN | | 235.1 | | | | |
| | | GSQADFA | | | | | | |
| | | RHMDVTP | | | | | | |
| | | QQVTKWI | | | | | | |
| | | SGEWIVV | | | | | | |
| | | NHQLFSP | | | | | | |
| | | KRDVPEN | | | | | | |
| | | ISGGESA | | | | | | |
| | | GN | | | | | | |
| Aca5 | WP_05708 | MKLSEFI | AcrIF11 | AcrIF11: | Marino 2018 | Dickeya solani | | 19 |
| | 3779.1 | DTEFSGS | | WP_057083 | | | | |
| | | RAEFARL | | 778.1 | | | | |
| | | MGVRPQK | | | | | | |
| | | VNDWLVA | | | | | | |
| | | GMIIHID | | | | | | |
| | | ENGQAFL | | | | | | |
| | | CSVRRDI | | | | | | |
| | | PAWNRKT | | | | | | |
| | | NFA | | | | | | |
| Aca5 | WP_03955 | MSLTEYI | AcrIF11 | AcrIF11: | Marino 2018 | Pectobacterium | | 20 |
| | 8032.1 | DKNFGGN | | WP_039558 | | carotovorum | | |
| | | KAAFARH | | 031.1 | | | | |
| | | MGVDAQA | | | | | | |
| | | VNKWIKS | | | | | | |
| | | EWFVSTT | | | | | | |
| | | DDNKIYL | | | | | | |
| | | SSARREI | | | | | | |
| | | PPLK | | | | | | |
| Aca5 | WP_07205 | MNARTLS | AcrIF11 | AcrIF11: | Marino 2018 | Enterobacter | | 21 |
| | 0017.1 | DYIEFYH | | WP_045331 | | cloacae complex | | |
| | | NGNQSDF | | 704.1 | | | | |
| | | ARHMGVN | | | | | | |
| | | RQQVTKW | | | | | | |
| | | LNGGWVV | | | | | | |
| | | INHQLYS | | | | | | |
| | | PQRDVPE | | | | | | |
| | | FVTGGGS | | | | | | |
| | | AL | | | | | | |
| Aca5 | WP_03949 | MSLTEYI | AcrIF11 | AcrIF11: | Marino 2018 | Pectobacterium | | 22 |
| | 4319.1 | DKNFAGN | | WP_039494 | | carotovorum | | |
| | | KAAFARH | | 318.1 | | | | |
| | | MGVDAQA | | | | | | |
| | | VNKWIKS | | | | | | |
| | | EWFVSTT | | | | | | |
| | | DDNKIYL | | | | | | |
| | | SSVRREI | | | | | | |
| | | PPVA | | | | | | |
| Aca6 | WP_03545 | MTAMKEW | AcrIF11 | AcrIF11: | Marino 2018 | Alcanivorax sp. | | 23 |
| | 0933.1 | RARMGWS | | WP_026949 | | | | |
| | | QRRAAQE | | 101.1 | | | | |
| | | LGVTLPT | | | | | | |
| | | YQSWEKG | | | | | | |
| | | IRLSDGS | | | | | | |
| | | PIDPPLT | | | | | | |
| | | ALLAAAA | | | | | | |
| | | REKGLPP | | | | | | |
| | | IS | | | | | | |
| Aca6 | WP_06313 | MTAMKDW | AcrIF11 | AcrIF11: | Marino 2018 | Alcanivorax sp. | | 24 |
| | 9755.1 | RTRMGWS | | WP_063139 | | | | |
| | | QRRAAQE | | 756.1 | | | | |
| | | LGVTLPT | | | | | | |
| | | YQSWERG | | | | | | |
| | | VRLSDGS | | | | | | |
| | | LIDPPLT | | | | | | |
| | | ALLAAAA | | | | | | |
| | | REKGLDP | | | | | | |
| | | I | | | | | | |
| Aca7 | WP_06470 | M1DARKH | AcrIF11 | AcrIF11: | Marino 2018 | Halomonas | | 25 |
| | 2654.1 | YDPNLAP | | WP_064702 | | caseinilytica | | |
| | | ELVRRAL | | 655.1 | | | | |
| | | AVTGTQK | | | | | | |
| | | ELAERLD | | | | | | |
| | | VSRTYLQ | | | | | | |
| | | LLGKGQK | | | | | | |
| | | SMSYAVQ | | | | | | |
| | | VMLEQVI | | | | | | |
| | | QDGET | | | | | | |
| Aca7 | WP_06470 | MIDARKY | AcrIF11 | AcrIF11: | Marino 2018 | Halomonas | | 26 |
| | 0810.1 | YNPDLAP | | WP_064700 | | sinaiensis | | |
| | | ELVSRAL | | 809.1 | | | | |
| | | AVTGTQK | | | | | | |
| | | ELAERLD | | | | | | |
| | | VSRIYIQ | | | | | | |
| | | LLGKGQK | | | | | | |
| | | TMSYAVQ | | | | | | |
| | | VMLEQVI | | | | | | |
| | | QGGEN | | | | | | |
| OrfB | WP_04975 | MPIKDLT | Cand E | Cand E: | Rauch 2016, | | | |
| | 4274.1 | GMRFGRL | (AcrIC5) | WP_012802 | Unpublished | | | |
| | | WKEATSR | | 672.1 | | | | |
| | | RTSDGNV | | | | | | |
| | | IWRCQCD | | | | | | |
| | | CGNVTEV | | | | | | |
| | | PGHSLTR | | | | | | |
| | | GNTRSCG | | | | | | |
| | | CGEEENR | | | | | | |
| | | RESGNNR | | | | | | |
| | | NKAVVKE | | | | | | |
| | | HSRADSF | | | | | | |
| | | LSPKPRA | | | | | | |
| | | DTTLGIR | | | | | | |
| | | GILRRPS | | | | | | |
| | | GRYAARI | | | | | | |
| | | TFKGKTT | | | | | | |
| | | CLGTYDS | | | | | | |
| | | LEEAANA | | | | | | |
| | | RREAEIE | | | | | | |
| | | IFDPYLI | | | | | | |
| | | ANGLPPT | | | | | | |
| | | SEEEWQK | | | | | | |
| | | ILARALE | | | | | | |
| | | KEKDNAD | | | | | | |
| | | TSTKARP | | | | | | |
| | | GKIRARK | | | | Cryptobacterium | | |
| | | NKAVQN | | | | curtum | | 27 |

Table 9 provides a non-limiting list of exemplary AcrIIA1 proteins that can be used in the present methods. The table include the amino acid sequences and accession numbers of the AcrIIA1s, the names and accession numbers for their associated Acr proteins, as well as citation information and species.

TABLE 9

| DNA- | | Amino | | | | | | Autoreg. | |
|---|---|---|---|---|---|---|---|---|---|
| binding | | Acid | Associated | Associated | | | | Function | |
| Protein | Acces | Se- | Acr | Acr | | | | Experim. | SEQ |
| Name | sion # | quence | name | accession | Citation | Species | Notes | Confirmed? | ID |
| AcrIIA1_ | WP_0 | MTIKLLD | AcrIIA2 | AcrIIA2: | Rauch 2016 | Listeria | Type | Yes | 50 |
| LmoJ0 | 03722 | EFLKKHD | | WP_00372251 | | monocytogenes | AcrIIA1 | | |
| 161 | 518.1 | LTRYQLS | | 7.1 | | | | | |
| | | KLTGISQ | | | | | | | |
| | | NTLKDQN | | | | | | | |
| | | EKPLNKY | | | | | | | |
| | | TVSILRS | | | | | | | |
| | | LSFVTGL | | | | | | | |
| | | SVSDVLF | | | | | | | |
| | | ELEDIEK | | | | | | | |
| | | NSDDLAG | | | | | | | |
| | | FKHLLDK | | | | | | | |
| | | YKLSFPA | | | | | | | |
| | | QEFELYC | | | | | | | |
| | | LIKEFES | | | | | | | |
| | | ANIEVLP | | | | | | | |
| | | FTFNRFE | | | | | | | |
| | | NEEHVNI | | | | | | | |
| | | KKDVCKA | | | | | | | |
| | | LENAITV | | | | | | | |
| | | LKEKKNE | | | | | | | |
| | | LL* | | | | | | | |
| AcrIIA1_ | KUG3 | MSIKLLD | AcrIIA3 | AcrIIA3: | Osuna | Listeria | 77% ID | Yes | 51 |
| LMO10 | 7233. | EFLKKHN | | WP_01493093 | 2019, | monocytogenes | to Type | | |
| | 1 | KTRYQLS | | 1.1 | unpublished | | AcrIIA1 | | |
| | | KLTGISQ | | | | | | | |
| | | NTLNDYN | | | | | | | |
| | | KKELNKY | | | | | | | |
| | | SVSFLRA | | | | | | | |
| | | LSMCAGI | | | | | | | |
| | | STFDVFI | | | | | | | |
| | | ELAELEK | | | | | | | |
| | | SYDDLAG | | | | | | | |
| | | FKYLLDK | | | | | | | |
| | | HKLSFPT | | | | | | | |
| | | QEFELYC | | | | | | | |
| | | LIKEFES | | | | | | | |
| | | ANIEVLP | | | | | | | |
| | | FTFNRFE | | | | | | | |
| | | NETHADI | | | | | | | |
| | | EKDVKKA | | | | | | | |
| | | LNNAIAV | | | | | | | |
| | | LEEKKRR | | | | | | | |
| | | TVIKTID | | | | | | | |
| | | YYDYS* | | | | | | | |
| AcrIIA1_ | WP_0 | MNILDEF | AcrIIA2, | AcrIIA2: | Osuna | Listeria | 41% ID | Yes | 52 |
| LmoCFS | 61665 | LNEHQIT | orfJ | WP_07794954 | 2019, | monocytogenes | to Type | | |
| AN0265 | 673.1 | RYRLSKI | | 5.1, orfJ: | unpublished | | AcrIIA1 | | |
| 87 | | TGISNQL | | WP_06166567 | | | | | |
| | | LLQYTKK | | 4.1 | | | | | |
| | | TLEEYPV | | | | | | | |
| | | WLLRALA | | | | | | | |
| | | AATDQTI | | | | | | | |
| | | EEVLNKL | | | | | | | |
| | | EILETEK | | | | | | | |
| | | HQLYGIR | | | | | | | |
| | | SFLEKYN | | | | | | | |
| | | CSFPQEE | | | | | | | |
| | | WMLYRAL | | | | | | | |
| | | YLVEALN | | | | | | | |
| | | MDLEEMK | | | | | | | |
| | | FDRFEKE | | | | | | | |
| | | EHANIEK | | | | | | | |
| | | DVQEAVS | | | | | | | |
| | | NAVSTID | | | | | | | |
| | | MIRRKKL | | | | | | | |
| | | KGHFKN* | | | | | | | |
| AcrIIA1_ | WP_0 | MKTNLLD | AcrIIA2 | AcrIIA2: | Osuna | Listeria | 74% ID | Yes | 53 |
| LmoFR | 85696 | TFLKRHG | | WP_00991764 | 2019, | monocytogenes | to Type | | |
| RB2887 | 370.1 | ITRYRLS | | 3.1 | unpublished | | AcrIIA1 | | |
| | | KLAGISQ | | | | | | | |
| | | NTLKDYT | | | | | | | |
| | | EKSLNKY | | | | | | | |
| | | TVSFLRS | | | | | | | |
| | | LSFVTGE | | | | | | | |
| | | DVTDVLL | | | | | | | |
| | | ELAEIEN | | | | | | | |
| | | GYDDLAG | | | | | | | |
| | | FKYLLDK | | | | | | | |
| | | YKLSFPA | | | | | | | |
| | | LEFELYC | | | | | | | |
| | | IIKEFES | | | | | | | |
| | | ANIEISP | | | | | | | |
| | | FTFNRFE | | | | | | | |
| | | NETHVDI | | | | | | | |
| | | EKDVKKA | | | | | | | |
| | | LQNAVTV | | | | | | | |
| | | LEERKEE | | | | | | | |
| | | LL* | | | | | | | |
| AcrIIA1_ | EFS02 | MKINLLD | AcrIIA2 | AcrIIA2: | Osuna | Listeria | 74% ID | Yes | 54 |
| Lsee | 359.1 | EFLKRHN | | EFS02 358.1 | 2019, | seeligeri | to Type | | |
| | | ITRYRLS | | | unpublished | | AcrIIA1 | | |
| | | KLAGISQ | | | | | | | |
| | | NTLKDYT | | | | | | | |
| | | EKSLNKY | | | | | | | |
| | | TVSFLRS | | | | | | | |
| | | LSFATGE | | | | | | | |
| | | SVTDILL | | | | | | | |
| | | ELAELEK | | | | | | | |
| | | DYDDLAG | | | | | | | |
| | | FKYLLDK | | | | | | | |
| | | YKLAFPA | | | | | | | |
| | | LEFELYC | | | | | | | |
| | | LIKEFES | | | | | | | |
| | | ANIEISP | | | | | | | |
| | | FTFNRFE | | | | | | | |
| | | SETHTDI | | | | | | | |
| | | EKDVKKA | | | | | | | |
| | | LQNAVTV | | | | | | | |
| | | LEERKEE | | | | | | | |
| | | LL* | | | | | | | |
| AcrIIA1_ | WP_0 | MNKFIIH | AcrIIA1 | AcrIIA1: | Osuna | Enterocoecus | 56% ID | Yes | 55 |
| Eriv | 69698 | YLKIERK | (self) | WP_06969859 | 2019, | rivorum | to Type | | |
| | 591.1 | QTMNLLD | | 1.1 | unpublished | | AcrIIA1 | | |
| | | KFLNKRN | | | | | | | |
| | | LTRQQLS | | | | | | | |
| | | NISGYST | | | | | | | |
| | | GRLFDYN | | | | | | | |
| | | NKELNKY | | | | | | | |
| | | PVALLRT | | | | | | | |
| | | LAKISSM | | | | | | | |
| | | SLTDTLK | | | | | | | |
| | | ELEEIEA | | | | | | | |
| | | SYDSLLG | | | | | | | |
| | | FRKLLEQ | | | | | | | |
| | | YELSFPD | | | | | | | |
| | | LEFELYC | | | | | | | |
| | | TIKDLES | | | | | | | |
| | | LKVKVEP | | | | | | | |
| | | FTFNRFE | | | | | | | |
| | | EEGHNNI | | | | | | | |
| | | ASDCRKA | | | | | | | |
| | | MENAISM | | | | | | | |
| | | LSEALEN | | | | | | | |
| | | VRKGKAP | | | | | | | |
| | | FEDEEI* | | | | | | | |
| AcrIIA1_ | CUR6 | MKLDDYL | AcrIIA1 | AcrIIA1: | Osuna | Leuconostoc | 29% ID | Yes | 56 |
| LgeI | 3869. | KLNNTTR | (self) | CUR63869.1 | 2019, | gelidum | to Type | | |
| | 1 | YEVAKIS | | | unpublished | | AcrIIA1 | | |
| | | GIPETSF | | | | | | | |
| | | KSIRNRD | | | | | | | |
| | | VNNLSGR | | | | | | | |
| | | FYRAIG | | | | | | | |
| | | LVLGKT | | | | | | | |
| | | GGQVYDE | | | | | | | |
| | | ITADENT | | | | | | | |
| | | VFNFLGK | | | | | | | |
| | | HHIHDKE | | | | | | | |
| | | RVTELLD | | | | | | | |
| | | YMLYFKK | | | | | | | |
| | | HDIDVTN | | | | | | | |
| | | VSFNRFE | | | | | | | |
| | | NEIENGH | | | | | | | |
| | | ILGDEDD | | | | | | | |
| | | VLQVIDN | | | | | | | |
| | | LIESFKT | | | | | | | |
| | | MKENVEA | | | | | | | |
| | | GNLPTPE | | | | | | | |
| | | KMD* | | | | | | | |
| orfB_L | WP_0 | MNNHVID | AcrIIA1, | AcrIIA1: | Rauch 2016 | Listeria | | No | 57 |
| moJ016 | 03722 | LTNKKFG | AcrIIA2 | WP_00372251 | | monocytogenes | | | |
| 1 | 519.1 | RLTVKEF | | 8.1, AcrIIA2: | | | | | |
| | | VRSENGN | | WP_00372251 | | | | | |
| | | ALWNCFC | | 7.1 | | | | | |
| | | VCGNEKE | | | | | | | |
| | | VLAQHLK | | | | | | | |
| | | RGHVQSC | | | | | | | |
| | | GCLARDN | | | | | | | |
| | | GRKHADK | | | | | | | |
| | | NLRSETA | | | | | | | |
| | | QKNALKR | | | | | | | |
| | | KLEVDAV | | | | | | | |
| | | DGTMKSA | | | | | | | |
| | | LTRSLSA | | | | | | | |
| | | RNKSGIK | | | | | | | |
| | | GVRWDEK | | | | | | | |
| | | RNKWEAS | | | | | | | |
| | | ITFQKKL | | | | | | | |
| | | HFLGRFE | | | | | | | |
| | | KKDDAVK | | | | | | | |
| | | ARRDAED | | | | | | | |
| | | KYFKPIL | | | | | | | |
| | | DKMN* | | | | | | | |
| orfD_L | EHY6 | MKGFLKR | AcrIIA4 | AcrIIA4: | Rauch 2016 | Listeria | 30% ID | No | 58 |
| moFSLJ | 1391. | YAQEKKG | | EHY61390.1 | | monocytogenes | to Type | | |
| 1-208 | 1 | WSLYKLA | | | | | AcrIIA1 | | |
| | | KESGIQD | | | | | N- | | |
| | | TTLSFAN | | | | | terminal | | |
| | | SKSVHNI | | | | | domain | | |
| | | SAINIKL | | | | | | | |
| | | ISEAVGE | | | | | | | |
| | | TPGTVLD | | | | | | | |
| | | ELTELEK | | | | | | | |
| | | EMEMETT | | | | | | | |
| | | YWYNEGT | | | | | | | |
| | | GTLLTWK | | | | | | | |
| | | EYKAKIE | | | | | | | |
| | | SEARDWL | | | | | | | |
| | | EDLQEEE | | | | | | | |
| | | EELDDSD | | | | | | | |
| | | KTSLETL | | | | | | | |
| | | VQLSFEN | | | | | | | |
| | | ESDFVLS | | | | | | | |
| | | DSEGNPI | | | | | | | |
| | | KEW* | | | | | | | |
| orfD_ϕ | YP_00 | MNELRSL | AcrIIA4 | AcrIIA4: | Rauch | Listeria | | No | 59 |
| P70 | 69059 | EMSINAK | | YP_00690594 | 2016, | phage | | | |
| | 40.1 | DYATRLE | | | unpublished | | | | |
| | | SGEGSLY | | | | | | | |
| | | IRFGDSE | | | | | | | |
| | | DYPVHAS | | | | | | | |
| | | TNSTIKE | | | | | | | |
| | | TFIELFK | | | | | | | |
| | | NGWNGYE | | | | | | | |
| | | EDEQELA | | | | | | | |
| | | EDMQEIA | | | | | | | |
| | | QELILEE | | | | | | | |
| | | LTDIFEE | | | | | | | |
| | | YEFSTDE | | | | | | | |
| | | IDTDLFS | | | | | | | |
| | | GFTFHVD | | | | | | | |
| | | MDNDEAV | | | | | | | |
| | | YLMDAIN | | | | | | | |
| | | ATKYFEA | | | | | | | |
| | | RPSSWYA | | | | | | | |
| | | LLEVSYC | | | | | | | |
| | | G* | | 0.1 | Mahendra | ϕP70 | | | |
| orfJn2_ | WP_00 | MKGLLEL | orfJ, | orfJ: | 2019, | Enterocoecus | Type | Yes | 60 |
| Efae | 2401 | STIDLFL | orfJn1 | WP_02518801 | unpublished | faecalis | orfJn2; | | |
| | 838.1 | KKYGITR | | 9.1, orfJn1: | | | 32% ID | | |
| | | NKVATQN | | WP_00240183 | | | to Type | | |
| | | IKHKISN | | 9.1 | | | AcrIIA1 | | |
| | | NALAQAN | | | | | N- | | |
| | | LRPVETY | | | | | terminal | | |
| | | SVKLILG | | | | | domain | | |
| | | LSEAVNE | | | | | | | |
| | | APEKVMA | | | | | | | |
| | | QLLEIEK | | | | | | | |
| | | SQTNSES | | | | | | | |
| | | AQKKEAY | | | | | | | |
| | | QFGNIIL | | | | | | | |
| | | EGILNTN | | | | | | | |
| | | RSTHEIR | | | | | | | |
| | | LVQYLGK | | | | | | | |
| | | RTLFCTY | | | | | | | |
| | | VSGVGAM | | | | | | | |
| | | NWSVSDY | | | | | | | |
| | | KEIAETL | | | | | | | |
| | | KIDDVDI | | | | | | | |
| | | RFRTSEN | | | | | | | |
| | | DQFWDVS | | | | | | | |
| | | ESYRY* | | | | | | | |

### REFERENCES

Agari, Y., Sakamoto, K., Tamakoshi, M., Oshima, T., Kuramitsu, S., and Shinkai, A. (2010). Transcription profile of Thermus thermophilus CRISPR systems after phage infection. J Mol Biol 395, 270-281.
Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs Nucleic Acids Res 25, 3389-3402.
Bair, C. L., Rifat, D., and Black, L. W. (2007). Exclusion of glucosyl-hydroxymethylcytosine DNA containing bacteriophages is overcome by the injected protein inhibitor IPI*. J Mol Biol 366, 779-789.
Barrangou, R., Fremaux, C., Deveau, H., Richards, M., Boyaval, P., Moineau, S., Romero, D. A., and Horvath, P. (2007). CRISPR provides acquired resistance against viruses in prokaryotes. Science 315, 1709-1712.
Bondy-Denomy, J., Garcia, B., Strum, S., Du, M., Rollins, M. F., Hidalgo-Reyes, Y., Wiedenheft, B., Maxwell, K. L., and Davidson, A. R. (2015). Multiple mechanisms for CRISPR-Cas inhibition by anti-CRISPR proteins. Nature 526, 136-139.
Bondy-Denomy, J., Pawluk, A., Maxwell, K. L., and Davidson, A. R. (2013). Bacteriophage genes that inactivate the CRISPR/Cas bacterial immune system. Nature 493, 429-432.
Borges, A. L., Zhang, J. Y., Rollins, M. F., Osuna, B. A., Wiedenheft, B., and Bondy-Denomy, J. (2018). Bacteriophage Cooperation Suppresses CRISPR-Cas3 and Cas9 Immunity. Cell 174, 917-925 e910.
Brouns, S. J., Jore, M. M., Lundgren, M., Westra, E. R., Slijkhuis, R. J., Snijders, A. P., Dickman, M. J., Makarova, K. S., Koonin, E. V., and van der Oost, J. (2008) Small CRISPR RNAs guide antiviral defense in prokaryotes. Science 321, 960-964.
Cady, K. C., White, A. S., Hammond, J. H., Abendroth, M. D., Karthikeyan, R. S., Lalitha, P., Zegans, M. E., and O'Toole, G. A. (2011). Prevalence, conservation and functional analysis of Yersinia and Escherichia CRISPR regions in clinical Pseudomonas aeruginosa isolates. Microbiology 157, 430-437.
Cardarelli, L., Lam, R., Tuite, A., Baker, L. A., Sadowski, P. D., Radford, D. R., Rubinstein, J. L., Battaile, K. P., Chirgadze, N., Maxwell, K. L., et al. (2010). The crystal structure of bacteriophage HK97 gp6: defining a large family of head-tail connector proteins. J Mol Biol 395, 754-768.
Chowdhury, S., Carter, J., Rollins, M. F., Golden, S. M., Jackson, R. N., Hoffmann, C., Nosaka, L., Bondy-Denomy, J., Maxwell, K. L., Davidson, A. R., et al. (2017). Structure Reveals Mechanisms of Viral Suppressors that Intercept a CRISPR RNA-Guided Surveillance Complex. Cell 169, 47-57 ell.
Datsenko, K. A., Pougach, K., Tikhonov, A., Wanner, B. L., Severinov, K., and Semenova, E. (2012). Molecular memory of prior infections activates the CRISPR/Cas adaptive bacterial immunity system. Nat Commun 3, 945.
Deltcheva, E., Chylinski, K., Sharma, C. M., Gonzales, K., Chao, Y., Pirzada, Z. A., Eckert, M. R., Vogel, J., and Charpentier, E. (2011). CRISPR RNA maturation by trans-encoded small RNA and host factor RNase III. Nature 471, 602-607.
Dong, Guo, M., Wang, S., Zhu, Y., Wang, S., Xiong, Z., Yang, J., Xu, Z., and Huang, Z. (2017). Structural basis of CRISPR-SpyCas9 inhibition by an anti-CRISPR protein. Nature 546, 436-439.
Espah Borujeni, A., Channarasappa, A. S., and Salis, H. M. (2014). Translation rate is controlled by coupled trade-offs between site accessibility, selective RNA unfolding and sliding at upstream standby sites. Nucleic Acids Res 42, 2646-2659.
Farinha, M. A., and Kropinski, A. M. (1990). Construction of broad-host-range plasmid vectors for easy visible selection and analysis of promoters. J Bacteriol 172, 3496-3499.
Garneau, J. E., Dupuis, M. E., Villion, M., Romero, D. A., Barrangou, R., Boyaval, P.,
Fremaux, C., Horvath, P., Magadan, A. H., and Moineau, S. (2010). The CRISPR/Cas bacterial immune system cleaves bacteriophage and plasmid DNA. Nature 468, 67-71.
Gibson, D. G., Young, L., Chuang, R. Y., Venter, J. C., Hutchison, C. A., 3rd, and Smith, H. O.
(2009). Enzymatic assembly of DNA molecules up to several hundred kilobases. Nat Methods 6, 343-345.
Goodman, A. L., Kulasekara, B., Rietsch, A., Boyd, D., Smith, R. S., and Lory, S. (2004). A signaling network reciprocally regulates genes associated with acute infection and chronic persistence in Pseudomonas aeruginosa. Dev Cell 7, 745-754.
Greene, A. C. (2018). CRISPR-Based Antibacterials: Transforming Bacterial Defense into Offense. Trends Biotechnol 36, 127-130.
Grenha, R., Slamti, L., Nicaise, M., Refes, Y., Lereclus, D., and Nessler, S. (2013). Structural basis for the activation mechanism of the PlcR virulence regulator by the quorum-sensing signal peptide PapR. Proc Natl Acad Sci USA 110, 1047-1052.
Guo, T. W., Bartesaghi, A., Yang, H., Falconieri, V., Rao, P., Merk, A., Eng, E. T., Raczkowski, A. M., Fox, T., Earl, L. A., et al. (2017). Cryo-EM Structures Reveal Mechanism and Inhibition of DNA Targeting by a CRISPR-Cas Surveillance Complex. Cell 171, 414-426 e412.
Harrington, L. B., Doxzen, K. W., Ma, E., Liu, J. J., Knott, G. J., Edraki, A., Garcia, B., Amrani, N., Chen, J. S., Cofsky, J. C., et al. (2017). A Broad-Spectrum Inhibitor of CRISPR-Cas9. Cell 170, 1224-1233 e1215.
Harvey, H., Bondy-Denomy, J., Marquis, H., Sztanko, K. M., Davidson, A. R., and Burrows, L. L. (2018). Pseudomonas aeruginosa defends against phages through type IV pilus glycosylation. Nat Microbiol 3, 47-52.
He, F., Bhoobalan-Chitty, Y., Van, L. B., Kjeldsen, A. L., Dedola, M., Makarova, K. S., Koonin, E. V., Brodersen, D. E., and Peng, X. (2018). Anti-CRISPR proteins encoded by archaeal lytic viruses inhibit subtype I-D immunity. Nat Microbiol 3, 461-469.
Hertveldt, K., and Lavigne, R. (2008). Bacteriophages of Pseudomonas. In Pseudomonas (Wiley-VCH Verlag GmbH & Co. KGaA), pp. 255-291.
Hynes, A. P., Rousseau, G. M., Agudelo, D., Goulet, A., Amigues, B., Loehr, J., Romero, D. A., Fremaux, C., Horvath, P., Doyon, Y., et al. (2018). Widespread anti-CRISPR proteins in virulent bacteriophages inhibit a range of Cas9 proteins. Nat Commun 9, 2919.
Hynes, A. P., Rousseau, G. M., Lemay, M. L., Horvath, P., Romero, D. A., Fremaux, C., and Moineau, S. (2017). An anti-CRISPR from a virulent streptococcal phage inhibits Streptococcus pyogenes Cas9. Nat Microbiol 2, 1374-1380.
lida, S., Streiff, M. B., Bickle, T. A., and Arber, W. (1987). Two DNA antirestriction systems of bacteriophage P1, darA, and darB: characterization of darA-phages. Virology 157, 156-166.
Juranek, S., Eban, T., Altuvia, Y., Brown, M., Morozov, P., Tuschl, T., and Margalit, H. (2012). A genome-wide view of the expression and processing patterns of Thermus thermophilus HB8 CRISPR RNAs. RNA 18, 783-794.
Ka, D., An, S. Y., Suh, J. Y., and Bae, E. (2018). Crystal structure of an anti-CRISPR protein, AcrIIA1. Nucleic Acids Res 46, 485-492.
Katoh, K., Misawa, K., Kuma, K., and Miyata, T. (2002). MAFFT: a novel method for rapid multiple sequence alignment based on fast Fourier transform. Nucleic Acids Res 30, 3059-3066.
Landsberger, M., Gandon, S., Meaden, S., Rollie, C., Chevallereau, A., Chabas, H., Buckling, A., Westra, E. R., and van Houte, S. (2018). Anti-CRISPR Phages Cooperate to Overcome CRISPR-Cas Immunity. Cell 174, 908-916 e912.
Lavigne, R., Ceyssens, P. J., and Robben, J. (2009). Phage proteomics: applications of mass spectrometry. Methods Mol Biol 502, 239-251.
Levy, A., Goren, M. G., Yosef, I., Auster, O., Manor, M., Amitai, G., Edgar, R., Qimron, U., and Sorek, R. (2015). CRISPR adaptation biases explain preference for acquisition of foreign DNA. Nature 520, 505-510.
Makarova, K. S., Wolf, Y. I., Alkhnbashi, O. S., Costa, F., Shah, S. A., Saunders, S. J., Barrangou, R., Brouns, S. J., Charpentier, E., Haft, D. H., et al. (2015). An updated evolutionary classification of CRISPR-Cas systems. Nat Rev Microbiol 13, 722-736.
Margolin, W., Rao, G., and Howe, M. M. (1989). Bacteriophage Mu late promoters: four late transcripts initiate near a conserved sequence. J Bacteriol 171, 2003-2018.
Marino, N. D., Zhang, J. Y., Borges, A. L., Sousa, A. A., Leon, L. M., Rauch, B. J., Walton, R. T., Berry, J. D., Joung, J. K., Kleinstiver, B. P., et al. (2018). Discovery of widespread type I and type V CRISPR-Cas inhibitors. Science 362, 240-242.
Marraffini, L. A., and Sontheimer, E. J. (2008). CRISPR interference limits horizontal gene transfer in staphylococci by targeting DNA. Science 322, 1843-1845.
Mans, C. F., and Howe, M. M. (1990). Kinetics and regulation of transcription of bacteriophage Mu. Virology 174, 192-203.
Park, C., and Zhang, J. (2012). High expression hampers horizontal gene transfer. Genome Biol Evol 4, 523-532.
Pawluk, A., Amrani, N., Zhang, Y., Garcia, B., Hidalgo-Reyes, Y., Lee, J., Edraki, A., Shah, M., Sontheimer, E. J., Maxwell, K. L., et al. (2016a). Naturally Occurring Off-Switches for CRISPR-Cas9. Cell 167, 1829-1838 e1829.
Pawluk, A., Shah, M., Mejdani, M., Calmettes, C., Moraes, T. F., Davidson, A. R., and Maxwell, K. L. (2017). Disabling a Type I-E CRISPR-Cas Nuclease with a Bacteriophage-Encoded Anti-CRISPR Protein. mBio 8.
Pawluk, A., Staals, R. H., Taylor, C., Watson, B. N., Saha, S., Fineran, P. C., Maxwell, K. L., and Davidson, A. R. (2016b). Inactivation of CRISPR-Cas systems by anti-CRISPR proteins in diverse bacterial species. Nat Microbiol 1, 16085.
Pell, L. G., Liu, A., Edmonds, L., Donaldson, L. W., Howell, P. L., and Davidson, A. R. (2009). The X-ray crystal structure of the phage lambda tail terminator protein reveals the biologically relevant hexameric ring structure and demonstrates a conserved mechanism of tail termination among diverse long-tailed phages. J Mol Biol 389, 938-951.
Piya, D., Vara, L., Russell, W. K., Young, R., and Gill, J. J. (2017). The multicomponent antirestriction system of phage P1 is linked to capsid morphogenesis. Mol Microbiol 105, 399-412.
Pursey, E., Sunderhauf, D., Gaze, W. H., Westra, E. R., and van Houte, S. (2018). CRISPR-Cas antimicrobials: Challenges and future prospects. PLoS Pathog 14, e1006990.
Quax, T. E., Voet, M., Sismeiro, 0., Dillies, M. A., Jagla, B., Coppee, J. Y., Sezonov, G., Forterre, P., van der Oost, J., Lavigne, R., et al. (2013). Massive activation of archaeal defense genes during viral infection. J Virol 87, 8419-8428.
Rauch, B. J., Silvis, M. R., Hultquist, J. F., Waters, C. S., McGregor, M. J., Krogan, N. J., and Bondy-Denomy, J. (2017). Inhibition of CRISPR-Cas9 with Bacteriophage Proteins. Cell 168, 150-158 e110.
Salis, H. M., Mirsky, E. A., and Voigt, C. A. (2009). Automated design of synthetic ribosome binding sites to control protein expression. Nat Biotechnol 27, 946-950.
Sambrook, J., and Russell, D. W. (2006). Purification of Bacteriophage lambda Particles by Isopycnic Centrifugation through CsCl Gradients. CSH Protoc 2006.
Schneider, C. A., Rasband, W. S., and Eliceiri, K. W. (2012). NIH Image to ImageJ: 25 years of image analysis. Nat Methods 9, 671-675.
Seo, S. W., Yang, J. S., Kim, I., Yang, J., Min, B. E., Kim, S., and Jung, G. Y. (2013). Predictive design of mRNA translation initiation region to control prokaryotic translation efficiency. Metab Eng 15, 67-74.
Sievers, F., Wilm, A., Dineen, D., Gibson, T. J., Karplus, K., Li, W., Lopez, R., McWilliam, H., Remmert, M., Soding, J., et al. (2011). Fast, scalable generation of high-quality protein multiple sequence alignments using Clustal Omega. Mol Syst Biol 7, 539.
Soding, J., Biegert, A., and Lupas, A. N. (2005). The HHpred interactive server for protein homology detection and structure prediction. Nucleic Acids Res 33, W244-248.
Solovyev, V., and Salamov, A. (2011). Automatic Annotation of Microbial Genomes and Metagenomic Sequences. In Metagenomics and its applications in agriculture, biomedicine, and environmental studies, R. W. Li, ed. (New York: Nova Science), pp. 61-78.
Sorek, R., Zhu, Y., Creevey, C. J., Francino, M. P., Bork, P., and Rubin, E. M. (2007). Genome-wide experimental determination of barriers to horizontal gene transfer. Science 318, 1449-1452.
Wang, P. W., Chu, L., and Guttman, D. S. (2004). Complete sequence and evolutionary genomic analysis of the Pseudomonas aeruginosa transposable bacteriophage D3112. J Bacteriol 186, 400-410.
Wang, X., Yao, D., Xu, J. G., Li, A. R., Xu, J., Fu, P., Zhou, Y., and Zhu, Y. (2016). Structural basis of Cas3 inhibition by the bacteriophage protein AcrF3. Nat Struct Mol Biol 23, 868-870.
Yosef, I., Goren, M. G., and Qimron, U. (2012). Proteins and DNA elements essential for the CRISPR adaptation process in Escherichia coli. Nucleic Acids Res 40, 5569-5576.
Young, J. C., Dill, B. D., Pan, C., Hettich, R. L., Banfield, J. F., Shah, M., Fremaux, C., Horvath, P., Barrangou, R., and Verberkmoes, N. C. (2012). Phage-induced expression of CRISPR-associated proteins is revealed by shotgun proteomics in Streptococcus thermophilus. PloS one 7, e38077.
Zhang, X., and Bremer, H. (1995). Control of the Escherichia coli rrnB P1 promoter strength by ppGpp. J Biol Chem 270, 11181-11189.

### Example 10. Critical Anti-CRISPR Locus Repression by a Bi-Functional Cas9 Inhibitor

### Summary

Bacteriophages must rapidly deploy anti-CRISPR proteins (Acrs) to inactivate the RNA-guided nucleases that enforce CRISPR-Cas adaptive immunity in their bacterial hosts. Listeria monocytogenes temperate phages encode up to three anti-Cas9 proteins, with acrIIA1 always present. AcrIIA1 inhibits Cas9 with its C-terminal domain; however, the function of its highly conserved N-terminal domain (NTD) is unknown. Here, we report that the AcrIIA1^{NTD} is a critical transcriptional repressor of the anti-CRISPR promoter. The strong anti-CRISPR promoter generates a rapid burst of transcription during phage infection and the subsequent negative feedback from AcrIIA1^{NTD} is required for optimal phage replication, even in the absence of CRISPR-Cas immunity. In the presence of CRISPR-Cas immunity, the AcrIIA1 two-domain fusion acts as a “Cas9 sensor,” tuning acr expression according to Cas9 levels. Finally, we identify AcrIIA1^{NTD} homologues in other Firmicutes, and demonstrate that they have been co-opted by hosts as “anti-anti-CRISPRs,” repressing phage anti-CRISPR deployment.

### Introduction

The constant battle for survival between bacterial predators (phages) and their hosts has led to the evolution of numerous defensive and offensive strategies in both phages and bacteria (Stern and Sorek, 2011). Bacteria employ various mechanisms to combat phages, including CRISPR-Cas adaptive immune systems that keep a record of past viral infections in a CRISPR array with phage DNA fragments (spacers) stored between repetitive DNA sequences (Mojica et al., 2005). These spacers are transcribed into CRISPR RNAs (crRNAs), which bind CRISPR-associated (Cas) proteins to guide the sequence-specific detection and nucleolytic destruction of infecting phage genomes (Brouns et al., 2008; Garneau et al., 2010).

To evade this bacterial immunity, phages have evolved many tactics, including anti-CRISPR (Acr) proteins (Borges et al., 2017). Anti-CRISPRs are highly diverse and share no protein characteristics in common; they contain distinct amino acid sequences structures (Hwang and Maxwell, 2019; Trasanidou et al., 2019). However, the anti-CRISPR genomic locus displays some recurring features, containing up to three small anti-CRISPR genes and a signature anti-CRISPR-associated (aca) gene within a single operon (Borges et at, 2017). aca genes are almost invariably present in anti-CRISPR loci and they encode repressor proteins that contain a characteristic helix-turn-helix (HTH) DNA-binding motif (Birkholz et al., 2019; Stanley et at, 2019).

Listeria monocytogenes prophages contain a unique anti-CRISPR locus without an obvious standalone aca gene. These phages do, however, encode acrIIA1, a signature anti-CRISPR gene, which contains an HTH motif in its N-terminal domain (NTD) (Rauch et al., 2017). The AcrIIA1 HTH motif is highly conserved across orthologues, yet it is completely dispensable for anti-CRISPR activity, which resides in the C-terminal domain (CTD) (companion manuscript; Osuna et al., 2020a). Thus, the role and function of the AcrIIA1NTD remains unknown. Here, we show that AcrIIA1 is a bi-functional anti-CRISPR protein that performs a crucial regulatory role as an autorepressor of acr locus transcription that is required for optimal phage fitness. AcrIIA1^{NTD} orthologues in phages and plasmids across the Firmicutes phylum also display autorepressor activity. We also show that the bacterial host can exploit the highly conserved anti-CRISPR locus repression mechanism, using the AcrIIA1^{NTD} as an “anti-anti-CRISPR” to block phage anti-CRISPR expression during phage infection and lysogeny.

### Results

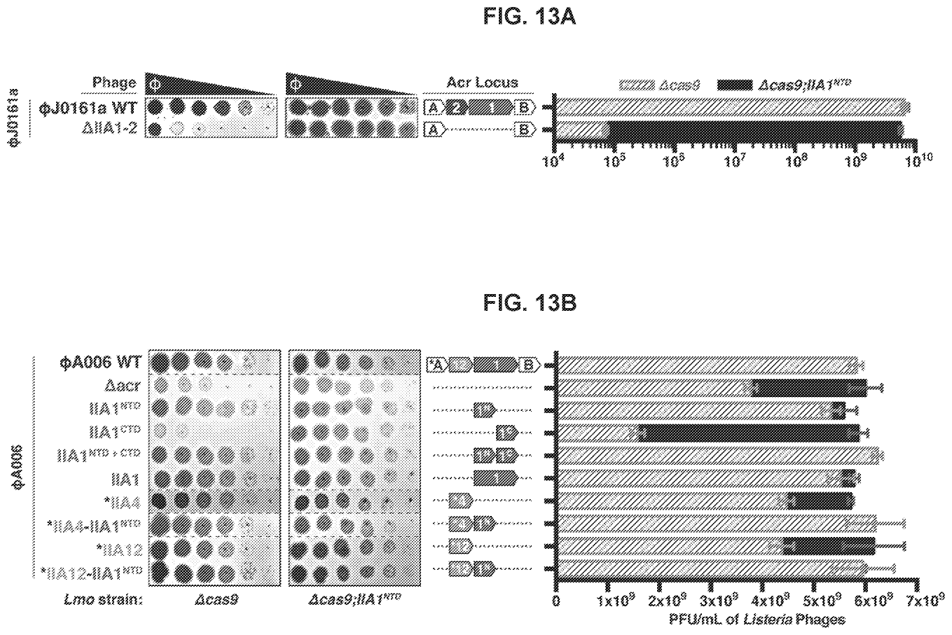

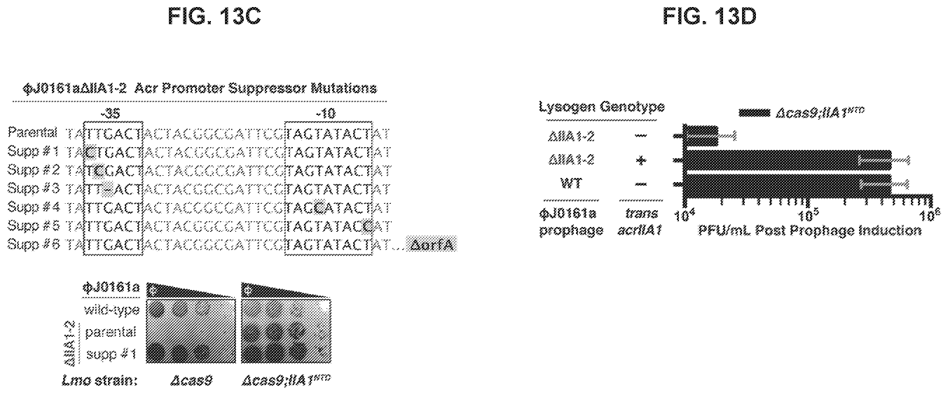

AcrIIA1^{NTD} promotes general lytic growth and prophage induction. While interrogating anti-CRISPR phages in Listeria, we observed that two phage mutants displayed a lytic replication defect when their anti-CRISPR locus was deleted (ΦJ0161aΔacrIIA1-2 and ΦA006Δacr), even in a host lacking Cas9 (FIGS. 13A and 13B). The only gene that was removed from both phages was acrIIA1, suggesting that aside from acting as an anti-CRISPR, AcrIIA1 is also generally required for optimal phage replication. AcrIIA1 is a two-domain protein with a CTD that inhibits Cas9 (Osuna et al., 2020a) and an NTD of uncharacterized function that contains a helix-turn-helix (HTH) motif similar to known transcriptional repressors (Ka et al., 2018). We hypothesized that the putative transcriptional repressor activity of AcrIIA1^{NTD} is necessary for phage replication, even in the absence of CRISPR-Cas immunity Indeed, complementation with acrIIA1^{NTD} in trans rescued the lytic growth defects of both phages containing anti-CRISPR locus deletions (FIGS. 13A and 13B). Rare spontaneous mutants (˜10 frequency) of the ΦJ0161aΔacrIIA1-2 phage that grew in the absence of acrIIA1^{NTD} complementation were isolated, revealing that mutations in the −35 and −10 promoter elements suppressed the growth defect, as did a large deletion of the region, consistent with a vital cis-acting role for AcrIIA1 (FIG. 13C).

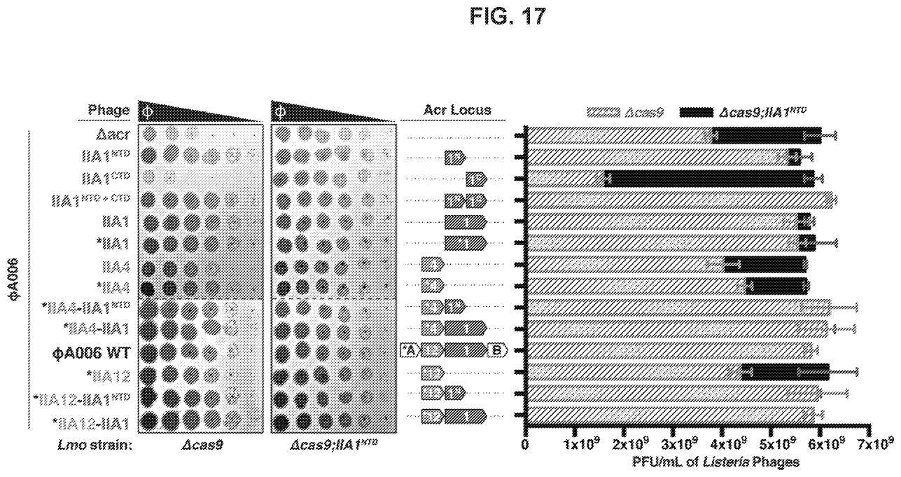

A panel of ΦA006-derived phages engineered to study anti-CRISPR deployment during phage infection (Osuna et al., 2020a) was next examined in a host lacking Cas9. The lytic growth defect was again apparent in each phage that lacked AcrIIA1 or AcrIIA1^{NTD} and providing acrIIA1^{NTD} in trans or in cis (i.e. encoded in the phage acr locus) ameliorated this growth deficiency (FIGS. 13B and 17A). The phage engineered to express acrIIA1^{CTD} alone ΦA006-IIA1^{CTD}), which is naturally always fused to acrIIA1NTD, displayed the strongest lytic defect amongst the ΦA006 phages and generated minuscule plaques (see spot titration, FIG. 13B). The plaque size and phage titer deficiencies of ΦA006-IIA1^{CTD} were fully restored with acrIIA1^{NTD} supplemented in trans and most notably, when acrIIA1^{NTD} was added to the phage genome as a separate gene ΦA006-IIA1^{NTD+CTD}, FIG. 13B). Together, these data suggest that the HTH-containing AcrIIA1^{NTD} enacts an activity that is a key determinant of phage fitness, irrespective of CRISPR-Cas immunity.

To test whether AcrIIA1^{NTD} is also important during lysogeny, prophages were induced with mitomycin C treatment and the resulting phage titer was assessed. The ΦJ0161aΔacrIIA1-2 prophage displayed a strong induction deficiency, yielding 25-fold less phage, compared to the WT prophage or the acrIIA1-complemented mutant (FIG. 13D). Attempts to efficiently induce ΦA006 prophages were unsuccessful, as previously observed (Loessner, 1991; Loessner et al., 1991). Therefore, AcrIIA1 is a bi-functional protein that not only acts as an anti-CRISPR, but also plays a critical role in the phage life cycle, promoting optimal lytic replication and lysogenic induction irrespective of CRISPR-Cas9.

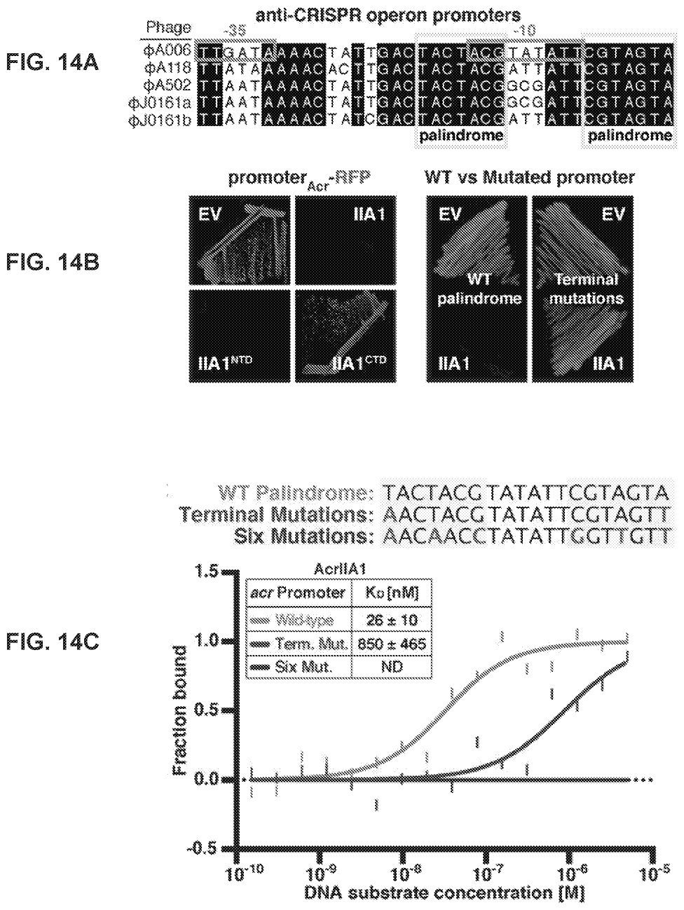

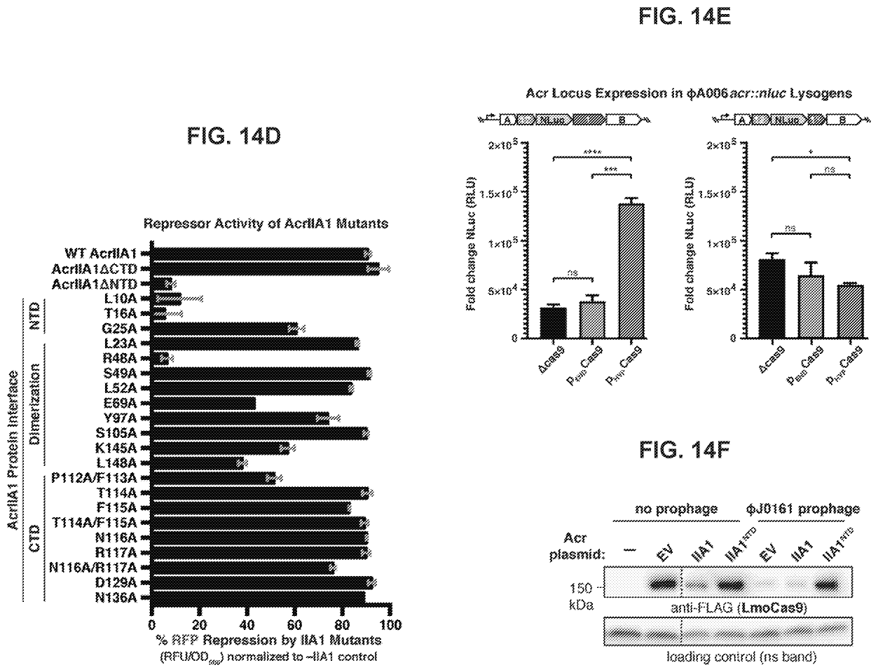

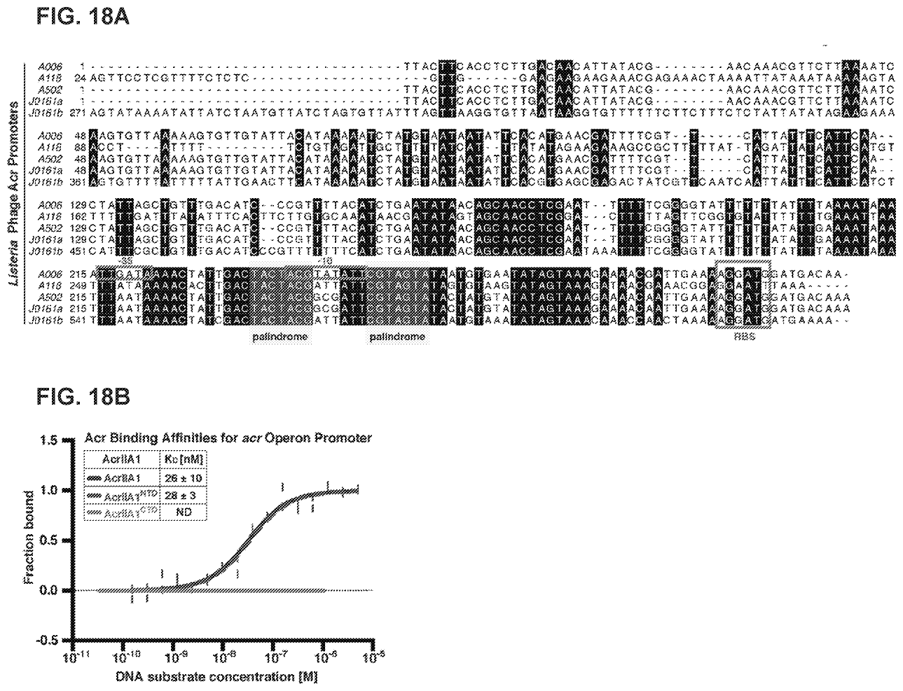

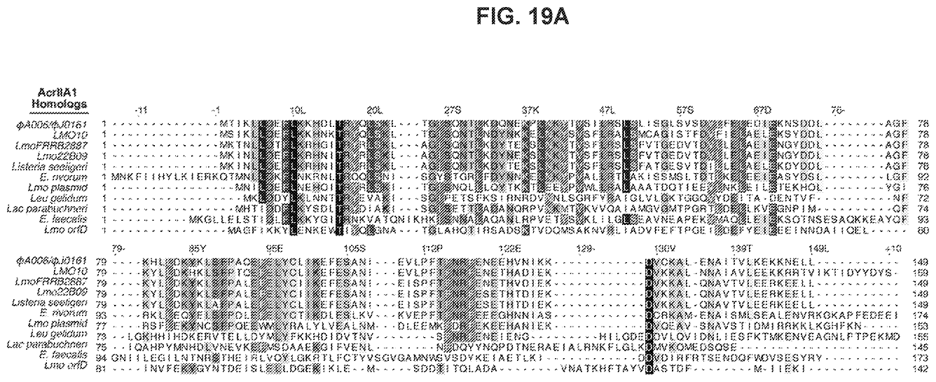

AcrIIA1^{NTD} is a repressor of the anti-CRISPR promoter and a Cas9 “sensor”. The AcrIIA1^{NTD} domain bears close structural similarity to the phage 434 cI protein (Ka et al., 2018), an autorepressor that binds specific operator sequences in its own promoter (Johnson et al., 1981). Analysis of the anti-CRISPR promoters in ΦA006, ΦJ0161, and ΦA118 revealed a conserved palindromic operator sequence (FIGS. 14A and 18A), suggesting transcriptional control by a conserved regulator such as AcrIIA1. An RFP transcriptional reporter assay showed that full-length AcrIIA1 and AcrIIA1NTD, but not AcrIIA1CTD repress the ΦA006 anti-CRISPR promoter (FIG. 14B, left panel). In vitro MST binding assays also confirmed that AcrIIA1 (K_{D}=26±10 nM) or AcrIIA1^{NTD} (K_{D}=28±3 nM), but not the AcrIIA1^{CTD}, bind the anti-CRISPR promoter with high affinity (FIGS. 14C and 18B). Moreover, mutagenesis of the terminal nucleotides of the palindromic operator sequence prevented AcrIIA1-mediated repression of the ΦA006 anti-CRISPR promoter (FIG. 14B, right panel) and abolished promoter binding in vitro (FIG. 14C). Alanine scanning mutagenesis of conserved residues predicted to be important for DNA binding and dimerization (Ka et al., 2018) identified AcrIIA1^{NTD} residues L10, T16, and R48 as critical for transcriptional repression, whereas AcrIIA1^{CTD} mutations had little effect (FIG. 14D). These data show that AcrIIA1^{NTD} represses anti-CRISPR transcription by binding a highly conserved operator, and together with the suppressors isolated above, we conclude that this repression is important due to the need to silence a strong promoter (see Discussion).

We next hypothesized that the ability of AcrIIA1 to repress transcription with one domain and inactivate Cas9 with another would enable the tuning of acr transcripts to match the levels of Cas9 in the native host, L. monocytogenes. A reporter lysogen was engineered by inserting a nanoluciferase (nluc) gene in the acr locus. Low acr expression was seen in the absence of Cas9, or during low levels of Cas9 expression, however acr reporter levels increased by ˜5-fold when Cas9 was overexpressed (FIG. 14E, left). acr induction was not seen in the absence of AcrIIA1^{CTD} (FIG. 14E, right), the Cas9 binding-domain, supporting a model where Cas9 “sensing” de-represses the acr promoter. After confirming de-repression through an increase in Cas9 levels, we sought to confirm that AcrIIA1^{NTD} is also capable of further repressing lysogenic anti-CRISPR expression. We therefore expressed the AcrIIA1^{NTD} repressor in trans and assessed anti-CRISPR function. The Cas9 degradation normally induced by prophage-expressed AcrIIA1 activity (companion manuscript; Osuna et al., 2020a) was successfully prevented by AcrIIA1^{NTD} (FIG. 14F). These data collectively demonstrate that AcrIIA1 autoregulates acr transcript levels in L. monocytogenes and can increase acr expression in response to increased Cas9 expression.

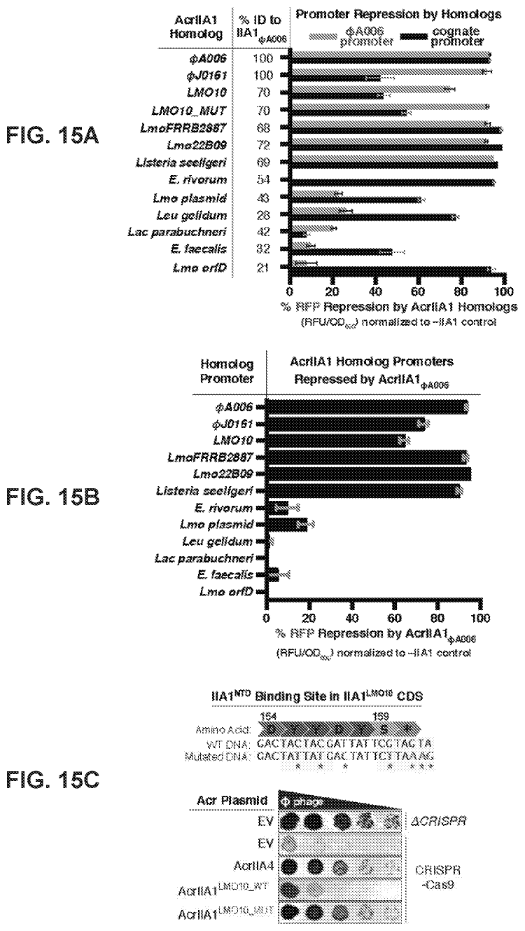

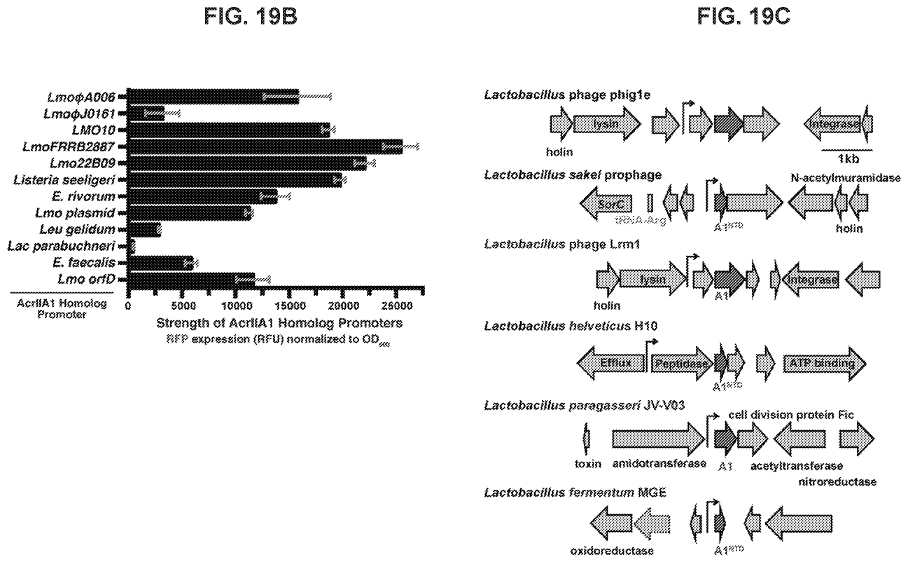

Transcriptional autoregulation is a general feature of the AcrIIA1 superfamily. Recent studies have reported transcriptional autoregulation of anti-CRISPR loci by HTH-proteins in mobile genetic elements of Gram-negative Proteobacteria (Birkholz et al., 2019; Stanley et al., 2019). To determine whether anti-CRISPR locus regulation is similarly pervasive amongst mobile genetic elements in the Gram-positive Firmicutes phylum, we assessed AcrIIA1 homologs for transcriptional repression of their predicted cognate promoters and our model ΦA006 phage promoter. Homologs sharing 21% (i.e. Lmo orfD) to 72% amino acid sequence identity with AcrIIA1^{NTD} were selected from mobile elements in Listeria, Enterococcus, Leuconostoc, and Lactobacillus (FIGS. 15A and 19A). All AcrIIA1 homologs repressed transcription of their cognate promoters by 42-99%, except AcrIIA1 from Lactobacillus parabuchneri, where promoter expression was undetectable (FIGS. 15A and 19B). Strong repression of the model ΦA006 promoter was only enacted by Listeria orthologues possessing >68% protein sequence identity (FIG. 15A). Likewise, AcrIIA1_{ΦA006} only repressed the promoters associated with orthologues that repressed the ΦA006 promoter (FIG. 15B). Interestingly, an AcrIIA1^{NTD} palindromic binding site resides in the protein-coding sequence of the AcrIIA1_{LO10} homolog, which displayed no anti-CRISPR activity despite possessing 85% AcrIIA1^{CTD} sequence identity (FIGS. 15C and 19A). When this AcrIIA1^{NTD} binding site was disrupted with silent mutations, AcrIIA1_{LMO10} anti-CRISPR function manifested (FIG. 15C), confirming that intragenic anti-CRISPR repression can also occur. Altogether, these findings demonstrate that the anti-CRISPR promoter-AcrIIA1NTD repressor relationship is highly conserved and likely performs a vital repressive function in these diverse mobile genetic elements.

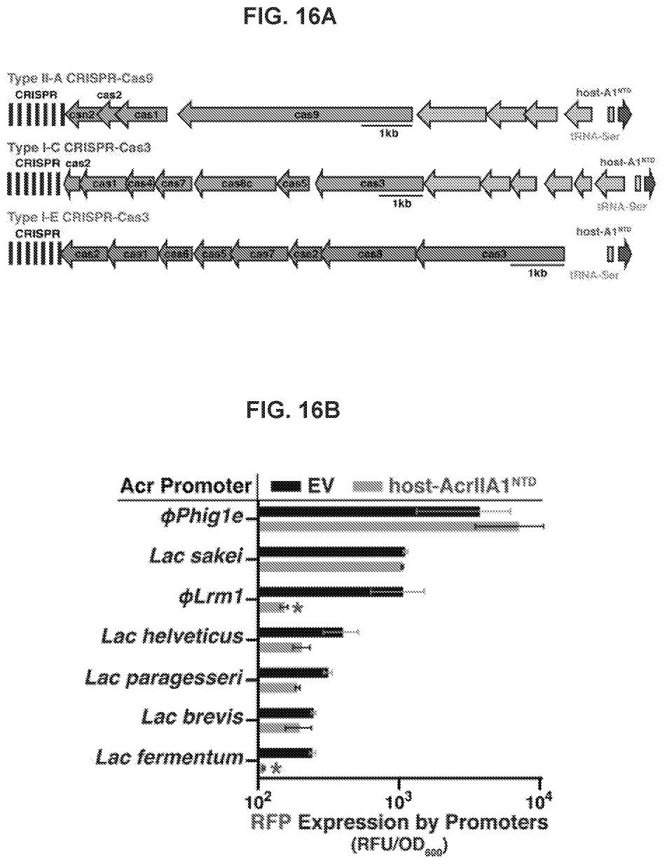

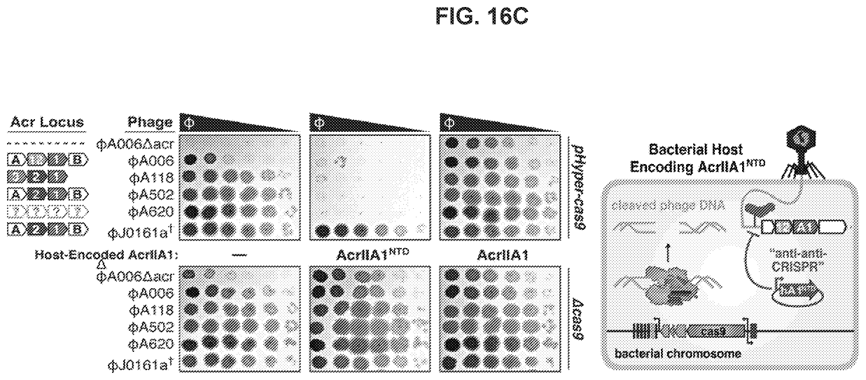

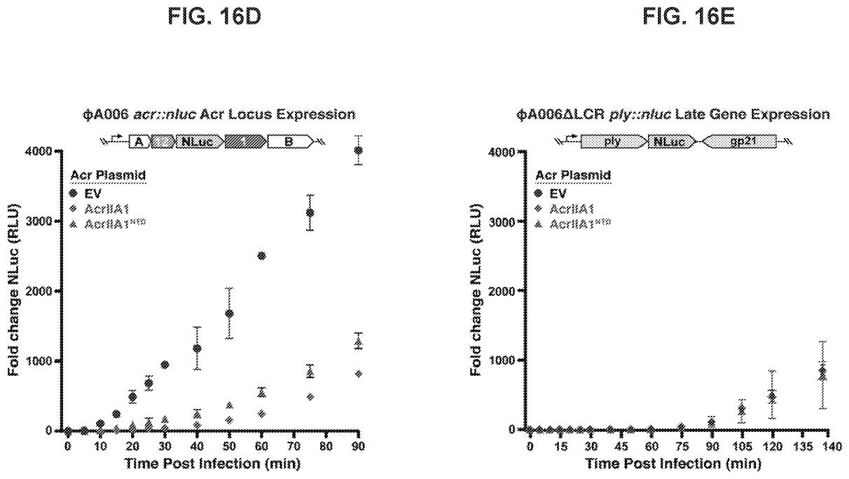

Host-encoded AcrIIA1^{NTD} blocks phage anti-CRISPR deployment. AcrIIA1^{NTD} orthologues are encoded by many Firmicutes including Enterococcus, Bacillus, Clostridium, and Streptococcus (Rauch et al., 2017). In most cases, AcrIIA1^{NTD} is fused to distinct AcrIIA1^{CTDs} in mobile genetic elements, which are likely anti-CRISPRs that inhibit CRISPR-Cas systems in their respective hosts. Interestingly, there are instances where core bacterial genomes encode AcrIIA1^{NTD} orthologues that are short ˜70-80 amino acid proteins possessing only the HTH domain. One example is in Lactobacillus delbrueckii, where strains contain an AcrIIA1^{NTD} homolog (35% identical, 62% similar to AcrIIA1_{ΦA006}) with key residues conserved (e.g., L10 and T16). Given that AcrIIA1^{NTD} represses anti-CRISPR transcription, we wondered whether bacteria could co-opt this regulator and exploit its activity in trans, preventing a phage from deploying its anti-CRISPR arsenal. Remarkably, we observed that the L. delbrueckii AcrIIA1^{NTD} homolog is always a genomic neighbor of either the Type I-E, I-C, or II-A CRISPR-Cas systems in this species (FIG. 16A), and these CRISPR-associated AcrIIA1^{NTD} proteins are highly conserved (>95% sequence identity). This association is supportive of an “anti-anti-CRISPR” role that aids CRISPR-Cas function by repressing the deployment of phage inhibitors against each system. Although there are no specific anti-CRISPR proteins identified in Lactobacillus phages and prophages that express anti-CRISPRs, we reasoned that phages with their own acrIIA1 homolog might have acr loci that would be vulnerable to repression by the host protein. Fluorescent reporters were built, driven by seven different Lactobacillus phage or prophage promoters that possess an acrIIA1 homolog in their downstream operon (FIG. 19C). This enabled the identification of one promoter, from phage Lrm1, that was robustly repressed by L. delbrueckii host AcrIIA1NTD. This confirms that a bona fide acr locus in a Lactobacillus phage can be repressed by a host version of a hijacked acr repressor (FIG. 16B).

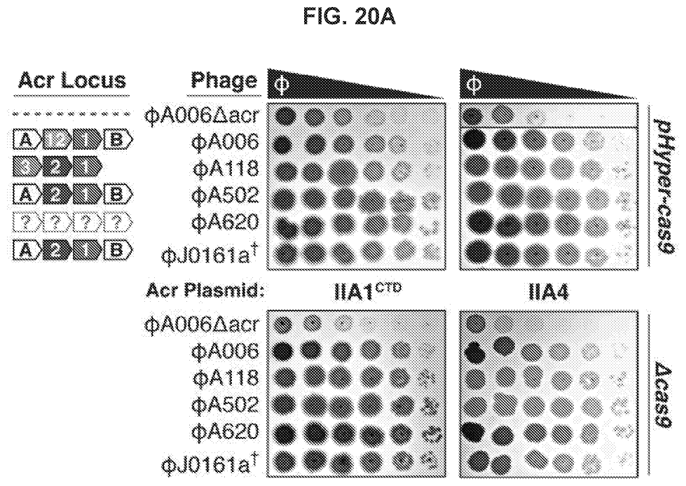

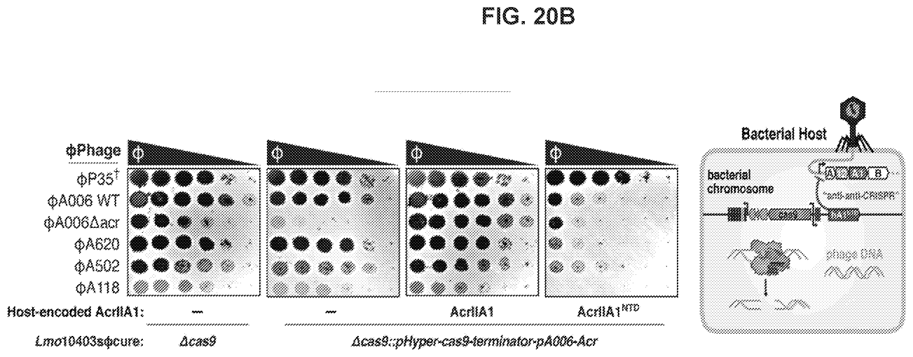

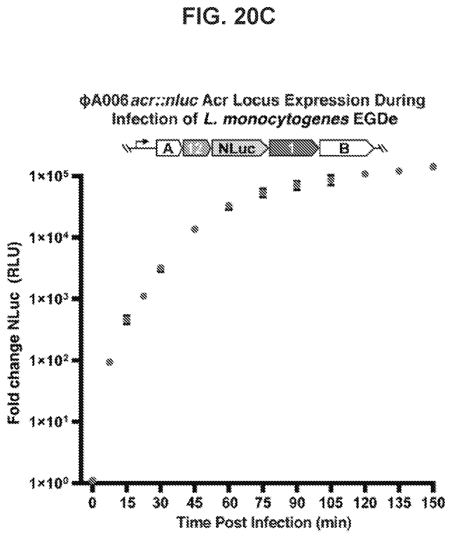

To interrogate the anti-anti-CRISPR prediction in a native phage assay, we expressed AcrIIA1^{NTD} from a plasmid (FIGS. 16B and 20B) or from an integrated single-copy acrIIA1^{NTD} driven by its cognate phage promoter (FIG. 20B) in L. monocytogenes. A panel of distinct anti-CRISPR-encoding phages became vulnerable to Cas9 targeting when AcrIIA1^{NTD} was expressed by the host (FIGS. 16C and 20B), whereas expression of full-length AcrIIA1, AcrIIA1^{CTD}, or AcrIIA4 had the expected anti-CRISPR phenotype (FIGS. 16C and 20A). Each of these phages possesses complete or partial spacer matches to the Lmo10403s CRISPR array. In contrast, replication of the non-targeted phages, ΦJ0161a (FIG. 16C) and ΦP35 (FIG. 20B), was unperturbed. Additionally, the acr::nluc reporter phage was used in a similar experiment, confirming that acr expression rapidly occurs during infection and can be silenced by expression of AcrIIA1 or AcrIIA1^{NTD} (FIG. 16D), while a model late promoter (ply::nluc) was not silenced (FIG. 16E). These data demonstrate that hosts can use the anti-CRISPR repressor to block anti-CRISPR synthesis, rendering a phage unable to express its Acr proteins.

### Discussion

The Listeria phage anti-CRISPR AcrIIA1 was first described as a Cas9 inhibitor, and here we demonstrate that it is also a transcriptional autorepressor of the acr locus required for optimal lytic growth and prophage induction. Notably, this bi-functional regulatory anti-CRISPR has the ability to tune acr transcription in accordance with Cas9 levels.

Transcriptional autorepression is seemingly the predominant regulatory mechanism in bacteria and phages, as 40% of transcription factors in E. coli exert autogenous negative control (Thieffry et al., 1998). Due to their short response times, negative autoregulatory circuits are thought to be particularly advantageous in dynamic environments where rapid responses improve fitness. A strong promoter initially produces a rapid rise in transcript levels and after some time, repressor concentration reaches a threshold, shutting off its promoter to maintain steady-state protein levels (Madar et al., 2011; Rosenfeld et al., 2002). During infection, phages must rapidly produce anti-CRISPR proteins to neutralize the preexisting CRISPR-Cas complexes in their bacterial host. Consistent with the rapid response times exhibited by negatively autoregulated promoters, we observed a burst of anti-CRISPR locus expression within ten minutes post infection using a reporter phage (FIGS. 16C and 20C). During lysogeny, autorepression by AcrIIA1 presumably tempers anti-CRISPR locus expression, generating steady-state anti-CRISPR levels to maintain Cas9 inactivation.

Negative autoregulation maintains precise levels of the proteins encoded by the operon to prevent toxic effects caused by their overexpression (Thieffry et al., 1998), as classically observed with the λ phage genes cII and N (Shimatake and Rosenberg, 1981). In this study, the engineered ΦA006-IIA1^{CTD} phage, which only contains the AcrIIA1^{CTD} and lacks the AcrIIA1^{NTD} autorepressor, displayed a pronounced lytic growth defect, even stronger than the defect of the ΦA006^{Δacr} phage that completely lacks anti-CRISPRs (FIG. 13B). This suggests that the AcrIIA1^{NTD} autoregulatory domain is fused to AcrIIA1^{CTD in nature to limit the expression of an anti-CRISPR domain that can be toxic to the phage. Phages expressing only AcrIIA}4 or AcrIIA12 were only mildly affected by the absence of AcrIIA1^{NTD} (FIG. 13B). However, other Listeria phage anti-CRISPRs (such as AcrIIA3) have been shown to exert toxic effects (Rauch et al., 2017), underscoring the need for an autoregulatory mechanism that tempers anti-CRISPR levels. The ΦJ0161a phage displays a remarkably strong growth defect when AcrIIA1 is absent (ΦJ0161aΔacrIIA1-2, FIG. 13A), which is suppressed by promoter mutations or deletion of orfA (FIG. 13C), suggesting that misregulation of a gene within the acr locus may be deleterious. Constitutively strong promoter activity may also have other deleterious effects. A recent study demonstrated that neighboring phage genes can be temporally misregulated in the absence of an anti-CRISPR locus autorepressor, Aca1 (Stanley et al., 2019).

Beyond cis regulatory auto-repression, prophages may also use AcrIIA1^{NTD} to combat phage superinfection, benefitting both the prophage and host cell. The phage lambda cI protein, for example, represses prophage lytic genes and prevents superinfection by related phages during lysogeny (Johnson et al., 1981). Similarly, a lysogen could use AcrIIA1^{NTD} to bolster the activity of a second CRISPR-Cas system in its host (such as the Type I-B system that is common in Listeria) by preventing incoming phages from expressing their Type I-B anti-CRISPRs. Host expressed AcrIIA1^{NTD} does manifest as an anti-anti-CRISPR, blocking anti-CRISPR expression from infecting or integrated phages (FIGS. 16B and 20B). We also demonstrate that AcrIIA1^{NTD} orthologues that reside in non-mobile regions of bacterial genomes can perform as a bona fide anti-CRISPR repressor. Thus, the importance of the conserved anti-CRISPR locus repression mechanism may represent a weakness in the phage, which can be exploited by the host through the co-opting of this anti-CRISPR regulator.

### Example 11. Inhibition of AcrIC1 by aca1

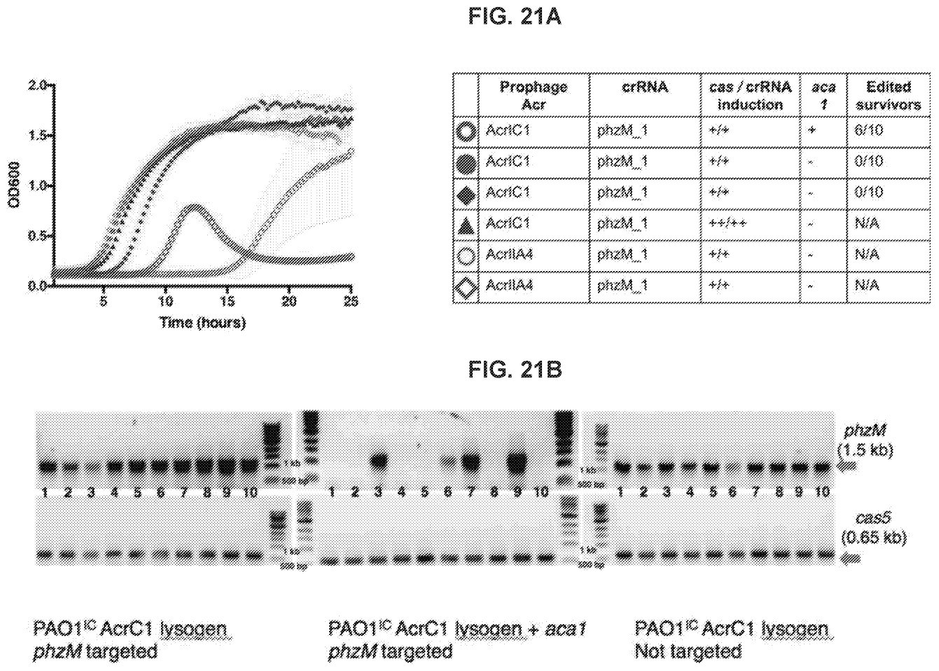

One potential impediment to the implementation of any CRISPR-Cas bacterial genome editing tool is the presence of anti-CRISPR (acr) proteins that inactivate CRISPR-Cas activity. In the presence of a prophage expressing AcrIC1 (a Type I-C anti-CRISPR protein) from a native acr promoter, self-targeting was completely inhibited, but not by an isogenic prophage expressing a Cas9 inhibitor AcrIIA435 (FIG. 21A). To attempt to overcome this impediment, we expressed aca1 (anti-CRISPR associated gene 1), a direct negative regulator of acr promoters, from the same construct as the crRNA. Using this repression-based “anti-anti-CRISPR” strategy, CRISPR-Cas function was re-activated, allowing the isolation of edited cells despite the presence of acrIC1 (FIGS. 21A and 21B). In contrast, simply increasing cas gene and crRNA expression did not overcome AcrIC1-mediated inhibition (FIG. 21A). Therefore, using anti-anti-CRISPRs presents a viable route towards enhanced efficiency of CRISPR-Cas editing and necessitates continued discovery and characterization of anti-CRISPR proteins and their cognate repressors.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of activating CRISPR-Cas to target a nucleic acid in a bacterial cell expressing an anti-CRISPR (Acr) protein, the method comprising:
introducing an anti-CRISPR-associated (Aca) protein into the bacterial cell, wherein the Aca protein represses expression of the Acr protein, thereby allowing the Cas protein to target the nucleic acid as directed by a guide RNA.

2. The method of claim 1, further comprising introducing the guide RNA into the bacterial cell.

3. The method of claim 1 or 2, wherein the Cas protein is endogenous to the bacterial cell.

4. The method of claim 1 or 2, wherein the Cas protein is exogenous to the bacterial cell.

5. The method of claim 4, wherein the method further comprises introducing the Cas protein into the bacterial cell.

6. The method of any one of claims 1 to 5, wherein the Cas protein is selected from the group consisting of Cas3, Cas5, Cas6, Cas7, Cas8, Cas9, Cas10, Cas12, and Cas13.

7. The method of claim 6, wherein the Cas protein is Cas3, Cas9, or Cas12.

8. The method of any one of claims 1 to 7, wherein the introducing step comprises introducing a polynucleotide encoding the Aca protein into the bacterial cell, and wherein the Aca protein is expressed in the bacterial cell.

9. The method of claim 8, wherein the introducing step comprises contacting the bacterial cell with a phage that encodes the Aca protein, wherein the phage introduces a polynucleotide encoding the Aca protein into the bacterial cell and the bacterial cell expresses the Aca protein.

10. The method of claim 8, wherein the introducing step comprises contacting the bacterial cell with a conjugation partner bacterium comprising a polynucleotide that encodes the Aca protein, wherein the Aca protein or a polynucleotide encoding the Aca protein is introduced from the conjugation partner bacterium to the bacterial cell by bacterial conjugation.

11. The method of any one of claims 1-10, wherein the method occurs within a mammalian host of the bacterial cell.

12. The method of claim 11, wherein the bacterial cell resides in the gut of the mammalian host.

13. The method of claim 11 or 12, wherein the mammalian host is a human.

14. The method of any one of claims 1 to 13, wherein the nucleic acid is DNA.

15. The method of any one of claims 1 to 13, wherein the nucleic acid is RNA.

16. The method of claim 14, wherein the DNA is present within the bacterial chromosome.

17. The method of any of claims 1-16, wherein the Aca protein is substantially (at least 60%, 70%, 80%, 90%, 95%) identical to any one of SEQ ID NOS: 1-27 or SEQ ID NOS: 50-60.

18. A polynucleotide comprising a promoter operably linked to a sequence encoding an Aca protein substantially (at least 60%, 70%, 80%, 90%, 95% identical) to any one of SEQ ID NOS: 1-27 or SEQ ID NOS: 50-60, wherein the promoter is heterologous to the sequence.

19. The polynucleotide of claim 18, wherein the promoter is a constitutive promoter.

20. A plasmid comprising the polynucleotide of claim 18 or 19.

21. A phage comprising a polynucleotide encoding an anti-CRISPR associated (Aca) protein, wherein the polynucleotide is heterologous to the phage.

22. The phage of claim 21, further comprising a polynucleotide encoding a guide RNA.

23. The phage of claim 21 or 22, further comprising a polynucleotide encoding a Cas protein.

24. The phage of claim 23, wherein the Cas protein is selected from the group consisting of Cas3, Cas5, Cas6, Cas7, Cas8, Cas9, Cas10, Cas12, and Cas13.

25. The phage of claim 24, wherein the Cas protein is Cas3, Cas9, or Cas12.

26. The phage of any one of claims 21 to 25, wherein the Aca protein is substantially (at least 60%, 70%, 80%, 90%, 95%) identical to any one of SEQ ID NOS: 1-27 or SEQ ID NOS: 50-60.

27. A bacterial cell comprising a polynucleotide encoding an anti-CRISPR associated (Aca) protein, wherein the polynucleotide is heterologous to the cell.

28. The bacterial cell of claim 27, wherein the bacterial cell is from a species selected from the group consisting of Pseudomonas aeruginosa, Pseudomonas otitidis, Pseudomonas delhiensis, Vibrio parahaemolyticus, Shewanella xiamenensis, Brackiella oedipodis, Oceanimonas smirnovii, Neisseria meningitides, Pseudomonas stutzeri, Yersinia frederiksenii, Escherichia coli, Serratia fonticola, Dickeya solani, Pectobacterium carotovorum, Enterobacter cloacae, Alcanivorax sp., Halomonas caseinilytica, Halomonas sinaiensis, Cryptobacterium curtum, Pseudomonas sp., Corynebacterium sp., Bacillus subtitis, Streptococcus pneumonia, Staphylococcus aureus, Campylobacter jejuni, Francisella novicida, Corynebacterium diphtheria, Enterococcus sp., Listeria monocytogenes, Mycoplasma gallisepticum, Streptococcus sp., and Treponema denticol.

29. The bacterial cell of claim 27 or 28, further comprising a polynucleotide encoding a guide RNA.

30. The bacterial cell of any one of claims 27 to 29, further comprising a polynucleotide encoding a Cas protein.

31. The bacterial cell of any one of claims 27 to 30, wherein the Aca protein is substantially (at least 60%, 70%, 80%, 90%, 95%) identical to any one of SEQ ID NOS: 1-27 or SEQ ID NOS: 50-60.


SEQUENCE LISTING (230 sequences)

SEQ ID NO: 1 - protein, 79 aa, Pseudomonas aeruginosa
MRFPGVKTPDASNHDPDPRYLRGLLKKAGISQRRAAELLGLSDRVMRYYLSEDIKEGYRPAPYTVQFALECLANDPPSA

SEQ ID NO: 2 - protein, 82 aa, Pseudomonas aeruginosa
MQLKPRNTVPRPDASSHNPDPRYLRGLLKKAGISQRRAAELLGLGDRVMRYYLSEDAKDGYRPAPYTVQFALECLANDPPSA

SEQ ID NO: 3 - protein, 71 aa, Pseudomonas otitidis
MKPDASNHNPDPRYLRELIERAGVSQRQAAELIGMSWEGFRRYLRDVDAPGYRVADYRVQFALECLAAPGT

SEQ ID NO: 4 - protein, 79 aa, Pseudomonas delhiensis
MPLQQRSTVRKPDASNHNPNPRYLRGLVERSGKSQRQAAELLGLSWEGFRNYLRDESHPLHRSAPYTVQFALECLAEAE

SEQ ID NO: 5 - protein, 67 aa, Pseudomonas aeruginosa
MKPDSSKHNPDPQYLRGLYERAGLKQEEAARRIGITARALRNYVSETAGREAPYPVQFALECLASES

SEQ ID NO: 6 - protein, 65 aa, Pectobacterium phage ZF40
MTAMKEWRARMGWSQRRAAQELGVTLPTYQSWEKGIRLSDGSPIDPPLTALLAAAAREKGLPPIS

SEQ ID NO: 7 - protein, 128 aa, Vibrio parahaemolyticus
MPLLFRSFIMTNQELKQLRRLLFIEVSEAAALIGECEPRTWQRWEKGDRAIPNDVSREIQMLALTRLERLQVEFDETDPNYRYFETFDEYKAYGGTGNELKWRLAQSVATSLLCETEADKWREEETID

SEQ ID NO: 8 - protein, 120 aa, Shewanella xiamenensis
MTNTELKQLRTLLFLDVTEAAQHIGDCEPRTWQRWEKGDRAVPVDVAQTMQMLALTRVDMLQVEYDAADPMYQYFSEYEDFKAATGATGASVLKWRLAQSVSAQLVSEQQAEIWRAEETI

SEQ ID NO: 9 - protein, 151 aa, Brackiella oedipodis
MNGQELKKARALLNLSQQEAAKLIGDVSKRSWVFWESGRPSIPQDVQEKFNDLLMRRKAIVQPFIDKTISPSNVYRIYLDQNDLAFISDPIELRLLQGVALTLHFDYDLPLVDFDMKDYEQWLQDQDKTDDPTTRSEWASTNHPCSSKISD

SEQ ID NO: 10 - protein, 125 aa, Oceanimonas smirnovii
MTHYELQALRKLLMLEVSEAAREIGDVSPRSWQYWESGRSPVPDDVANQIRNLTDMRYQLLELRTEQIEKAGKPIQLNFYRTLDDYEAVTGKRDVVSWRLTQAVAATLFAEGDVTLVEQGGLTLE

SEQ ID NO: 11 - protein, 79 aa, Neisseria meningitidis
MKMRRIWRAGMIDNPELGYTPANLKAIRQKYGLTQKQVADITGATLSTAQKWEAAMSLKTHSDMPHTRWLLLLEYVRNL

SEQ ID NO: 12 - protein, 72 aa, Pseudomonas aeruginosa
MTPDQFDALAELIRLRGGASQEAARLVLVDGMSPSDAARQVEASPQAVSNVLASCRRGLALVLRASGKGATA

SEQ ID NO: 13 - protein, 67 aa, Pseudomonas aeruginosa
MTKEQFSALAELMRLRGGPGQDAARLVLVNGLKPTEAARQTGITPQAVNKTLSSCRRGIELAKRVFT

SEQ ID NO: 14 - protein, 68 aa, Pseudomonas stutzeri
MMTGEQFGALAELLRLRGGASQEAARLVLVEGLAPAEAARQAGTTPQAVSNALASCRRGLELARVAAG

SEQ ID NO: 15 - protein, 68 aa, Pseudomonas sp.
MTAEQFSALAELLRLRGGASQEAARLVLVEQLTPAEAARAAGCSPQAVSNVLASCRRGLELAHAAVGH

SEQ ID NO: 16 - protein, 64 aa, Yersinia frederiksenii
MPLIEYIRLTFSGNKSEFARHMGVDRQKVQVWIKGEWIVVGNKLYAPRRDIPDIRLDTVSQRLD

SEQ ID NO: 17 - protein, 68 aa, Escherichia coli
MNKMNARTLSDYIAFYHNGNQAEFARHMGVNRQQVTKWIKGGWIVINHQLFSPQRDIPENISHGGSAL

SEQ ID NO: 18 - protein, 72 aa, Serratia fonticola
MNNDNLVSGRTLLGYINIFHNGSQADFARHMDVTPQQVTKWISGEWIVVNHQLFSPKRDVPENISGGESAGN

SEQ ID NO: 19 - protein, 66 aa, Dickeya solani
MKLSEFIDTEFSGSRAEFARLMGVRPQKVNDWLVAGMIIHIDENGQAFLCSVRRDIPAWNRKTNFA

SEQ ID NO: 20 - protein, 60 aa, Pectobacterium carotovorum
MSLTEYIDKNFGGNKAAFARHMGVDAQAVNKWIKSEWFVSTTDDNKIYLSSARREIPPLK

SEQ ID NO: 21 - protein, 65 aa, Enterobacter cloacae complex
MNARTLSDYIEFYHNGNQSDFARHMGVNRQQVTKWLNGGWVVINHQLYSPQRDVPEFVTGGGSAL

SEQ ID NO: 22 - protein, 60 aa, Pectobacterium carotovorum
MSLTEYIDKNFAGNKAAFARHMGVDAQAVNKWIKSEWFVSTTDDNKIYLSSVRREIPPVA

SEQ ID NO: 23 - protein, 65 aa, Alcanivorax sp.; identical to SEQ ID NO: 6

SEQ ID NO: 24 - protein, 64 aa, Alcanivorax sp.
MTAMKDWRTRMGWSQRRAAQELGVTLPTYQSWERGVRLSDGSLIDPPLTALLAAAAREKGLDPI

SEQ ID NO: 25 - protein, 68 aa, Halomonas caseinilytica
MIDARKHYDPNLAPELVRRALAVTGTQKELAERLDVSRTYLQLLGKGQKSMSYAVQVMLEQVIQDGET

SEQ ID NO: 26 - protein, 68 aa, Halomonas sinaiensis
MIDARKYYNPDLAPELVSRALAVTGTQKELAERLDVSRIYIQLLGKGQKTMSYAVQVMLEQVIQGGEN

SEQ ID NO: 27 - protein, 196 aa, Cryptobacterium curtum
MPIKDLTGMRFGRLVVKEATSRRTSDGNVIWRCQCDCGNVTEVPGHSLTRGNTRSCGCGEEENRRESGNNRNKAVVKEHSRADSFLSPKPRADTTLGIRGILRRPSGRYAARITFKGKTTCLGTYDSLEEAANARREAEIEIFDPYLIANGLPPTSEEEWQKILARALEKEKDNADTSTKARPGKIRARKNKAVQN

SEQ ID NO: 28 - DNA, 125 bp, Unknown
  Description of Unknown promoter sequence
gggcgttaggggaaatgaattcggacaagcggcacattgtgcctattgcgtattaggcacaatgtgcctaatctagcgtcatgccagccacaacggcgaggcgaacccaaggagagacaccatga

SEQ ID NO: 29 - DNA, 125 bp, Unknown
  Description of Unknown promoter sequence
gggcgttaggggaaatgaattcggacaagcggcacattgtgcctattgcggattaggcacaatgtgcctaatctagcgtcatgccagccacaacggcgaggcgaacccaaggagagacaccatga

SEQ ID NO: 30 - DNA, 125 bp, Unknown
  Description of Unknown promoter sequence
tggcgctagggggaatgaattcggacaagcggcacattgtgcctattgcgtattaggcacaatgtgcctaatctagcctcatgccagccacaacggcgaggcgctaacaaggatcgaagctatga

SEQ ID NO: 31 - DNA, 123 bp, Unknown
  Description of Unknown promoter sequence
aatcggtagtggccactttcggacaagcggcacactgtgcctattgcgaattaggcacaatgtgcctaatctaacgtcatgccagccacaacggcgaggcgccaacaaggattcaaaccatga

SEQ ID NO: 32 - DNA, 123 bp, Unknown
  Description of Unknown promoter sequence
aatcggtagtggccactttcggacaagcggcacattgtgcctattgcgaattaggcacaatgtgcctaatctaacgtcatgccagccacaacggcgaggcgccaagaaggatagaagccatga

SEQ ID NO: 33 - DNA, 124 bp, Unknown
  Description of Unknown promoter sequence
aatcggtagtgggccactttcggacaagcggcacattgtgcctattgcgtattaggcacaatgtgcctaatctaacgtcatgccaaccacaacggcgaggcgcaaacaaggatagacaccatga

SEQ ID NO: 34 - DNA, 125 bp, Unknown
  Description of Unknown promoter sequence
ggtcgctagagggaattcattcggataagcggcacattgtgcctattgtgcattaggcacaatgtgcctaatatggcgtcatgccagccacaatggcgaggcgccacgaaggaacgatgccatgg

SEQ ID NO: 35 - DNA, 274 bp, Phaseolibacter flectens
tggttatcacccttcaaaaaagagacctccgctcactagaacgcccacacccgacttcaccatgcagtggtgtcctcggaggtcgctttcgtgaaaagtagtctcgggatttaatttaacgcagtgagtgcgatttattgcagatgcaaaaaagcccgcattaagcgagcataaaattaattaaaaaaagtattgactccggtcgcgtttgcgaccataatgtacttactgactaggcaaggggtctggtcaactcaaatagtgagattaaaaa

SEQ ID NO: 36 - DNA, 273 bp, Proteus penneri
tgcgcatatacaccccctacggagtgtccgagtttagttaaaaggatgcagacacacagctcttgtgtgaagtgattagtgtgtgattgatactgtggtctgcatatacgaaaaaagaccgcctaagcgatcttctgaatgtgattcaagtcaaaattttaagttatgtatattaatttcataatatcgcttgcctttggtcgctattgcgaccatgctgtattcatcgggtaggcaataaggcagacccaactcaactaagtgagaatatta

SEQ ID NO: 37 - DNA, 92 bp, Shewanella xiamenensis
ataaaacacttgcaatcggtcgcaatcgcgaccataatatatttaacggttcgggagtggctcgaatcgactcaataaagtgagaaatatca

SEQ ID NO: 38 - DNA, 280 bp, Vibrio parahaemolyticus
agcatccaccttccccagtcatgttcacatgataagatgaagaaaaaataggcgccctcaacttgacggcgcctatgatggacagctcaacgtattttgacttttggtggcagtattgagcgattgacgttgttagtttttaaggatattcggaaaagagtgtgtatcgggtaagttaaaataatatcaaattaacacttgcaaccggtcgcaattgcgaccataatgtattcaacggttcggaactggttcgaatcgactcaagaaagtgagatacata

SEQ ID NO: 39 - DNA, 214 bp, Vibrio cyclitrophicus
tcttgaaacctgttacataattcatagttttgattagtgtaacggtaatcaaaactcgtcacaagatatacaaaacgggtattcggaaaagagtgtgtatcggataagttaaaataaattcaaattaacacttgcaaatggtcgcaattgcgaccataatatcttcaacggttcggaactggttcgaatcgactcaagaaagtgagatacatca

SEQ ID NO: 40 - DNA, 140 bp, Pectobacterium phage ZF40
agcctcacctccggcgttgccgtggcgctgtgtgatttacaggaaataaaaaggccacgaatgcggccttagcgattaaaaaatatgaaatgccttgcttgttcgcgattgcgaacatataatttattcatcggttcgag

SEQ ID NO: 41 - DNA, 156 bp, Oceanimonas smirnovii
tgatgtgtctctctttagaggtgattcgagccaatctcgaatctggttccatcttagttcgcaattgcgaacagtgcaagagataaagtaaagaaaatacaaaaatcagccaatctgcttcctgggggttaacggtgaagcgtgggggcgagcttt

SEQ ID NO: 42 - DNA, 247 bp, Brackiella oedipodis
aatccaaacgtattaatttgttgttaaaatccaataaaaaacattacaaaagtattgacaataaatattgcataagtaataatcaaaccatagaatcacttcttgctctttaacaatcaactgaatagtcagtcagtcaactgatagaaacctactcccaatggaatagacttatggggttcaacggtcgggcagcccccaaacagaatagccgtgcgtggtgaaagcgcagagccgattaatttcc

SEQ ID NO: 43 - DNA, 356 bp, Neisseria meningitides
aattgaatccgcaatggtgaaatatcgacaatgaacgacaacacgcaaacccttcccccgcgccacctgtccgtcgccccgatgctcgactgtatctactgaaaaataatatattgaaaaataatatataatatatttttattattcttatggtgcaaataaagcacattgtgcattggaaataaaaacggcaaattaattacctttgtttttaaaggtttattaaattggcggttttttgtttgaaaaaatgcttgttatacatcattttttgatgtagtatacacacatcggcagacaacaagcctgccaccgacaccttgacggtttcaaggataaacgaaaggatttcaaaa

SEQ ID NO: 44 - DNA, 355 bp, Neisseria meningitides
aattgaatccgcaatgatgaaatatcgacaatgaacgacaatacacacacccttcccccgcgccgcctttccgtcgccccgatgctcgactgtatctactgaaaaataatatattgaaaaataatatataatatatttttattattcttatggtgcaaataaagcacattgtgcattggaaataaaaacggcaaattaattacctttgtttttaaaggtttattaaattggcggttttttgtttgaaaaaatgcttgtgatacatcattttttgatgtatcatacacacatcggcagacaacaagcctgccgccgccaccttgacggtttcaaggataaacgaaaggatttcaaa

SEQ ID NO: 45 - DNA, 356 bp, Neisseria meningitides
gataatatccccccgtccgcaaccgttcaaacgactaaggaggcaaaatggcatatcggttcaaaacaggcgtgattgccggaatcccgactgtatctactgaaaaataatatattgaaaaataatatataatatatttttattattcttatggtgcaaataaagcacattgtgcattggaaataaaaacggcaaattaattacctttggtttcaaaggtttattaaatttgccgttttttgtttgaaaaagtgcttgtgatacatcattttttgatgtagtatacacacatcggcagacaacaagcctgccaccgacaccttgacggcttcaaggataaacgaaaggatttcaaa

SEQ ID NO: 46 - DNA, 355 bp, Neisseria meningitides
aattgaatccgcaatggtgaaatatcgacaatgaacgacaatacacacacccttcccccgcgccacctgtccgtcgctcccatgctcgactgtatctactgaaaaataatatattgaaaaataatatataatatatttttattattcttatggtgcaaataaagcacattgtgcattggaaataaaaacggcaaattaattacctttgtttttaaaggtttattaaattggcggttttttgtttgaaaaaatgcttgtgatacatcattttttgatgtagtatacacacatcggcagacaacaagcctgccaccgacaccttgacggcttcaaggataaacgaaaggatttcaaa

SEQ ID NO: 47 - DNA, 356 bp, Neisseria meningitides
aattgaatccgcaatgatgaaatatcgacaatgaacgacaatacacacacccttcccccgcgccgcctttccgtcgccccgatgctcgactgtatctactgaaaaataatatattgaaaaataatatataatatatttttattattcttatggtgcaaataaagcacattgtgcattggaaataaaaacggcaaattaattacctttgttttcaaagatttattaaatttgccgttttttgtttaaaaaagtgcttgtgatacatcatttaatgatgtaatatacacacatggacagacaacaagcctgccaccgacaccttgacggattcaaggataaacgaaaggatttcaaaa

SEQ ID NO: 48 - DNA, 186 bp, Neisseria meningitides
atctaaccctatcagcaaacggcaaattaattacctttggtttcaaaggtttattaaatttgccgttttttgtttaaaaaagtgcttgtgatacatcatttaatgatgtaatatacacacatggacagacaacaagcctgccaccgacaccttgacggattcaaggataaacgaaaggatttcaaa

SEQ ID NO: 49 - DNA, 187 bp, Neisseria meningitides
atttaattctattaaataaacggcaaattaattacctttggtttcaaaggtttattaaatttgccgttttttgtttaaaaaagtgcttgtgatacatcatttaatgatgtaatatacacacatggacagacaacaagcctgccaccgacaccttgacggattcaaggataaacgaaaggatttcaaa

SEQ ID NO: 50 - protein, 149 aa, Listeria monocytogenes
MTIKLLDEFLKKHDLTRYQLSKLTGISQNTLKDQNEKPLNKYTVSILRSLSLISGLSVSDVLFELEDIEKNSDDLAGFKHLLDKYKLSFPAQEFELYCLIKEFESANIEVLPFTFNRFENEEHVNIKKDVCKALENAITVLKEKKNELL

SEQ ID NO: 51 - protein, 159 aa, Listeria monocytogenes
MSIKLLDEFLKKHNKTRYQLSKLTGISQNTLNDYNKKELNKYSVSFLRALSMCAGISTFDVFIELAELEKSYDDLAGFKYLLDKHKLSFPTQEFELYCLIKEFESANIEVLPFTFNRFENETHADIEKDVKKALNNAIAVLEEKKRRTVIKTIDYYDYS

SEQ ID NO: 52 - protein, 153 aa, Listeria monocytogenes
MNILDEFLNEHQITRYRLSKITGISNQLLLQYTKKTLEEYPVWLLRALAAATDQTIEEVLNKLEILETEKHQLYGIRSFLEKYNCSFPQEEWMLYRALYLVEALNMDLEEMKFDRFEKEEHANIEKDVQEAVSNAVSTIDMIRRKKLKGHFKN

SEQ ID NO: 53 - protein, 149 aa, Listeria monocytogenes
MKTNLLDTFLKRHGITRYRLSKLAGISQNTLKDYTEKSLNKYTVSFLRSLSFVTGEDVTDVLLELAEIENGYDDLAGFKYLLDKYKLSFPALEFELYCIIKEFESANIEISPFTFNRFENETHVDIEKDVKKALQNAVTVLEERKEELL

SEQ ID NO: 54 - protein, 149 aa, Listeria seeligeri
MKINLLDEFLKRHNITRYRLSKLAGISQNTLKDYTEKSLNKYTVSFLRSLSFATGESVTDILLELAELEKDYDDLAGFKYLLDKYKLAFPALEFELYCLIKEFESANIEISPFTFNRFESETHTDIEKDVKKALQNAVTVLEERKEELL

SEQ ID NO: 55 - protein, 174 aa, Enterococcus rivorum
MNKFIIHYLKIERKQTMNLLDKFLNKRNLTRQQLSNISGYSTGRLFDYNNKELNKYPVALLRTLAKISSMSLTDTLKELEEIEASYDSLLGFRKLLEQYELSFPDLEFELYCTIKDLESLKVKVEPFTFNRFEEEGHNNIASDCRKAMENAISMLSEALENVRKGKAPFEDEEI

SEQ ID NO: 56 - protein, 155 aa, Leuconostoc gelidum
MKLDDYLKLNNTTRYEVAKISGIPETSFKSIRNRDVNNLSGRFYRAIGLVLGKTGGQVYDEITADENTVFNFLGKHHIHDKERVTELLDYMLYFKKHDIDVTNVSFNRFENEIENGHILGDEDDVLQVIDNLIESFKTMKENVEAGNLPTPEKMD

SEQ ID NO: 57 - protein, 165 aa, Listeria monocytogenes
MNNHVIDLTNKKFGRLTVKEFVRSENGNALWNCFCVCGNEKEVLAQHLKRGHVQSCGCLARDNGRKHADKNLRSETAQKNALKRKLEVDAVDGTMKSALTRSLSARNKSGIKGVRWDEKRNKWEASITFQKKLHFLGRFEKKDDAVKARRDAEDKYFKPILDKMN

SEQ ID NO: 58 - protein, 150 aa, Listeria monocytogenes
MKGFLKRYAQEKKGWSLYKLAKESGIQDTTLSFANSKSVHNLSALNIKLISEAVGETPGTVLDELTELEKEMEMETTYWYNEGTGTLLTWKEYKAKIESEARDWLEDLQEEEEELDDSDKTSLETLVQLSFENESDFVLSDSEGNPIKEW

SEQ ID NO: 59 - protein, 148 aa, Unknown
  Description of Unknown Listeria phage sequence
MNELRSLEMSINAKDYATRLESGEGSLYIRFGDSEDYPVHASTNSTIKETFIELFKNGWNGYEEDEQELAEDMQEIAQELILEELTDIFEEYEFSTDEIDTDLFSGFTFHVDMDNDEAVYLMDAINATKYFEARPSSWYALLEVSYCG

SEQ ID NO: 60 - protein, 173 aa, Enterococcus faecalis
MKGLLELSTIDLFLKKYGITRNKVATQNIKHKISNNALAQANLRPVETYSVKLILGLSEAVNEAPEKVMAQLLEIEKSQTNSESAQKKEAYQFGNIILEGILNTNRSTHEIRLVQYLGKRTLFCTYVSGVGAMNWSVSDYKEIAETLKIDDVDIRFRTSENDQFWDVSESYRY

SEQ ID NO: 61 - DNA, 89 bp, Artificial Sequence
  Description of Artificial Sequence Synthetic oligonucleotide
ggttcactgccgtataggcagctaagaaaagttcctttcccttcagtccagcctgtgccaggttcactgccgtgtaggcagctaagaaa

SEQ ID NO: 62 - protein, 79 aa, Neisseria meningitides; identical to SEQ ID NO: 11

SEQ ID NO: 63 - DNA, 30 bp, Artificial Sequence
  Description of Artificial Sequence Synthetic oligonucleotide
gggcccgtcgactggccactttcggacaag

SEQ ID NO: 64 - DNA, 32 bp, Artificial Sequence
  Description of Artificial Sequence Synthetic oligonucleotide
cccggggtcgactcacgcagatggcgggtcgt

SEQ ID NO: 65 - DNA, 20 bp, Artificial Sequence
  Description of Artificial Sequence Synthetic oligonucleotide
tggttcagccctcaacaact

SEQ ID NO: 66 - DNA, 18 bp, Artificial Sequence
  Description of Artificial Sequence Synthetic oligonucleotide
tcttgagcatggcgagca

SEQ ID NO: 67 - DNA, 20 bp, Artificial Sequence
  Description of Artificial Sequence Synthetic oligonucleotide
gcctcggttcaacagtacga

SEQ ID NO: 68 - DNA, 21 bp, Artificial Sequence
  Description of Artificial Sequence Synthetic oligonucleotide
aacgtggtactccatcgcttt

SEQ ID NO: 69 - DNA, 20 bp, Artificial Sequence
  Description of Artificial Sequence Synthetic oligonucleotide
agttcgcctttatggacgag

SEQ ID NO: 70 - DNA, 20 bp, Artificial Sequence
  Description of Artificial Sequence Synthetic oligonucleotide
atttcggctcaaggctgtta

SEQ ID NO: 71 - DNA, 20 bp, Artificial Sequence
  Description of Artificial Sequence Synthetic oligonucleotide
cgggtccaacttggtctatg

SEQ ID NO: 72 - DNA, 19 bp, Artificial Sequence
  Description of Artificial Sequence Synthetic oligonucleotide
tttcgtcgaacggcagata

SEQ ID NO: 73 - DNA, 21 bp, Artificial Sequence
  Description of Artificial Sequence Synthetic oligonucleotide
atgaagttcatcaaatacctc

SEQ ID NO: 74 - DNA, 20 bp, Artificial Sequence
  Description of Artificial Sequence Synthetic oligonucleotide
tcaggggttttcacgccggg

SEQ ID NO: 75 - DNA, 20 bp, Artificial Sequence
  Description of Artificial Sequence Synthetic oligonucleotide
aatacctcagcaccgctcac

SEQ ID NO: 76 - DNA, 20 bp, Artificial Sequence
  Description of Artificial Sequence Synthetic oligonucleotide
ttgccgtttacgacgttctc

SEQ ID NO: 77 - DNA, 20 bp, Artificial Sequence
  Description of Artificial Sequence Synthetic oligonucleotide
atgagatttcccggcgtgaa

SEQ ID NO: 78 - DNA, 20 bp, Artificial Sequence
  Description of Artificial Sequence Synthetic oligonucleotide
tcacgcagatggcgggtcgt

SEQ ID NO: 79 - DNA, 18 bp, Artificial Sequence
  Description of Artificial Sequence Synthetic oligonucleotide
tcaagaaagccggcatca

SEQ ID NO: 80 - DNA, 20 bp, Artificial Sequence
  Description of Artificial Sequence Synthetic oligonucleotide
tccttgatgtcctcgctcag

SEQ ID NO: 81 - DNA, 21 bp, Artificial Sequence
  Description of Artificial Sequence Synthetic oligonucleotide
gaaaagaaccgcctactcgtt

SEQ ID NO: 82 - DNA, 20 bp, Artificial Sequence
  Description of Artificial Sequence Synthetic oligonucleotide
tggctttcaggagttcatcc

SEQ ID NO: 83 - DNA, 20 bp, Artificial Sequence
  Description of Artificial Sequence Synthetic oligonucleotide
atgagcaccagaccctcaag

SEQ ID NO: 84 - DNA, 19 bp, Artificial Sequence
  Description of Artificial Sequence Synthetic oligonucleotide
gggctgtgtattcgacacg

SEQ ID NO: 85 - DNA, 22 bp, Artificial Sequence
  Description of Artificial Sequence Synthetic oligonucleotide
ctgcaagagtttctggatgatg

SEQ ID NO: 86 - DNA, 22 bp, Artificial Sequence
  Description of Artificial Sequence Synthetic oligonucleotide
ctttatctgcgacgagtgtgtc

SEQ ID NO: 87 - DNA, 22 bp, Artificial Sequence
  Description of Artificial Sequence Synthetic oligonucleotide
cgcttgtagtggttgtataccg

SEQ ID NO: 88 - DNA, 22 bp, Artificial Sequence
  Description of Artificial Sequence Synthetic oligonucleotide
aaagtagtgggcacaaacttcc

SEQ ID NO: 89 - DNA, 20 bp, Artificial Sequence
  Description of Artificial Sequence Synthetic oligonucleotide
gagatgcggttgagcttgtt

SEQ ID NO: 90 - DNA, 20 bp, Artificial Sequence
  Description of Artificial Sequence Synthetic oligonucleotide
gtcgacagcgtcctgaagag

SEQ ID NO: 91 - DNA, 20 bp, Artificial Sequence
  Description of Artificial Sequence Synthetic oligonucleotide
gggcgaagaaggaaatggtc

SEQ ID NO: 92 - DNA, 20 bp, Artificial Sequence
  Description of Artificial Sequence Synthetic oligonucleotide
caggtggcgtaggtagagaa

SEQ ID NO: 93 - DNA, 32 bp, Artificial Sequence
  Description of Artificial Sequence Synthetic oligonucleotide
cccgggccccatggtggccactttcggacaag

SEQ ID NO: 94 - DNA, 34 bp, Artificial Sequence
  Description of Artificial Sequence Synthetic oligonucleotide
cccgggaagcttggtttgaatccttgttggcgcc

SEQ ID NO: 95 - DNA, 35 bp, Artificial Sequence
  Description of Artificial Sequence Synthetic oligonucleotide
agccgaaatcggtagaacggcgaggcgccaacaag

SEQ ID NO: 96 - DNA, 19 bp, Artificial Sequence
  Description of Artificial Sequence Synthetic oligonucleotide
ctaccgatttcggctcaag

SEQ ID NO: 97 - DNA, 31 bp, Artificial Sequence
  Description of Artificial Sequence Synthetic oligonucleotide
cccgggccatggccagatttcccggcgtgaa

SEQ ID NO: 98 - DNA, 32 bp, Artificial Sequence
  Description of Artificial Sequence Synthetic oligonucleotide
cccgggaagctttcacgcagatggcgggtcgt

SEQ ID NO: 99 - DNA, 54 bp, Artificial Sequence
  Description of Artificial Sequence Synthetic oligonucleotide
acaagcggcacactgtgcctattgcgaattaggcacaatgtgcctaatctaacg

SEQ ID NO: 100 - DNA, 54 bp, Artificial Sequence
  Description of Artificial Sequence Synthetic oligonucleotide
ggttagattaggcacattgtgcctaattcgcaataggcacagtgtgccgcttgt

SEQ ID NO: 101 - DNA, 54 bp, Artificial Sequence
  Description of Artificial Sequence Synthetic oligonucleotide
acaagcgtcgtactgtgcctattgcgaattaggcacaatgtgcctaatctaacg

SEQ ID NO: 102 - DNA, 54 bp, Artificial Sequence
  Description of Artificial Sequence Synthetic oligonucleotide
cgttagattaggcacattgtgcctaattcgcaataggcacagtacgacgcttgt

SEQ ID NO: 103 - DNA, 54 bp, Artificial Sequence
  Description of Artificial Sequence Synthetic oligonucleotide
acaagcggcacactgtgcctattgcgagctagtcccaatgtgcctaatctaacg

SEQ ID NO: 104 - DNA, 54 bp, Artificial Sequence
  Description of Artificial Sequence Synthetic oligonucleotide
cgttagattaggcacattgggactagctcgcaataggcacagtgtgccgcttgt

SEQ ID NO: 105 - DNA, 54 bp, Artificial Sequence
  Description of Artificial Sequence Synthetic oligonucleotide
acaagcgtcgtactgtgcctattgcgagctagtcccaatgtgcctaatctaacg

SEQ ID NO: 106 - DNA, 54 bp, Artificial Sequence
  Description of Artificial Sequence Synthetic oligonucleotide
cgttagattaggcacattgggactagctcgcaataggcacagtacgacgcttgt

SEQ ID NO: 107 - DNA, 42 bp, Artificial Sequence
  Description of Artificial Sequence Synthetic oligonucleotide
ggccactttcggacaagcgtcgtactgtgcctattgcgaatt

SEQ ID NO: 108 - DNA, 42 bp, Artificial Sequence
  Description of Artificial Sequence Synthetic oligonucleotide
aattcgcaataggcacagtacgacgcttgtccgaaagtggcc

SEQ ID NO: 109 - DNA, 51 bp, Artificial Sequence
  Description of Artificial Sequence Synthetic oligonucleotide
tgacgttagattaggcacattgggactgcttcgcaataggcacagtgtgcc

SEQ ID NO: 110 - DNA, 51 bp, Artificial Sequence
  Description of Artificial Sequence Synthetic oligonucleotide
ggcacactgtgcctattgcgaagcagtcccaatgtgcctaatctaacgtca

SEQ ID NO: 111 - DNA, 26 bp, Artificial Sequence
  Description of Artificial Sequence Synthetic oligonucleotide
tcggctgcgcgcgcctggctgatgcc

SEQ ID NO: 112 - DNA, 26 bp, Artificial Sequence
  Description of Artificial Sequence Synthetic oligonucleotide
ggcatcagccaggcgcgcgcagccga

SEQ ID NO: 113 - DNA, 28 bp, Artificial Sequence
  Description of Artificial Sequence Synthetic oligonucleotide
cagctcggctgcggcccgctggctgatg

SEQ ID NO: 114 - DNA, 28 bp, Artificial Sequence
  Description of Artificial Sequence Synthetic oligonucleotide
catcagccagcgggccgcagccgagctg

SEQ ID NO: 115 - DNA, 31 bp, Artificial Sequence
  Description of Artificial Sequence Synthetic oligonucleotide
cagctcggctgcggccgcctggctgatgccg

SEQ ID NO: 116 - DNA, 31 bp, Artificial Sequence
  Description of Artificial Sequence Synthetic oligonucleotide
cggcatcagccaggcggccgcagccgagctg

SEQ ID NO: 117 - DNA, 34 bp, Artificial Sequence
  Description of Artificial Sequence Synthetic oligonucleotide
gtaatagcgcatcaccgcgtcactgaggccgagc

SEQ ID NO: 118 - DNA, 34 bp, Artificial Sequence
  Description of Artificial Sequence Synthetic oligonucleotide
gctcggcctcagtgacgcggtgatgcgctattac

SEQ ID NO: 119 - DNA, 45 bp, Artificial Sequence
  Description of Artificial Sequence Synthetic oligonucleotide
tagttgcggccgcaaaatggatgaatggtcaagaattaaaaaaag

SEQ ID NO: 120 - DNA, 40 bp, Artificial Sequence
  Description of Artificial Sequence Synthetic oligonucleotide
gcggccgcaggcaaaggatattagattaaatccgcgtgac

SEQ ID NO: 121 - DNA, 39 bp, Artificial Sequence
  Description of Artificial Sequence Synthetic oligonucleotide
tagttgcggccgcaaaatggatgaagaaatttgaagccc

SEQ ID NO: 122 - DNA, 47 bp, Artificial Sequence
  Description of Artificial Sequence Synthetic oligonucleotide
gcggccgcaggcaaaggatattattttaatgaatccaaaagtttttg

SEQ ID NO: 123 - DNA, 18 bp, Artificial Sequence
  Description of Artificial Sequence Synthetic oligonucleotide
tatcctttgcctgcggcc

SEQ ID NO: 124 - DNA, 18 bp, Artificial Sequence
  Description of Artificial Sequence Synthetic oligonucleotide
ccattttgcggccgcaac

SEQ ID NO: 125 - DNA, 28 bp, Artificial Sequence
  Description of Artificial Sequence Synthetic oligonucleotide
cccgggccatggagcctcacctccggcg

SEQ ID NO: 126 - DNA, 38 bp, Artificial Sequence
  Description of Artificial Sequence Synthetic oligonucleotide
cccgggaagcttctcgaaccgatgaataaattatatgt

SEQ ID NO: 127 - DNA, 34 bp, Artificial Sequence
  Description of Artificial Sequence Synthetic oligonucleotide
cccgggccatggaattgaatccgcaatggtgaaa

SEQ ID NO: 128 - DNA, 37 bp, Artificial Sequence
  Description of Artificial Sequence Synthetic oligonucleotide
cccgggaagctttttgaaatcctttcgtttatccttg

SEQ ID NO: 129 - DNA, 780 bp, Pectobacterium phage ZF40
agcctcacctccggcgttgccgtggcgctgtgtgatttacaggaaataaaaaggccacgaatgcggccttagcgattaaaaaatatgaaatgccttgcttgttcgcgattgcgaacatataatttattcatcggttcgagatggctcgaatcgctcctaacgaggattccacaatgtctactgcttacatcatctttaactcatccgtcgcggccgtagttgatactgagatcgctaatggcgctaatgtcacattctcaacagtgaccgttaaagaagaaattaacgcgaaccgtgatttcaatctggttaacgctcagaacgggaaaatctcacgcgcaaaacgctggggaaacgaggcgtcaaaatgtgagtattttggccgagaaataaacccaaccgagtttttcatcaaataatgtggtcaaaatgacaaacaaagaacttcaggcaatcagaaaactgttaatgctggatgtatcagaagcggctgaacacattggccgcgtttccgcccggagttggcaatattgggagtctggacgctctgctgttcctgatgatgttgagcaggaaatgttggatttagcgtcagtcaggatagaaatgatgtccgctatagacaagcgtctcgccgatggcgaacgtcctaaattacgtttttataacaagttggatgaatacctggctgacaaccccgatcacaatgtgatcgggtggcgtctgagccagtctgttgccgcactctattacactgagggtcacgcggatttaatctaa

SEQ ID NO: 130 - DNA, 697 bp, Neisseria meningitidis
tcccaattacctgtttgaagcagtatttgtttctcaaatgaccaatttttaaccaaaggccgctaatgtggccgttttttttgttctcatactcttctaatttagggtctctgcctccaagctcccggtctcgccgccgacggctcgggagcagggcatagccataaaagcttacattgtgtgctagactatatcaaactacaactacgaaaggaaatccgaacactatgaataaaacttataaaattggaaaaaatgccgggtatgatggctgcggtctttgtcttgcggccatttctgaaaatgaagctatcaaagttaagtatttgcgcgacatttgtcctgattacgatggcgatgataaagctgaggattggctgagatggggaacggacagccgcgtcaaagcagccgctcttgaaatggagcagtacgcatatacgtcggttggtatggcctcatgttgggagtttgttgaactatgaagaaatttgaagcccctgaaattggctatacacctgccaatcttaaagcactgagaaaacaatttgggcttacacaagctcaggtagcagaaattactggtacaaaaaccggatacagcgtccgcaggtgggaagcagcaattgatgccaaaaatcgcgcggatatgccgctcgtaaaatggcaaaaacttttggattcattaaaataatga

SEQ ID NO: 131 - DNA, 138 bp, Unknown
  Description of Unknown promoter sequence
gggcgttaggggaaatgaattcggacaagcggcacattgtgcctattgcgtattaggcacaatgtgcctaatctagcgtcatgccagccacaacggcgaggcgaacccaaggagagacaccatgactaagaccgcaca

SEQ ID NO: 132 - DNA, 138 bp, Unknown
  Description of Unknown promoter sequence
gggcgttaggggaaatgaattcggacaagcggcacattgtgcctattgcggattaggcacaatgtgcctaatctagcgtcatgccagccacaacggcgaggcgaacccaaggagagacaccatgactaagaccgcaca

SEQ ID NO: 133 - DNA, 145 bp, Unknown
  Description of Unknown promoter sequence
tggcgctagggggaatgaattcggacaagcggcacattgtgcctattgcgtattaggcacaatgtgcctaatctagcctcatgccagccacaacggcgaggcgctaacaaggatcgaagctatgaaaatcaccaatgacaccacc

SEQ ID NO: 134 - DNA, 143 bp, Unknown
  Description of Unknown promoter sequence
aatcggtagtggccactttcggacaagcggcacactgtgcctattgcgaattaggcacaatgtgcctaatctaacgtcatgccagccacaacggcgaggcgccaacaaggattcaaaccatgaagttcatcaaatacctcagc

SEQ ID NO: 135 - DNA, 143 bp, Unknown
  Description of Unknown promoter sequence
aatcggtagtggccactttcggacaagcggcacattgtgcctattgcgaattaggcacaatgtgcctaatctaacgtcatgccagccacaacggcgaggcgccaagaaggatagaagccatgaaaatcaccaacgacaccacc

SEQ ID NO: 136 - DNA, 159 bp, Unknown
  Description of Unknown promoter sequence
aatcggtagtgggccactttcggacaagcggcacattgtgcctattgcgtattaggcacaatgtgcctaatctaacgtcatgccaaccacaacggcgaggcgcaaacaaggatagacaccatgagcagcacgatttcagaccgcatcatctcccgctcg

SEQ ID NO: 137 - DNA, 156 bp, Unknown
  Description of Unknown promoter sequence
ggtcgctagagggaattcattcggataagcggcacattgtgcctattgtgcattaggcacaatgtgcctaatatggcgtcatgccagccacaatggcgaggcgccacgaaggaacgatgccatggaaaagaagctttcagatgctcaggttgcgct

SEQ ID NO: 138 - DNA, 54 bp, Artificial Sequence; identical to SEQ ID NO: 99
  Description of Artificial Sequence Synthetic oligonucleotide

SEQ ID NO: 139 - DNA, 110 bp, Unknown
  Description of Unknown parent phage sequence
tggccactttcggacaagcggcacactgtgcctattgcgaattaggcacaatgtgcctaatctaacgtcatgccagccacaacggcgaggcgccaacaaggattcaaacc

SEQ ID NO: 140 - DNA, 85 bp, Unknown
  Description of Unknown suppressor phage sequence
tggccactttcggacaagcggcacactgtgcctaatctaacgtcatgccagccacaacggcgaggcgccaacaaggattcaaacc

SEQ ID NO: 141 - protein, 79 aa, Unknown; identical to SEQ ID NO: 1
  Description of Unknown phage sequence

SEQ ID NO: 142 - protein, 73 aa, Unknown
  Description of Unknown phage sequence
MKPDASNHNPDPRYLRGLLKKAGISQRRAAELLGLSDRVMRYYLSEDIKEGYRPAPYTVQFALECLANDPPSA

SEQ ID NO: 143 - protein, 73 aa, Unknown
  Description of Unknown phage sequence
MKPDASSHNPDPRYLRGLLKKAGISQRRAAELLGLSDRVMRYYLSEDVKEGYRPAPYTVQFALECLANDPPSA

SEQ ID NO: 144 - protein, 71 aa, Pseudomonas sp.
MKPDASSHNPDPRYLRGLVERSSLSQRRIAELLGITDRAMRYYLSDEVSATFRPAPYPVQFALECLASSES

SEQ ID NO: 145 - protein, 78 aa, Pseudomonas fluorescens
MKPDASNHNPSPHYLRGVLDQAGISQREAATMLGLSDRVIRYYLSDTTSPSHRPAPYVVQFAIECLADHPAQHKRDQP

SEQ ID NO: 146 - protein, 77 aa, Acinetobacter brisouii
MIPDSNNYNPDPSYLRYLVDKANVSQRKAAHIIGITERTMRYYMSDTASETYRKAPYAVQFALESLAGEWLGKEFKP

SEQ ID NO: 147 - protein, 82 aa, Unknown
  Description of Unknown phage sequence
MQLKPRNTVPRPDASSHNPDPRYLRGLLKKAGISQRRAAELLGLSDRVMRYYLSEDIKEGYRPAPYTVQFALECLANDPPSA

SEQ ID NO: 148 - protein, 72 aa, Pseudomonas aeruginosa
MKPDSANHNPDPRYLRGLVDRSGISQRQAAELLGLSWPGFRNYLRDESHQLYRAAPYTVQFALECLAESPSS

SEQ ID NO: 149 - protein, 116 aa, Pectobacterium phage ZF40
MTNKELQAIRKLLMLDVSEAAEHIGRVSARSWQYWESGRSAVPDDVEQEMLDLASVRIEMMSAIDKRLADGERPKLRFYNKLDEYLADNPDHNVIGWRLSQSVAALYYTEGHADLI

SEQ ID NO: 150 - protein, 119 aa, Vibrio parahaemolyticus
MTNQELKQLRRLLFIEVSEAAALIGECEPRTWQRWEKGDRAIPNDVSREIQMLALTRLERLQVEFDETDPNYRYFETFDEYKAYGGTGNELKWRLAQSVATSLLCETEADKWREEETID

SEQ ID NO: 151 - protein, 119 aa, Vibrio cyclitrophicus
MTNHELKQLRRLLFLEVSEAAELIGECEPRTWQRWEKGDRAIPNDVSRVIQMLALTRLERVELLHDVGDPNYRYFDSFEEYKASGGAGSELKWRLAQSVATALLCESEADKWREEETID

SEQ ID NO: 152 - protein, 121 aa, Phaseolibacter flectens
MTNFELKQLRKLFFLTTAEAAEHIGGVEPRAWQRWEKGERAIPADVSEHMQMLALTRYERLQREPEISNVAYQYCDSLDSFVAAGGVRNVTMWRLAQSVASELTLEKFAFNQRDSETIGFD

SEQ ID NO: 153 - protein, 117 aa, Serratia marcescens
MTNKELKALRQILALECSEAAEHIGGVSTRSWQRWEDGTRPVPDDAANLITDYAAIRDRLVEERYKLFKKTGDVITLQFYMSVDEFEAATGKRDVVMWKITNSVAGECLSAGIATLI

SEQ ID NO: 154 - protein, 123 aa, Proteus penneri
MTNLELRQLRQLLFLSPSEASSEIGRVETRTWQRWEKGDRAIPYDVIQQMQMLSLTRLELLSVEADHSHYIYQYFDSLDEYVKAGGTHSVIKWRLSQSISAQLLSEKMAEIWQDEEIIKNDNA

SEQ ID NO: 155 - protein, 120 aa, Shewanella xiamenensis; identical to SEQ ID NO: 8

SEQ ID NO: 156 - protein, 125 aa, Oceanimonas smirnovii; identical to SEQ ID NO: 10

SEQ ID NO: 157 - protein, 151 aa, Brackiella oedipodis; identical to SEQ ID NO: 9

SEQ ID NO: 158 - DNA, 140 bp, Pectobacterium phage ZF40; identical to SEQ ID NO: 40

SEQ ID NO: 159 - DNA, 279 bp, Vibrio parahaemolyticus
agcatccaccttccccagtcatgttcacatgataagatgaagaaaaaataggcgccctcaacttgacggcgcctatgatggacagctcaacgtattttgacttttggtggcagtattgagcgattgacgttgttagtttttaaggatattcggaaaagagtgtgtatcgggtaagttaaaataatatcaaattaacacttgcaaccggtcgcaattgcgaccataatgtattcaacggttcggaactggttcgaatcgactcaagaaagtgagatacat

SEQ ID NO: 160 - DNA, 214 bp, Vibrio cyclitrophicus
tcttgaaacctgttacataattcatagttttgattagtgtaacggtaatcaaaactcgtcacaagatatacaaaacgggtattcggaaaaagagtgtgtatcggataagttaaaataaattcaaattaacacttgcaaatggtcgcaattgcgaccataatatcttcaacggttcggaactggttcgaatcgactcaagaaagtgagatacatc

SEQ ID NO: 161 - DNA, 274 bp, Phaseolibacter flectens; identical to SEQ ID NO: 35

SEQ ID NO: 162 - DNA, 164 bp, Serratia marcescens
tcaacatttctccaagcgtaaaaaagccctgcagagcagggctagattaatgattaagtgagcatcacgcagcagcctcggaaagctgctctgtgatgaaatttgtgactagcatcactttacatggtcgcgatcgcgacctataatgttttcaacggttcggg

SEQ ID NO: 163 - DNA, 272 bp, Proteus penneri
tgcgcatatacaccccctacggagtgtccgagtttagttaaaaggatgcagacacacagctcttgtgtgaagtgattagtgtgtgattgatactgtggtctgcatatacgaaaaaagaccgcctaagcgatcttctgaatgtgattcaagtcaaaattttaagttatgtatattaatttcataatatcgcttgcctttggtcgctattgcgaccatgctgtattcatcgggtaggcaataaggcagacccaactcaactaagtgagaatatt

SEQ ID NO: 164 - DNA, 91 bp, Shewanella xiamenensis
ataaaacacttgcaatcggtcgcaatcgcgaccataatatatttaacggttcgggagtggctcgaatcgactcaataaagtgagaaatatc

SEQ ID NO: 165 - DNA, 156 bp, Oceanimonas smirnovii
aaagctcgcccccacgcttcaccgttaacccccaggaagcagattggctgatttttgtattttctttactttatctcttgcactgttcgcaattgcgaactaagatggaaccagattcgagattggctcgaatcacctctaaagagagacacatca

SEQ ID NO: 166 - DNA, 295 bp, Brackiella oedipodis
tgaaagcgcagagccgattaatttccgcttcgacctaggtgctgacaacacctcgccgaaatacgcttgctgtgatggcgaaaaatcacgtggcgaaatggctgggcctggtatccagggaacaacgagcgaaacgtcattaaacaatgcggacaatcgggagcagaccgacattactgtcgcttaccttcgtaggcgactttcaaaagcaactcgttcgggttgtttttgaaagataattttttaaccgcaattgagaaccgctcagttgcattttttcttcaggagataaccc

SEQ ID NO: 167 - protein, 77 aa, Neisseria meningitides
MRRLWRAGMIDNPELGYTPANLKAIRQKYGLTQKQVANIAGATLSTAQKWEAAMSLKTHSDMPHTRWLLLLEYVRNL

SEQ ID NO: 168 - protein, 77 aa, Neisseria meningitides
MRRLWRAGMIDNPELGYTPANLKAVRQKYGLTQKQVANIAGATLSTAQKWEAAMSLKTHSDMPHTRWLLLLEYVRNL

SEQ ID NO: 169 - protein, 79 aa, Neisseria meningitides
MKMRRIWRTWMIDNPELGYTPANLKAVRQKYGLTQKQVADITGATLSTAQKWEAAMSLKTHSDMPHTRWLLLLEYVRNL

SEQ ID NO: 170 - protein, 79 aa, Neisseria meningitides; identical to SEQ ID NO: 11

SEQ ID NO: 171 - protein, 79 aa, Neisseria meningitides
MKMRRIWGILMIDSPELGYTPANLKAIRQKYGLTQKQVANIAGATLSTAQKWEAAMSLKTHSDMPHTRWLLLLEYVRNL

SEQ ID NO: 172 - protein, 79 aa, Neisseria meningitides
MKMRRIWRTWMIDSPELGYTPANLKAVRQKYELTQQQVADITGATLSTAQKWEAAMSLKTHSDMPHTRWLILLEYVRKL

SEQ ID NO: 173 - protein, 77 aa, Neisseria meningitides
MRRIWGILMIDRPELGYTPANLKAVRQKYGLTQNQVADITGTTLSTAQKWEAAMSLKTHSDMPHTRWLILLEYVRKL

SEQ ID NO: 174 - DNA, 356 bp, Neisseria meningitides; identical to SEQ ID NO: 43

SEQ ID NO: 175 - DNA, 355 bp, Neisseria meningitides; identical to SEQ ID NO: 44

SEQ ID NO: 176 - DNA, 356 bp, Neisseria meningitides; identical to SEQ ID NO: 45

SEQ ID NO: 177 - DNA, 355 bp, Neisseria meningitides; identical to SEQ ID NO: 46

SEQ ID NO: 178 - DNA, 356 bp, Neisseria meningitides; identical to SEQ ID NO: 47

SEQ ID NO: 179 - DNA, 186 bp, Neisseria meningitides; identical to SEQ ID NO: 48

SEQ ID NO: 180 - DNA, 187 bp, Neisseria meningitides; identical to SEQ ID NO: 49

SEQ ID NO: 181 - DNA, 47 bp, Unknown
  Description of Unknown phage sequence
ttaatttataaaaacacttgactactacgattattcgtagtatagtg

SEQ ID NO: 182 - DNA, 47 bp, Unknown
  Description of Unknown phage sequence
taaaattgataaaactattgactactacgtatattcgtagtataatg

SEQ ID NO: 183 - DNA, 47 bp, Unknown; identical to SEQ ID NO: 182
  Description of Unknown phage sequence

SEQ ID NO: 184 - DNA, 47 bp, Unknown
  Description of Unknown phage sequence
taaatttaataaaactattgactactacggcgattcgtagtatacta

SEQ ID NO: 185 - DNA, 47 bp, Unknown; identical to SEQ ID NO: 184
  Description of Unknown phage sequence

SEQ ID NO: 186 - DNA, 20 bp, Unknown
  Description of Unknown WT palindrome sequence
tactacgtatattcgtagta

SEQ ID NO: 187 - DNA, 20 bp, Artificial Sequence
  Description of Artificial Sequence Synthetic oligonucleotide
aactacgtatattcgtagtt

SEQ ID NO: 188 - protein, 73 aa, Listeria monocytogenes
MTIKLLDEFLKKHDLTRYQLSKLTGISQNTLKDQNEKPLNKYTVSILRSLSLISGLSVSDVLFELEDIEKNSD

SEQ ID NO: 189 - protein, 73 aa, Listeria monocytogenes
MKTNLLDTFLKRHGITRYRLSKLAGISQNTLKDYTEKSLNKYTVSFLRSLSFVTGEDVTDVLLELAEIENGYD

SEQ ID NO: 190 - protein, 73 aa, Listeria seeligeri
MKINLLDEFLKRHNITRYRLSKLAGISQNTLKDYTEKSLNKYTVSFLRSLSFATGESVTDILLELAELEKDYD

SEQ ID NO: 191 - protein, 73 aa, Listeria monocytogenes
MSIKLLDEFLKKHNKTRYQLSKLTGISQNTLNDYNKKELNKYSVSFLRALSMCAGISTFDVFIELAELEKSYD

SEQ ID NO: 192 - protein, 87 aa, Enterococcus rivorum
MNKFIIHYLKIERKQTMNLLDKFLNKRNLTRQQLSNISGYSTGRLFDYNNKELNKYPVALLRTLAKISSMSLTDTLKELEEIEASYD

SEQ ID NO: 193 - protein, 71 aa, Listeria monocytogenes
MNILDEFLNEHQITRYRLSKITGISNQLLLQYTKKTLEEYPVWLLRALAAATDQTIEEVLNKLEILETEKH

SEQ ID NO: 194 - protein, 69 aa, Leuconostoc gelidum
MKLDDYLKLNNTTRYEVAKISGIPETSFKSIRNRDVNNLSGRFYRAIGLVLGKTGGQVYDEITADENTV

SEQ ID NO: 195 - protein, 72 aa, Listeria monocytogenes
MAGFIKKYLENKEWTIYQLGNATGLAHQTIRSADSKTVDQMSAKNVRLIADVFEFTPGEILDEFYEIEEEIN

SEQ ID NO: 196 - DNA, 33 bp, Unknown
  Description of Unknown promoter sequence
tattgactactacggcgattcgtagtatactat

SEQ ID NO: 197 - DNA, 33 bp, Unknown
  Description of Unknown promoter sequence
tactgactactacggcgattcgtagtatactat

SEQ ID NO: 198 - DNA, 33 bp, Unknown
  Description of Unknown promoter sequence
tatcgactactacggcgattcgtagtatactat

SEQ ID NO: 199 - DNA, 32 bp, Unknown
  Description of Unknown promoter sequence
tattactactacggcgattcgtagtatactat

SEQ ID NO: 200 - DNA, 33 bp, Unknown
  Description of Unknown promoter sequence
tattgactactacggcgattcgtagcatactat

SEQ ID NO: 201 - DNA, 33 bp, Unknown
  Description of Unknown promoter sequence
tattgactactacggcgattcgtagtataccat

SEQ ID NO: 202 - DNA, 33 bp, Unknown; identical to SEQ ID NO: 196
  Description of Unknown promoter sequence

SEQ ID NO: 203 - DNA, 37 bp, Unknown
  Description of Unknown phage sequence
ttgataaaactattgactactacgtatattcgtagta

SEQ ID NO: 204 - DNA, 37 bp, Unknown
  Description of Unknown phage sequence
ttataaaaacacttgactactacgattattcgtagta

SEQ ID NO: 205 - DNA, 37 bp, Unknown
  Description of Unknown phage sequence
ttaataaaactattgactactacggcgattcgtagta

SEQ ID NO: 206 - DNA, 37 bp, Unknown; identical to SEQ ID NO: 205
  Description of Unknown phage sequence

SEQ ID NO: 207 - DNA, 37 bp, Unknown
  Description of Unknown phage sequence
ttaataaaactatcgactactacgattattcgtagta

SEQ ID NO: 208 - DNA, 20 bp, Unknown
  Description of Unknown WT palindrome sequence
tactacgtatattcgtagta

SEQ ID NO: 209 - DNA, 20 bp, Artificial Sequence
  Description of Artificial Sequence Synthetic oligonucleotide
aactacgtatattcgtagtt

SEQ ID NO: 210 - DNA, 20 bp, Artificial Sequence
  Description of Artificial Sequence Synthetic oligonucleotide
aacaacctatattggttgtt

SEQ ID NO: 211 - protein, 6 aa, Unknown
  Description of Unknown WT sequence
DYYDYS

SEQ ID NO: 212 - DNA, 23 bp, Unknown
  Description of Unknown WT sequence
gactactacgattattcgtagta

SEQ ID NO: 213 - DNA, 23 bp, Artificial Sequence
  Description of Artificial Sequence Synthetic oligonucleotide
gactattatgactattcttaaag

SEQ ID NO: 214 - DNA, 298 bp, Unknown
  Description of Unknown Listeria phage sequence
ttacttcacctcttgacaacattatacgaacaaacgttcttaaaatcaagtgttaaaaagtgttgtattacataaaaatctatgtaataatattcacatgaacgattttcgttcattatttcattcaactattagctgtttgacatcccgttttacatctgaatataacagcaacctcgaattttttcggggtatttttttattttaaaataaaattgataaaactattgactactacgtatattcgtagtataatgtgaatatagtaaagaaaacgattgaaaaggatggatgacaa

SEQ ID NO: 215 - DNA, 305 bp, Unknown
  Description of Unknown Listeria phage sequence
agttcctcgttttctctcgttggaagaagaagaaacgagaaactaaaattataaataaaaagtaacctatttttctgtagattgctttttatcatttatatagaagaaagccgctttttattagattataattgatgttttttgatttatatttcacttcttgtgcaaataacgatatagtagcaacctcggactttttagttcggtgtatttttttgaaattaatttataaaaacacttgactactacgattattcgtagtatagtgtaagtatagtaaagataacgaaacggaggaatttaaa

SEQ ID NO: 216 - DNA, 299 bp, Unknown
  Description of Unknown Listeria phage sequence
ttacttcacctcttgacaacattatacgaacaaacgttcttaaaatcaagtgttaaaaagtgttgtattacataaaaatctatgtaataatattcacatgaacgattttcgttcattatttcattcaactattagctgtttgacatcccgttttacatctgaatataacagcaacctcgaatttttcggggtatttttttatattgaaaataaatttaataaaactattgactactacggcgattcgtagtatactatgtatatagtaaagaaaacaattgaaaaggatggatgacaaa

SEQ ID NO: 217 - DNA, 299 bp, Unknown; identical to SEQ ID NO: 216
  Description of Unknown Listeria phage sequence

SEQ ID NO: 218 - DNA, 353 bp, Unknown
  Description of Unknown Listeria phage sequence
agtataaaatattatctaatgttatctagtgttatttagttaaggtgttaataaggtgttttttcttctttctctattatatagaagaaaagtgttttatttttattgaacttcataaaaatctatgtaataatattcacgtgagcgagactatcgttcaatcaattatttcattcatctcatttcgctgtttgacatcccgtttttttcatctgaatataacagcaacctcgaataaattttcggggtatttttttattttaaaataaatttaataaaactatcgactactacgattattcgtagtataatgtaaatatagtaaacaaaccaactaaaaaggatgatgaaaa

SEQ ID NO: 219 - protein, 149 aa, Listeria monocytogenes; identical to SEQ ID NO: 50

SEQ ID NO: 220 - protein, 159 aa, Listeria monocytogenes; identical to SEQ ID NO: 51

SEQ ID NO: 221 - protein, 149 aa, Listeria monocytogenes; identical to SEQ ID NO: 53

SEQ ID NO: 222 - protein, 149 aa, Listeria monocytogenes
MKINLLDEFLKRHNITRYRLSKLAGISQNTLKDYNEKSLNKYTVSFLRSLSFVTGEDVTDVLLELAEIENGYDDLAGFKYLLDKYKLSFPALEFELYCIIKEFESANIEISPFTFNRFESETHTDIEKDVKKALQNAVTVLEERKEELL

SEQ ID NO: 223 - protein, 149 aa, Listeria seeligeri; identical to SEQ ID NO: 54

SEQ ID NO: 224 - protein, 174 aa, Enterococcus rivorum; identical to SEQ ID NO: 55

SEQ ID NO: 225 - protein, 153 aa, Listeria monocytogenes; identical to SEQ ID NO: 52

SEQ ID NO: 226 - protein, 155 aa, Leuconostoc gelidum; identical to SEQ ID NO: 56

SEQ ID NO: 227 - protein, 145 aa, Lactobacillus parabuchneri
MHTIDDFLKYSDLTRYDIAKISGISETTLADANQRPVNKMTVKVVQAIAMGVGMTPGRTLDELLKVEGNPIMQFIQAHPYMNHDLVNEVKEFMSDAAEKGIFVENLNFDQYYNQPDTNERAEIALRNKFLGLKDMVKQMEDSQSE

SEQ ID NO: 228 - protein, 173 aa, Enterococcus faecalis; identical to SEQ ID NO: 60

SEQ ID NO: 229 - protein, 142 aa, Listeria monocytogenes
MAGFIKKYLENKEWTIYQLGNATGLAHQTIRSADSKTVDQMSAKNVRLIADVFEFTPGEILDEFYEIEEEINNDAIIQELINVFEKYGYNTDEISLELLDGEKIKLEMSDDTITQLADAVNATKHFTAYVDASTDFMIIEKI

SEQ ID NO: 230 - protein, 116 aa, Pectobacterium phage ZF40; identical to SEQ ID NO: 149